(19) United States
(12) Patent Application Publication
(10) Pub. No.: US 2026/0176675 A1
(43) Pub. Date: Jun. 25, 2026

(54) DNAZYME FOR EOSINOPHIL PEROXIDASE AND USES THEREOF

(71) Applicant: McMaster University, Hamilton (CA)

(72) Inventors: Monsur Ali, Hamilton (CA); Manali Mukherjee, Hamilton (CA); Alfredo Capretta, Dundas (CA); Parameswaran Nair, Dundas (CA); John Brennan, Dundas (CA)

(21) Appl. No.: 19/128,630

(22) PCT Filed: Nov. 9, 2023
(86) PCT No.: PCT/CA2023/051496

§ 371 Date: May 9, 2025

Related U.S. Application Data
(60) Provisional application No. 63/423,930, filed on Nov. 9, 2022.

(51) Int. Cl. C12Q 1/6834

(52) U.S. Cl. CPC C12Q 1/6834

(57) ABSTRACT

This disclosure relates to catalytic nucleic acids, catalytic nucleic acid probes, biosensors, lateral flow devices, and kits for detecting the presence or absence of eosinophil peroxidase. Also provided are methods for detecting the presence or absence of eosinophil peroxidase in a test sample, using the catalytic nucleic acids, catalytic nucleic acid probes, biosensors, lateral flow devices, and kits.

### CROSS-REFERENCE TO RELATED APPLICATION

This application claims the priority and benefit of U.S. Provisional Patent Application No. 63/423,930 filed Nov. 9, 2022, herein incorporated by reference in its entirety.

### INCORPORATION OF SEQUENCE LISTING

A computer readable form of the Sequence Listing “3244-P70061PC00_SequenceListing” (89,510 bytes) was created on Oct. 29, 2023, is filed herewith by electronic submission and is incorporated by reference herein.

### FIELD

The present disclosure relates to the field of functional nucleic acids, and in particular, to DNAzymes, DNAzyme-based biosensors and methods of use thereof for detecting proteins, specifically eosinophil peroxidase.

### BACKGROUND

Functional nucleic acids (DNA or RNA aptamers, ribozymes or DNAzymes) have been well established as versatile molecular recognition elements for a variety of biosensing applications. In particular, DNA aptamers (DNA molecules that fold into unique secondary and tertiary structures to bind to specific targets with high specificity and sensitivity) are now well developed and can be selected in vitro for any type of target (small molecules, proteins, cells, etc.). However, DNAzymes, which are a special class of functional nucleic acid molecules that possess catalytic activities, have been much less developed as recognition elements. Although the first RNA-cleaving DNAzyme (RCD), which was activated by binding of Pb²⁺, was reported by Joyce and Breaker in 1994,¹ all subsequently reported RCDs have been activated by a narrow range of targets, including a selection of 10 different monovalent, divalent or trivalent metal ions, two different small molecules (adenosine and L-histidine) and a total of 7 bacterial cells and 1 mammalian cell (based on binding to mostly unknown intracellular or extracellular components).

U.S. Pat. No. 9,624,551 discloses an RCD-based probe for detecting an unidentified protein target from a microorganism.

Attempts to expand the repertoire of targets for RCDs generally involve modifying an existing metal ion-dependent RCD or its substrate to impart additional selectivity. For example, RCDs that are inactivated though methylation can be reactivated via the action of demethylases or DNA repair enzymes. Telomerase detecting RCDs have been developed where telomerase extends a DNAzyme substrate so it can hybridize to the RCD to allow cleavage. Another strategy to develop protein-selective RCDs is through rational design, wherein a protein-specific aptamer is combined with a metal-ion dependent RCD or its substrate in a manner that can modulate RCD activity. Examples include blocking of a RCD, or a DNAzyme substrate, with an aptamer, which undergoes a conformational change on protein-binding to unblock the RCD or substrate and allow the cleavage reaction to proceed; using an aptamer assisted ligation assay to alter the conformation and thus the activity of an RCD; or using aptamer-assisted assembly of a multicomponent nucleic acid enzyme (MNAzyme). However, these methods require extensive knowledge of the aptamer secondary structure and significant trial and error to design appropriate nucleic acid constructs, and hence there are currently a total of only 4 examples of a protein-selective RCDs developed by rational design.²⁻⁶

Given the importance of proteins as biomarkers for clinical diagnosis, the development of highly selective protein-activated RCDs would represent a major advance in the development of molecular recognition elements.

The background herein is included solely to explain the context of the disclosure. This is not to be taken as an admission that any of the material referred to was published, known, or part of the common general knowledge as of the priority date.

### SUMMARY

In accordance with an aspect of the present disclosure, provided herein is a catalytic nucleic acid for detecting eosinophil peroxidase comprising a nucleic acid sequence selected from the group consisting of SEQ ID NOs: 10, 12, 14, 16, 24, 25, 27-29, 33-40, 42, 43, 48, 49, 51-53, 57-64, 66, 67, 70, 71, 76, 80, and 81, a functional fragment thereof, or a functional variant thereof. In some embodiments, the nucleic acid sequence is selected from the group consisting of SEQ ID NO: 10, 12, 14, 16, 25, 27, 29, 38, 40, 43, 70, 71, 76, 80, and 81. In some embodiments, the nucleic acid sequence is SEQ ID NO: 10 or 14. In some embodiments, the catalytic nucleic acid detects eosinophil peroxidase with a limit of detection (LOD) of about 3 nM. In some embodiments, the catalytic nucleic acid catalyzes cleavage of a substrate nucleic acid.

Also provided is a catalytic nucleic acid probe comprising the catalytic nucleic acid described herein and a detectable substrate. In some embodiments, the detectable substrate comprises a ribonucleotide flanked by a fluorophore-modified nucleic acid residue and a quencher-modified nucleic acid residue.

Also provided is a biosensor for detecting eosinophil peroxidase comprising the catalytic nucleic acid described herein, or the catalytic nucleic acid probe described herein, functionalized on and/or in a material. In some embodiments, the biosensor is a lateral flow device. In some embodiments, the biosensor is for use in screening, diagnostics, guiding therapy, health monitoring, and/or pharmaceutical development. In some embodiments, the biosensor is for use in screening, diagnostics, and/or health monitoring.

Also provided is a lateral flow device for detecting the presence or absence of eosinophil peroxidase in a test sample comprising:
a) a sample pad, for applying i) a mixture comprising the test sample, the catalytic nucleic acid described herein immobilized to a solid support, and a running buffer, or ii) for applying a mixture comprising the test sample, a bridging region released from an immobilized activated catalytic nucleic acid described herein, and a running buffer, to initiate a lateral flow process, wherein the catalytic nucleic acid comprises a bridging region that is configured for release upon contacting the eosinophil peroxidase, and wherein the bridging region comprises a reagent zone test oligonucleotide binding domain configured for binding to a reagent zone test oligonucleotide by complementarity and a sensor zone test oligonucleotide binding domain configured for binding to a sensor zone test oligonucleotide by complementarity,
wherein the running buffer comprises a quenching buffer, optionally the quenching buffer comprises a denaturant, optionally an anionic denaturant, optionally the quenching buffer further comprises pullulan,
b) a reagent zone adjacent to the sample pad, the reagent zone comprising a reagent zone test oligonucleotide coupled to a nanoparticle and a reagent zone control oligonucleotide coupled to a nanoparticle, wherein the reagent zone control oligonucleotide is configured for binding to a sensor zone control oligonucleotide by complementarity,
c) a sensor zone adjacent to the reagent zone, the sensor zone comprising an immobilized sensor zone test oligonucleotide at a test line, and an immobilized sensor zone control oligonucleotide at a control line, and
d) optionally an absorbent pad,
wherein a signal produced at the test line and a signal produced at the control line indicate the presence of eosinophil peroxidase, and
wherein a signal is not produced at the test line and a signal is produced at the control line indicate the absence of eosinophil peroxidase.

In some embodiments, the catalytic nucleic acid comprises the nucleic acid sequence of SEQ ID NO: 43, 71, or 73, the reagent zone test oligonucleotide comprises the nucleic acid sequence of SEQ ID NO: 20 or 72, the sensor zone test oligonucleotide comprises the nucleic acid sequence of SEQ ID NO: 21 or 73, the reagent zone control oligonucleotide comprises the nucleic acid sequence of SEQ ID NO: 22 or 74, and the sensor zone test oligonucleotide comprises the nucleic acid sequence of SEQ ID NO: 23 or 75. In some embodiments, the solid support comprises agarose beads. In some embodiments, the catalytic nucleic acid is immobilized to the agarose beads by biotin-streptavidin interaction. In some embodiments, the lateral flow device comprises nitrocellulose paper, a polymer support layer and a hydrophobic material. In some embodiments, the nanoparticle is a gold nanoparticle. In some embodiments, the sensor zone test oligonucleotide and the sensor zone control oligonucleotide are immobilized on a paper. In some embodiments, the paper is nitrocellulose paper.

Also provided is a method for detecting the presence or absence of eosinophil peroxidase in a test sample from a subject, the method comprising:
a) applying the test sample to the catalytic nucleic acid probe described herein;
b) detecting a fluorescence signal;
wherein detecting a fluorescence signal above a specified threshold value indicates presence of eosinophil peroxidase in the test sample.

In some embodiments, the method further comprising diluting the test sample in a buffer before step a). In some embodiments, the test sample is a sputum sample. In some embodiments, the buffer comprises about 2 mM dithiothreitol. In some embodiments, the method detects airway eosinophilia in the subject.

Also provided is a method for detecting the presence or absence of eosinophil peroxidase in a test sample from a subject, the method comprising:
applying the test sample to the biosensor described herein;
detecting a colorimetric signal in a test zone;
wherein detecting a colorimetric signal in a test zone indicates the presence of eosinophil peroxidase in the test sample.

In some embodiments, the method further comprising diluting the test sample in buffer before step a). In some embodiments, the test sample is a sputum sample. In some embodiments, the buffer comprises about 2 mM dithiothreitol. In some embodiments, the method detects airway eosinophilia in the subject.

Also provided is a method of detecting the presence or absence of eosinophil peroxidase in a test sample from a subject, comprising:
applying i) a mixture comprising the test sample, the catalytic nucleic acid described herein immobilized to a solid support, and a running buffer, or ii) a mixture comprising the test sample, a bridging region released from an immobilized activated catalytic nucleic acid described herein, and a running buffer, to the sample pad of the lateral flow device described herein,
wherein the catalytic nucleic acid comprises a substrate comprising a ribonucleotide cleavage site,
wherein a quenching buffer is added to the running buffer to quench activity of the catalytic nucleic acid prior to applying the mixture to the sample pad, optionally the quenching buffer comprises a denaturant, optionally an anionic denaturant, optionally the quenching buffer further comprises pullulan,
a) wherein if the test sample comprises eosinophil peroxidase,
   i) contacting the eosinophil peroxidase with the immobilized catalytic nucleic acid, and activating the catalytic nucleic acid which cleaves the substrate at the ribonucleotide cleavage site and releases a bridging region comprising the reagent zone test oligonucleotide binding domain and the sensor zone test oligonucleotide binding domain,
   ii) allowing the running buffer and the released bridging region to laterally flow into the reagent zone, wherein a complex is formed between the bridging region and the reagent zone test oligonucleotide,
   iii) allowing the running buffer, the complex, and the reagent zone control oligonucleotide to laterally flow to the sensor zone,
   iv) allowing the complex to be captured by the sensor zone test oligonucleotide to produce a signal at a test line,
   v) detecting the signal in iv) in the sensor zone at the test line, optionally the signal is a color change signal,
   vi) allowing the reagent zone control oligonucleotide to be captured by the sensor zone control oligonucleotide to produce a signal at a control line, and
   vii) detecting the signal in vi) in the sensor zone at a control line, optionally the signal is a color change signal,
   whereby the detection of signals in both v) and vii) is indicative of the presence of eosinophil peroxidase and the correct functioning of the lateral flow device;
or
b) wherein if the test sample does not comprise eosinophil peroxidase,
   the immobilized catalytic nucleic acid is not activated and no bridging region is released,
   i) allowing the running buffer to laterally flow into the reagent zone, and then the running buffer and the reagent zone control oligonucleotide to laterally flow to the sensor zone,
   ii) detecting no signal in the sensor zone at the test line,
   iii) allowing the reagent zone control oligonucleotide to be captured by the sensor zone control oligonucleotide to produce a signal, and
   iv) detecting the signal in the sensor zone at a control line, optionally the signal is a color change signal,
   whereby the detection of no signal in ii) and the detection of the signal in iv) are indicative of the absence of eosinophil peroxidase and the correct functioning of the lateral flow device.

In some embodiments, the method comprises incubating the mixture comprising the test sample, the catalytic nucleic acid described herein which is immobilized to a solid support, and the running buffer, prior to applying the mixture or a portion thereof to the sample pad. In some embodiments, the test sample comprises a clinical sample. In some embodiments, the test sample comprises a sputum sample. In some embodiments, the method detects airway eosinophilia in the subject.

Also provided is a kit for detecting eosinophil peroxidase in a test sample, wherein the kit comprises the catalytic nucleic acid probe described herein, the biosensor described herein, the lateral flow device described herein, or components required for a method for detecting the presence or absence of eosinophil peroxidase described herein, and instructions for use of the kit.

Other features and advantages of the present disclosure will become apparent from the following detailed description. It should be understood, however, that the detailed description and the specific examples, while indicating embodiments of the disclosure, are given by way of illustration only and the scope of the claims should not be limited by these embodiments, but should be given the broadest interpretation consistent with the description as a whole.

### DRAWINGS

Certain embodiments of the disclosure will now be described in greater detail with reference to the attached drawings in which:

FIG. 1A shows a schematic of the in vitro selection of the DNAzyme for EPX protein including negative selection and positive selection steps in an exemplary embodiment of the disclosure.

FIG. 1B shows the DNA library, primers, substrate and related sequences used in the selection, wherein N50 denotes 50 random nucleotides with 25% probability of each of the 4 nucleotides (A, T, G, C) and the fixed sequence domains in both sides of N50 serve as templates for the PCR amplification in exemplary embodiments of the disclosure. DL: DNA library (SEQ ID NO: 1); FP: Forward primer (SEQ ID NO: 2); RP1: reverse primer in first PCR (SEQ ID NO: 3); RP2 reverse primer in 2^{nd} PCR (N-terminal SEQ ID NO: 4 and C-terminal SEQ ID NO: 68, linked through a triethylene glycol spacer (iSp9); LT: Ligation template (SEQ ID NO: 7); FS: substrate (SEQ ID NO: 6).

FIG. 1C shows composite image 10% dPAGE of different selection rounds showing the progress of selection (NS: negative selection, PS: positive selection) in exemplary embodiments of the disclosure. Vertical lines demarcate different gels.

FIG. 1D shows sequence truncation of the parent DNAzyme named EPDz20 (the dashes denote the deleted bases) in an exemplary embodiment of the disclosure. The tilde (˜) denotes deleted bases referenced to EPDz20. EPDz20—SEQ ID NO: 10; EPDz20M1—SEQ ID NO: 11; EPDz20M2—SEQ ID NO: 12; EPDz20M3—SEQ ID NO: 13; EPDz20M4—SEQ ID NO: 14; and EPDz20M5—SEQ ID NO: 15.

FIG. 1E shows composite image of 10% dPAGE of the cleavage reactions of the truncated sequences using 50 nM EPX and a 30 min reaction time, in exemplary embodiments of the disclosure.

FIG. 2A shows specificity of EPDz20M4 against different relevant proteins based on percent cleavage using 50 nM protein and a 30 min reaction time, in an exemplary embodiment of the disclosure. Inset shows composite image of 10% dPAGE gels where the vertical lines demarcate different gels.

FIG. 2B shows cleavage of EPDz20M4 with different concentrations of EPX in buffer in an exemplary embodiment of the disclosure. Error bars are ±3σ of the mean (n=3); error for blank is within the symbol.

FIG. 2C shows fluorescence signalling of EPDZ20M4 in the presence of EPX and individual non-target proteins (50 nM each) in exemplary embodiments of the disclosure.

FIG. 2D fluorescence signalling of EPDZ20M4 in the presence of different concentrations of EPX in selection buffer, in exemplary embodiments of the disclosure. dPAGE data is collected at 90 min reaction time.

FIG. 3A shows a schematic illustration of the LFD showing the components assembled together as a ready-to-use device in an exemplary embodiment of the disclosure. GNP=gold nanoparticle.

FIG. 3B shows the sequences used in the LFD experiments in exemplary embodiments of the disclosure. BNA-FS-EPDz20M4—SEQ ID NO: 43; TGNP-DNA—SEQ ID NO: 20; TL-DNA—SEQ ID NO: 21; CGNP-DNA—SEQ ID NO: 22; CL-DNA—SEQ ID NO: 23.

FIG. 3C shows a schematic illustration of the cleavage reaction of the DNAzyme on beads to release the bridging sequence for the LFD in an exemplary embodiment of the disclosure.

FIG. 3D shows the interpretation of the results: i) a signal for both the test line (TL) and the control line (CL) indicates a positive result, ii) if only the control line (CL) produces a signal, the result is considered to be negative, in exemplary embodiments of the disclosure.

FIG. 3E shows a 10% dPAGE image of the cleavage reactions of the BNA-FS-EPDz20M4 using 50 nM protein and a 30 min reaction time in exemplary embodiments of the disclosure.

FIG. 3F shows a specificity test of the LFD in buffer with 50 nM protein and a 30 min reaction time and 15 min elution time (inset: fluorescence images of the beads in tubes after cleavage and running on the LFD) in an exemplary embodiment of the disclosure. Error bars are ±3σ of the mean (n=3).

FIG. 3G shows a sensitivity test in buffer using a 30 min cleavage time and 15 min elution time (inset: fluorescence images of the beads in tubes after cleavage and running on the LFD) in an exemplary embodiment of the disclosure.

FIG. 4A shows a schematic of the sample processing steps for patient sputum plugs in exemplary embodiments of the disclosure.

FIG. 4B shows LFD images after running with reaction mixtures of BNA-FS-EPDz20M4 in the presence of processed patient sputum samples with a 15 min cleavage time and 15 min elution time, in exemplary embodiments of the disclosure.

FIG. 4C shows bar graph prepared from the quantified intensities of the LFDs in FIG. 4B, with a cut-off value of 3.3 based on 3σ of the mean of the negative sample intensity values, in exemplary embodiments of the disclosure. This cut-off was used to produce clinical sensitivity and specificity values.

FIG. 5A shows scatter plots showing distribution of test line intensity for eosinophil peroxidase (EPX) detection using the DNAzyme LFD for eosinophilic, mixed granulocytic (evidence of eosinophilia and neutrophils), non-eosinophilic (negative) and samples from healthy donors in exemplary embodiments of the disclosure. Negative samples were confirmed by absence of eosinophils and free eosinophil granules using a gold standard assay (routine sputum cytometry). Dotted line is lower cut-off 90^{th} percentile of healthy donors.

FIG. 5B shows the correlation between DNAzyme LFD test line intensity values with percent eosinophils present in sputum samples in an exemplary embodiment of the disclosure. Individual square symbols indicate samples with zero eosinophils but moderate to many FEGs. Each symbol is representative of one patient/individual value. Kruskal Wallis with Dunn's multiple comparison test and Spearman correlation test. P<0.05 is considered as significant.

FIG. 5C shows EPX values assessed with a standard ELISA in an exemplary embodiment of the disclosure. Each symbol is representative of one patient/individual value. Kruskal Wallis with Dunn's multiple comparison test and Spearman correlation test. P<0.05 is considered as significant.

FIG. 5D shows OD values obtained using an aptamer-based pull-down assay in an exemplary embodiment of the disclosure. Each symbol is representative of one patient/individual value. Kruskal Wallis with Dunn's multiple comparison test and Spearman correlation test. P<0.05 is considered as significant.

FIG. 6A shows a schematic illustration of structure switching construct and anticipated positive and negative tests in the presence and in absence of EPX in an exemplary embodiment of the disclosure.

FIG. 6B shows sequences used to produce an aptamer-based LFD in an exemplary embodiment of the disclosure. EPX-APT5BT—SEQ ID NO: 44; EPX-BA—SEQ ID NO: 45; TGNP-DNA—SEQ ID NO: 20; TL-DNA—SEQ ID NO: 21; CGNP-DNA—SEQ ID NO: 46; CL-DNA—SEQ ID NO: 47.

FIG. 6C shows LFD images obtained from positive and negative patient sputum samples without added SDS, in an exemplary embodiment of the disclosure. NC: negative control (buffer alone), N6: EOS negative sputum sample, P5: EOS positive sputum sample. The negative sputum sample shows background on the test line.

FIG. 6D shows LFD images in the presence of SDS in an exemplary embodiment of the disclosure. N6: EOS negative sputum sample, P5: EOS positive sputum sample showing lack of a test line.

FIG. 7 shows the unprocessed full gel images used in FIG. 1C. The arrow indicates gel images were used for NS or PS in the rounds as labelled in exemplary embodiments of the disclosure.

FIG. 8A shows the top 20 sequences obtained from the sequences of round 15 population in exemplary embodiments of the disclosure. Sequences for EPDz01 (SEQ ID NO: 48), EPDz02 (SEQ ID NO: 49), EPDz03 (SEQ ID NO: 50), EPDz04 (SEQ ID NO: 51), EPDz05 (SEQ ID NO: 52), EPDz06 (SEQ ID NO: 53), EPDz07 (SEQ ID NO: 54), EPDz08 (SEQ ID NO: 55), EPDz09 (SEQ ID NO: 56), EPDz10 (SEQ ID NO: 57), EPDz11 (SEQ ID NO: 58), EPDz12 (SEQ ID NO: 59), EPDz13 (SEQ ID NO: 60), EPDz14 (SEQ ID NO: 61), EPDz15 (SEQ ID NO: 62), EPDz16 (SEQ ID NO: 63), EPDz17 (SEQ ID NO: 64), EPDz18 (SEQ ID NO: 65), EPDz19 (SEQ ID NO: 66), and EPDz20 (SEQ ID NO: 67), are shown.

FIG. 8B shows 10% dPAGE images obtained from the cleavage reactions (30 min cleavage time) of the sequences of FIG. 8A along with percent cleavage (DNAzymes without a percent cleavage were at 0%) in exemplary embodiments of the disclosure. SB: selection buffer, EPX: eosinophilic peroxidase, Unclv: Uncleaved full length sequences, Clv: Cleaved fragments of the reaction.

FIG. 9 shows unprocessed full gel images used to produce FIG. 1E as labelled in exemplary embodiments of the disclosure.

FIG. 10A shows the predicted secondary structures of EPDz20 ligated to the substrate FS (SEQ ID NO: 80) in an exemplary embodiment of the disclosure.

FIG. 10B shows the predicted secondary structurers of EPDz20M4 ligated to the substrate FS (SEQ ID NO: 81) in an exemplary embodiment of the disclosure.

FIG. 11 shows a 10% dPAGE image for the cleavage reactions of EPDz20M4 with different salt conditions in exemplary embodiments of the disclosure. Lane 1) 50 mM HEPES, 150 mM NaCl, 15 mM MgCl2, 0.01% Tween20, pH 7.5. Lane 2) 50 mM HEPES, 150 mM NaCl, 0.01% Tween20, pH 7.5. Lane 3) 50 mM HEPES, 150 mM NaCl, 15 mM BaCl₂, 0.01% Tween20, pH 7.5. Lane 4) 50 mM HEPES, 50 mM NaCl, 15 mM MgCl₂, 0.01% Tween20, pH 7.5. Lane 5) 50 mM HEPES, 150 mM NaCl, 15 mM CaCl₂, 0.01% Tween20, pH 7.5. Lane 6) 50 mM HEPES, 150 mM NaCl, 15 mM MnCl₂, 0.01% Tween20, pH 7.5. Lane 7) 50 mM Tris-HCl, 150 mM NaCl, 15 mM MgCl₂, 0.01% Tween20, pH 7.5).

FIG. 12 shows the unprocessed full gel images used to produce FIG. 2A as labelled in exemplary embodiments of the disclosure.

FIG. 13 shows the nuclease resistance tests in exemplary embodiments of the disclosure. NC: negative control where only buffer was used instead of any nuclease. Experimental details are described in the Examples.

FIG. 14 shows a 10% dPAGE image for the cleavage reactions of EPDz20M4 with hemin and hemoglobin S in exemplary embodiments of the disclosure.

FIG. 15 shows a 10% dPAGE image for the cleavage reactions of EPDz20M4 with different versions of EPX obtained from different sources in exemplary embodiments of the disclosure. Lee Bio and Fritzerald EPXs were with full length versions isolated from human blood showing high peroxidase activity and the Sigma and LSBIO versions were human recombinant EPX other two were shorter fragments (recombinant versions).

FIG. 16 shows a 10% dPAGE image for the cleavage reactions of EPDz20M4 with sputum samples without and with EPX added in exemplary embodiments of the disclosure. All sputum samples were processed identically to samples for LFD testing and all were tested to be negative for EOS using the gold-standard assay.

FIG. 17 shows fluorescence signalling of BNA-FS-EPDz20M4 in the presence of an EOS positive sputum sample, an EOS negative sputum sample and 1×SB buffer in exemplary embodiments of the disclosure.

FIG. 18A shows a 10% dPAGE image for the cleavage reactions of BNA-FS-EPDz20M4 with 40% of the processed sputum samples (Unclv: uncleaved full length BNA-FS-EPDz20M4, Clv: Cleaved fluorescein containing fragment of BNA-FS-EPDz20M4) in exemplary embodiments of the disclosure. Processed samples were already 8-fold diluted.

FIG. 18B shows LFD images after running with the reaction mixtures from FIG. 18A in exemplary embodiments of the disclosure.

FIG. 19A shows LFD images after running with the reaction mixtures without pullulan containing buffer in exemplary embodiments of the disclosure.

FIG. 19B shows LFD images after running with the reaction mixtures with (0.5%) pullulan containing buffer in exemplary embodiments of the disclosure.

FIG. 20 shows in the top row LFD images after running 15 min and in the bottom row LFD images after sitting overnight at room temperature, in an exemplary embodiment of the disclosure.

FIG. 21A shows fluorescence images of the reaction tubes for a subset of 27 samples following LFD analysis in exemplary embodiments of the disclosure.

FIG. 21B shows bar graphs obtained from the fluorescence of the tubes of FIG. 21A in exemplary embodiments of the disclosure. Taking the cut-off value 25 produces 100% specificity and sensitivity.

FIG. 22A shows the images of the representative LFDs with different samples as labelled in an exemplary embodiment of the disclosure.

FIG. 22B shows the bar graphs obtained from the signal intensities of TL of each LFD tested with sputum samples as labelled in an exemplary embodiment of the disclosure. The error bars were obtained from two independent experiments.

FIG. 23 shows the correlation between sputum eosinophils and LFD test line signal intensity in exemplary embodiments of the disclosure.

### DETAILED DESCRIPTION

### I. Definitions

Unless otherwise indicated, the definitions and embodiments described in this and other sections are intended to be applicable to all embodiments and aspects of the present disclosure herein described for which they are suitable as would be understood by a person skilled in the art. It is also to be understood that the terminology used herein is for the purpose of describing particular aspects only and is not intended to be limiting.

In understanding the scope of the present disclosure, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. The term “consisting” and its derivatives, as used herein, are intended to be closed terms that specify the presence of the stated features, elements, components, groups, integers, and/or steps, but exclude the presence of other unstated features, elements, components, groups, integers and/or steps. The term “consisting essentially of”, as used herein, is intended to specify the presence of the stated features, elements, components, groups, integers, and/or steps as well as those that do not materially affect the basic and novel characteristic(s) of features, elements, components, groups, integers, and/or steps.

Terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies. The modifier “about” used in connection with a quantity is inclusive of the stated value and has the meaning dictated by the context (e.g., it includes the degree of error associated with measurement of the particular quantity). In addition, all ranges disclosed herein are inclusive of the endpoints and also any intermediate range points, whether explicitly stated or not, and the endpoints are independently combinable with each other.

As used in this disclosure, the singular forms “a”, “an” and “the” include plural references unless the content clearly dictates otherwise.

In embodiments comprising an “additional” or “second” component, the second component as used herein is chemically different from the other components or first component. A “third” component is different from the other, first, and second components, and further enumerated or “additional” components are similarly different.

The term “and/or” as used herein means that the listed items are present, or used, individually or in combination. In effect, this term means that “at least one of” or “one or more” of the listed items is used or present.

The abbreviation, “e.g.” is derived from the Latin exempli gratia and is used herein to indicate a non-limiting example. Thus, the abbreviation “e.g.” is synonymous with the term “for example.” The word “or” is intended to include “and” unless the context clearly indicates otherwise.

The term “sample” or “test sample” as used herein refers to any material in which the presence, absence, or amount of a target analyte is unknown and can be determined in an assay. The sample can be from any source, for example, any biological (e.g. human or animal samples, including clinical samples), environmental (e.g. water, soil or air) or natural (e.g. plants) source, or from any manufactured or synthetic source (e.g. food or drinks). The sample can be comprised or is suspected of comprising one or more analytes. The sample can be a “biological sample” comprising cellular and non-cellular material, including, but not limited to, tissue samples, saliva, sputum, urine, blood, serum, other bodily fluids and/or secretions. In some embodiments, the sample comprises saliva, sputum, oropharyngeal and/or nasopharyngeal secretions. In some embodiments, the sample comprises sputum. In some embodiments, the test sample comprises a sputum sample.

The term “target”, “analyte” or “target analyte” as used herein refer to any agent, including, but not limited to, a small inorganic molecule, small organic molecule, metal ion, biomolecule, toxin, biopolymer (such as a nucleic acid, carbohydrate, lipid, peptide, protein), cell, tissue, microorganism and virus, for which one would like to sense or detect. The analyte can be either isolated from a natural source or synthetic. The analyte can be a single compound or a class of compounds, such as a class of compounds that share structural or functional features. The term analyte also includes combinations (e.g. mixtures) of compounds or agents such as, but not limited, to combinatorial libraries and samples from an organism or a natural environment.

The term “treatment or treating” as used herein refer to an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, and remission (whether partial or total), whether detectable or undetectable.

The term “subject” as used herein includes all members of the animal kingdom including mammals such as a mouse, a rat, a dog and a human.

The term “nucleic acid” as used herein refers to a biopolymer comprising monomers of nucleotides, such as deoxyribonucleic acid (DNA), ribonucleic acid (RNA) and other polynucleotides of modified nucleotides and/or nucleotide derivatives, and can be either double stranded (ds) or single stranded (ss). In some embodiments, modified nucleotides contain one or more modified bases (e.g. unusual bases such as inosine, and functional modifications to the bases such as amino), modified backbones (e.g. peptide nucleic acid, PNA) and/or other chemically, enzymatically, or metabolically modified forms. Examples of modified nucleotides which can be used to generate the nucleic acids disclosed herein include xanthine, hypoxanthine, 2-aminoadenine, 6-methyl, 2-propyl and other alkyl adenines, 5-halo uracil, 5-halo cytosine, 6-aza uracil, 6-aza cytosine and 6-aza thymine, pseudo uracil, 4-thiouracil, 8-halo adenine, 8-aminoadenine, 8-thiol adenine, 8-thiolalkyl adenines, 8-hydroxyl adenine and other 8-substituted adenines, 8-halo guanines, 8 amino guanine, 8-thiol guanine, 8-thiolalkyl guanines, 8-hydroxyl guanine and other 8-substituted guanines, other aza and deaza uracils, thymidines, cytosines, adenines, or guanines, 5-trifluoromethyl uracil and 5-trifluoro cytosine or fluorophore and quencher conjugated nucleotides. Alternatively, the nucleic acid molecules can be produced biologically using an expression vector. In some embodiments, modified nucleotides comprise one or more modified bases (e.g. unusual bases such as inosine, and functional modifications to the bases such as amino modifications), modified backbones (e.g. peptide nucleic acid, PNA) and/or other chemically, enzymatically, or metabolically modified forms. The term “functional fragment” as used herein refers to a fragment of the nucleic acid that retains the functional property of the full-length nucleic acid, for example, the ability of the fragment to act as a DNAzyme for detecting a particular analyte, for example, eosinophil peroxidase. In some embodiments, modified nucleotides contain one or more modified bases (e.g. unusual bases such as inosine, and functional modifications to the bases such as amino), modified backbones (e.g. peptide nucleic acid, PNA) and/or other chemically, enzymatically, or metabolically modified forms.

The term “catalytic nucleic acid”, “catalytic DNA”, “deoxyribozyme”, “DNA enzyme”, or “DNAzyme” as used herein can refer to a nucleic acid molecule or oligonucleotide sequence that can catalyze or initiate a reaction. DNAzymes can be single-stranded DNA and can include RNA, modified nucleotides and/or nucleotide derivatives. In some embodiments, the DNAzyme is “RNA-cleaving” and catalyzes the cleavage of a particular substrate, for example a nucleic acid sequence comprising one or more ribonucleotides, at a defined cleavage site. In some embodiments, the substrate is a target nucleic acid in a test sample. In some embodiments, the DNAzyme cleaves a single ribonucleotide linkage. In some embodiments, the single ribonucleotide linkage is in a nucleic acid sequence wherein the remaining nucleotides are ribonucleotides. In some embodiments, the single ribonucleotide linkage is in a nucleic acid sequence wherein the remaining nucleotides are deoxyribonucleotides. In some embodiments, the DNAzyme cleaves a nucleic acid sequence at a single ribonucleotide linkage thereby producing a nucleic acid cleavage fragment.

The term “hybridizes”, “hybridized” or “hybridization” as used herein refers to the sequence specific non-covalent binding interaction with a complementary, or partially complementary, nucleic acid sequence. When, for example, the 5′-end region of an aptamer hybridizes to the 3′-end region, it can form a duplex DNA element.

The term “biosensor” as used herein refers to a device that incorporates a biological entity as a molecular recognition element and is capable of producing a measurable signal upon binding of a target analyte to the molecular recognition element. The biosensor can also be part of a larger device.

The term “functionalizing” or “functionalized on” as used herein refers to various common approaches for functionalizing a material, which can be classified as mechanical, physical, chemical and biological. Any suitable form of coupling can be utilized (e.g. coating, binding, etc.). The functionalized material, for example, a nucleic acid or a blocking species, is also immobilized.

The term “lateral flow device” as used herein refers to a device that includes one or more fluid channels, chambers or conduits that spontaneously drive a fluid across the device (e.g. by capillary force). A lateral flow device is well known in the art and it can be in a variety of formats designed by the person skilled in the art, including sandwich, competitive, and multiplex. A lateral flow device can also be used with a variety of affinity reagents, such as antibodies and aptamers (see e.g., Sajid M, Kawde A and Daud M (2015) Design, Formats and Applications of Lateral Flow Assays, J Saudi Chem Soc., 19, 689-705, and Bahadιr EB & Sezgintürk MK (2016) Lateral flow assays: Principles, designs and labels. Trends in Analytical Chemistry, 82, 286-306, the contents of which are incorporated by reference herein in their entireties).

The term “anionic denaturant”, as used herein, refers to a type of surfactant, specifically denaturing agents that have negatively charged functional groups, which confer anionic properties in solution. These denaturants primarily function to disrupt the native three-dimensional structures of biomolecules, particularly proteins and nucleic acids, through a combination of ionic interactions and alteration of solvation dynamics. The anionic characteristic is typically derived from the presence of functional groups such as sulfates, sulfonates, or carboxylates, which become ionized under physiological or experimental conditions. This ionization facilitates the disruption of non-covalent interactions within biomolecules, leading to denaturation. Examples of anionic denaturants include, but are not limited to, sodium dodecyl sulfate (SDS), sodium deoxycholate, sodium cholate, sodium lauroyl sarcosinate, sodium caprylate, sodium N-lauroylsarcosine, lithium dodecyl sulfate, and sodium N-undecylenoyl sarcosinate. These agents are widely used in biochemical and molecular biology applications for purposes such as protein solubilization, disruption of protein complexes, and preparation of samples for electrophoretic analyses and other assays.

It will be understood that any component defined herein as being included can be explicitly excluded by way of proviso or negative limitation, such as any specific compounds or method steps, whether implicitly or explicitly defined herein.

Although methods and materials similar or equivalent to those described herein can be used in the practice or testing of this disclosure, suitable methods and materials are described below.

### II. DNAzymes, Biosensors, Kits, and Methods of the Disclosure

Disclosed herein is an RNA-cleaving DNAzyme (RCD) selected against the protein target eosinophil peroxidase (EPX, sometimes denoted as EPO), a validated marker for airway eosinophilia, which plays a key role in the pathogenesis of asthma, chronic bronchitis and other airway diseases and can be used as a marker to guide therapy. This target is particularly challenging as autoimmune responses to eosinophil degranulation products, including EPX, can produce high polyclonal IgG levels in the airways, significantly impacting the performance of antibody-based lateral flow devices (LFDs).

This disclosure represents the development of the first known example of a protein-activated RCD obtained directly by in vitro selection, which can detect a specific protein target of interest. Many human conditions, including heart disease, various cancers and others have highly specific protein biomarkers that can be used to enable rapid diagnostic tests. Hence, it is critical to be able to generate highly sensitive and selective molecular recognition elements (MREs) for specific protein markers. However, the generation of such allosteric RCDs is not currently possible using either the rational design approach, owing to challenges with the design of functional aptazymes, or through the use of cellular media, which produces aptazymes with unknown targets and provides no mechanism to bias the selection toward a specific protein target.

Eosinophils release many granule proteins and inflammatory mediators, of which EPX is the most specific, and therefore can be used for identifying eosinophilia. Related granule proteins, such as eosinophil cationic protein (ECP) and eosinophil-derived neurotoxin (EDN), are non-specific markers as they are also produced by neutrophils and macrophages. Myeloperoxidase (MPO), on the contrary, is highly specific to neutrophils but can interfere with EPX detection due to high sequence homology. In patients who have airway infections with high neutrophil numbers, or excessive degranulation, eosinophilia (based on an intact cellular differential of ≥3%) can often be masked, and therefore difficult to identify.

This disclosure shows that by employing positive selection with EPX and counter selection with a mixture of MPO and other relevant proteins, a sensitive and selective EPX-specific RCD can be selected. This RCD has an affinity constant for EPX in the low nanomolar range, shows high selectivity against related peroxidases and other eosinophil proteins, and is resistant to degradation by mammalian nucleases. Following improvement, the RCD was used to develop a simple fluorescence assay and lateral flow device that could detect EPX in sputum within 45 min using a simplified sample preparation protocol. These assays were validated with patient sputum samples (healthy, eosinophil negative, mixed eosinophil and neutrophil positive, and eosinophil positive), providing clinical sensitivity and specificity of 100% for the fluorescence assay, and an LFD with clinical sensitivity of 100% and specificity of 96%, thus demonstrating the potential of the protein-selective RCD for clinical diagnostic applications.

Disclosed herein is the generation of a catalytic nucleic acid generated for a specific protein target of interest. In some embodiments, the specific protein target of interest is a human protein. In some embodiments, the human protein target of interest is a marker of human health and/or disease.

Therefore, in accordance with an aspect of the present disclosure, provided herein is a catalytic nucleic acid for detecting eosinophil peroxidase comprising a nucleic acid sequence selected from the group consisting of SEQ ID NOs: 10, 12, 14, 16, 24, 25, 27-29, 33-40, 42, 43, 48, 49, 51-53, 57-64, 66, 67, 70, 71, 76, 80, and 81, a functional fragment thereof, or a functional variant thereof.

In some embodiments, the catalytic nucleic acid comprises a nucleic acid sequence selected from the group consisting of SEQ ID NOs: 10, 12, 14, 16, 24, 25, 27-29, 33-40, 42, 43, 70, 71, 76, 80, and 81, a functional fragment, or a functional variant thereof.

In some embodiments, the catalytic nucleic acid comprises the nucleic acid sequence selected from the group consisting of SEQ ID NO: 10, 12, 14, 16, 25, 27, 29, 38, 40, 43, 70, 71, 76, 80, and 81.

In some embodiments, the catalytic nucleic acid comprises the nucleic acid sequence of SEQ ID NO: 10 or 14.

In some embodiments, the catalytic nucleic acid comprises the nucleic acid sequence of SEQ ID NO: 10. In some embodiments, the catalytic nucleic acid comprises the nucleic acid sequence of SEQ ID NO: 12. In some embodiments, the catalytic nucleic acid comprises the nucleic acid sequence of SEQ ID NO: 14. In some embodiments, the catalytic nucleic acid comprises the nucleic acid sequence of SEQ ID NO: 16. In some embodiments, the catalytic nucleic acid comprises the nucleic acid sequence of SEQ ID NO: 19. In some embodiments, the catalytic nucleic acid comprises the nucleic acid sequence of SEQ ID NO: 24. In some embodiments, the catalytic nucleic acid comprises the nucleic acid sequence of SEQ ID NO: 25. In some embodiments, the catalytic nucleic acid comprises the nucleic acid sequence of SEQ ID NO: 27. In some embodiments, the catalytic nucleic acid comprises the nucleic acid sequence of SEQ ID NO: 28. In some embodiments, the catalytic nucleic acid comprises the nucleic acid sequence of SEQ ID NO: 29. In some embodiments, the catalytic nucleic acid comprises the nucleic acid sequence of SEQ ID NO: 33. In some embodiments, the catalytic nucleic acid comprises the nucleic acid sequence of SEQ ID NO: 34. In some embodiments, the catalytic nucleic acid comprises the nucleic acid sequence of SEQ ID NO: 35. In some embodiments, the catalytic nucleic acid comprises the nucleic acid sequence of SEQ ID NO: 36. In some embodiments, the catalytic nucleic acid comprises the nucleic acid sequence of SEQ ID NO: 37. In some embodiments, the catalytic nucleic acid comprises the nucleic acid sequence of SEQ ID NO: 38. In some embodiments, the catalytic nucleic acid comprises the nucleic acid sequence of SEQ ID NO: 39. In some embodiments, the catalytic nucleic acid comprises the nucleic acid sequence of SEQ ID NO: 40. In some embodiments, the catalytic nucleic acid comprises the nucleic acid sequence of SEQ ID NO: 42. In some embodiments, the catalytic nucleic acid comprises the nucleic acid sequence of SEQ ID NO: 43. In some embodiments, the catalytic nucleic acid comprises the nucleic acid sequence of SEQ ID NO: 70. In some embodiments, the catalytic nucleic acid comprises the nucleic acid sequence of SEQ ID NO: 71. In some embodiments, the catalytic nucleic acid comprises the nucleic acid sequence of SEQ ID NO: 76. In some embodiments, the catalytic nucleic acid comprises the nucleic acid sequence of SEQ ID NO: 80. In some embodiments, the catalytic nucleic acid comprises the nucleic acid sequence of SEQ ID NO: 81.

In some embodiments, the catalytic nucleic acid detects eosinophil peroxidase with at least nanomolar sensitivity. In some embodiments, the catalytic nucleic acid detects eosinophil peroxidase with a limit of detection (LOD) of about 3 nM to about 200 nM. In some embodiments, the catalytic nucleic acid detects eosinophil peroxidase with the LOD of about 3 nM.

In some embodiments, the catalytic nucleic acid catalyzes cleavage of a substrate nucleic acid.

Also provided herein is a catalytic nucleic acid probe comprising the catalytic nucleic acid described herein and a detectable substrate.

In some embodiments, the detectable substrate comprises a ribonucleotide flanked by a fluorophore-modified nucleic acid residue and a quencher-modified nucleic acid residue. In some embodiments, the detectable substrate comprises a single ribonucleotide linkage embedded in a DNA sequence and the ribonucleotide linkage is directly flanked by a fluorophore modified nucleic acid residue on one side and a quencher modified nucleic acid residue on the other side such that the fluorophore is quenched until the ribonucleotide linkage is cleaved, thereby generating a fluorogenic signal.

Provided herein is also a biosensor for detecting airway eosinophilia comprising a catalytic nucleic acid described herein functionalized on and/or in a material. In some embodiments, the biosensor comprises a nucleic acid comprising a nucleic acid sequence described herein. In some embodiments, the biosensor comprises a nucleic acid comprising a nucleic acid sequence described in Table 1, Table 2, or Table 4.

In some embodiments, the biosensor is a lateral flow device.

In some embodiments, the biosensor is for use in screening, diagnostics, guiding therapy, health monitoring, and/or pharmaceutical development. In some embodiments, the biosensor is for use in screening, diagnostics, and/or health monitoring. In some embodiments, the biosensor is for use in guiding therapy.

In some embodiments, the biosensor described herein is for use in screening, diagnostics, guiding therapy, health monitoring, and/or pharmaceutical development.

In some embodiments, the biosensor described herein is for use in screening, diagnostics, and/or health monitoring. In some embodiments, the biosensor described herein is for use in detecting eosinophil peroxidase in point-of-care diagnostics and health monitoring. In some embodiments, the biosensor described herein is for use in detecting airway eosinophilia. In some embodiments, the biosensor described herein can be used without the need for test sample pre-treatment, target labeling, and/or amplification. In some embodiments, the biosensor described herein increases the accuracy and decreases the timeline for diagnosis.

In some embodiments, the biosensor is a lateral flow device comprising a catalytic nucleic acid described herein that detects the presence or absence of eosinophil peroxidase. The lateral flow device described herein is intended for rapid detection of the presence or absence of eosinophil peroxidase in a test sample such as sputum without the need for costly or sophisticated equipment. This device is useful in point-of-care applications, as well as in laboratory testing or medical diagnostics. There are a number of configurations for a lateral flow device known to the person skilled in the art. For example, the lateral flow device described herein can comprise a reagent zone, for example, a conjugate pad, having an immobilized and stabilized catalytic nucleic acid, and a sensor zone having stabilized reporting solution. In some embodiments, the lateral flow device comprises a sample pad for applying a running buffer, optionally the test sample, optionally the catalytic nucleic acid described herein, or a mixture thereof comprising the running buffer, the test sample, and the catalytic nucleic acid, the sample pad being connected through a flow channel to ii) a reagent zone, optionally for applying a test sample, the reagent zone being connected through a flow channel to iii) a sensor zone for indicating the presence, absence, or a range of levels of an analyte. In some embodiments, the running buffer comprises one or more reagents or detection agents. In some embodiments, the sample pad is immobilized to a solid support. In some embodiments, the reagent zone is immobilized to a solid support. In some embodiments, the sensor zone is immobilized to a solid support. In some embodiments, the sensor zone comprises a plurality of testing zones. In some embodiments, the sample pad, the reagent zone, and the sensor zone are immobilized on a solid support. In some embodiments, the sample pad comprises one or more reagents or detection agents for an analyte. In some embodiments, the reagent zone comprises one or more reagents or detection agents for an analyte. In some embodiments, the sensor zone comprises one or more reagents or detection agents for an analyte. In some embodiments, the sample pad comprises one or more catalytic nucleic acids described herein. In some embodiments, the reagent zone comprises one or more reagent zone test oligonucleotides described herein. In some embodiments, the reagent zone comprises one or more reagent zone control oligonucleotides described herein. In some embodiments, the sensor zone comprises one or more sensor zone test oligonucleotides described herein. In some embodiments, the sensor zone comprises one or more sensor zone control oligonucleotides described herein. In some embodiments, the one or more catalytic nucleic acids are specific for eosinophil peroxidase. In some embodiments, the one or more catalytic nucleic acids are coupled to a detectable label. In some embodiments, the one or more reagents or detection agents are capable of reacting with the analyte and/or the one or more catalytic nucleic acid to generate a detection signal. In some embodiments, the detection signal is a visible color change.

In another embodiment, the lateral flow device comprises a sample zone and a sensor zone. In some embodiments, the sample zone and the sensor zone are connected through a flow channel. In some embodiments, the sample zone is for applying a running buffer and an analyte. In some embodiments, the sample zone is for applying a mixture comprising a running buffer and an analyte. In some embodiments, the lateral flow device comprises a sample zone for applying a mixture of a running buffer and an analyte, the sample zone being connected through a flow channel to a sensor zone for indicating the presence, absence, or a range of levels of an analyte. In some embodiments, the sample zone is immobilized to a solid support. In some embodiments, the sensor zone is immobilized to a solid support. In some embodiments, the sample zone and the sensor zone are immobilized to a solid support. In some embodiments, the sensor zone comprises a plurality of testing zones. In some embodiments, the sample zone comprises one or more reagents or detect agents for an analyte. In some embodiments, the sensor zone comprises one or more reagents or detect agents for an analyte. In some embodiments, the sample zone comprises one or more analyte-specific DNAzymes described herein. In some embodiments, the sensor zone comprises one or more analyte-specific DNAzymes described herein. In some embodiments, the sample zone and the sensor zone each comprises one or more analyte-specific DNAzymes described herein. In some embodiments, the analyte is eosinophil peroxidase. In some embodiments, the one or more analyte-specific DNAzymes are specific for eosinophil peroxidase. In some embodiments, the one or more analyte-specific DNAzymes are coupled to a detectable label. In some embodiments, the one or more reagents or detection agents are capable of reacting with the analyte and/or the one or more analyte-specific DNAzymes to generate a detection signal. In some embodiments, the detection signal is a visible color change.

A number of materials are useful for making the lateral flow device described herein. The lateral flow device can have separated zones and flow channels. Such zones and flow channels can be created by wax on a nitrocellulose paper backed with a plastic sheet, i.e. wax acts as a uniform hydrophobic barrier for which the running buffer does not penetrate and the nitrocellulose paper acts to allow lateral flow of the running buffer. Methods for creating a hydrophobic barrier on a support layer are known to the person skilled in the art. The skilled person also recognizes that many alternatives to nitrocellulose paper are possible, for example, any material that allows flow could work, such as cellulose, or any other surface that supports capillary flow. Accordingly, in some embodiments, the lateral flow device comprises nitrocellulose paper, cellulose, or any surface that supports capillary flow. In some embodiments, the lateral flow device comprises nitrocellulose paper. In some embodiments, the lateral flow device comprises a polymer support layer. In some embodiments, the polymer support layer comprises a plastic sheet. In some embodiments, the lateral flow device comprises a hydrophobic material. In some embodiments, the hydrophobic material comprises wax. In some embodiments, the lateral flow device was printed by a hydrophobic material. In some embodiments, the lateral flow device was printed by wax.

In some embodiments, the lateral flow device for detecting the presence or absence of eosinophil peroxidase in a test sample comprises:
a) a sample pad, for applying i) a mixture comprising the test sample, the catalytic nucleic acid described herein immobilized to a solid support, and a running buffer, or ii) for applying a mixture comprising the test sample, a bridging region released from an immobilized activated catalytic nucleic acid, and a running buffer, to initiate a lateral flow process,
wherein the catalytic nucleic acid comprises a bridging region that is configured for release upon contacting the eosinophil peroxidase, and wherein the bridging region comprises a reagent zone test oligonucleotide binding domain configured for binding to a reagent zone test oligonucleotide by complementarity and a sensor zone test oligonucleotide binding domain configured for binding to a sensor zone test oligonucleotide by complementarity,
wherein the running buffer comprises a quenching buffer,
b) a reagent zone adjacent to the sample pad, the reagent zone comprising a reagent zone test oligonucleotide coupled to a nanoparticle and a reagent zone control oligonucleotide coupled to a nanoparticle, wherein the reagent zone control oligonucleotide is configured for binding to a sensor zone control oligonucleotide by complementarity,
c) a sensor zone adjacent to the reagent zone, the sensor zone comprising an immobilized sensor zone test oligonucleotide at a test line, and an immobilized sensor zone control oligonucleotide at a control line, and
d) optionally an absorbent pad,
wherein a signal produced at the test line and a signal produced at the control line indicate the presence of eosinophil peroxidase, and
wherein a signal is not produced at the test line and a signal is produced at the control line indicate the absence of eosinophil peroxidase.

In some embodiments, the catalytic nucleic acid comprises the nucleic acid sequence of SEQ ID NO: 43, 71, or 73, the reagent zone test oligonucleotide comprises the nucleic acid sequence of SEQ ID NO: 20 o 72, the sensor zone test oligonucleotide comprises the nucleic acid sequence of SEQ ID NO: 21 or 73, the reagent zone control oligonucleotide comprises the nucleic acid sequence of SEQ ID NO: 22 or 74, and the sensor zone test oligonucleotide comprises the nucleic acid sequence of SEQ ID NO: 23 or 75. In some embodiments, the bridging region binds at a first end the reagent zone test oligonucleotide by complementarity and binds at a second end the the sensor zone test oligonucleotide at the test line by complementarity, wherein the reagent zone test oligonucleotide is coupled to a nanoparticle.

In some embodiments, the running buffer comprises from about 1 mM to about 3 mM dithiothreitol (DTT). In some embodiments, the running buffer comprises about 2 mM DTT. In some embodiments, DTT is for use to effectuate the breakdown of sputum. In some embodiments, catalytic activity of the catalytic nucleic acid is arrested by the quenching buffer. In some embodiments, the quenching buffer comprises a denaturant. In some embodiments, the denaturant is an anionic denaturant, a neutral denaturant, or a cationic denaturant. In some embodiments, the denaturant is for binding to protein. In some embodiments, the quenching buffer comprises an anionic denaturant. In some embodiments, the anionic denaturant comprises SDS, sodium deoxycholate, sodium cholate, sodium lauroyl sarcosinate, sodium caprylate, sodium N-lauroylsarcosine, lithium dodecyl sulfate, or sodium N-undecylenoyl sarcosinate. In some embodiments, the neutral denaturant comprises urea. In some embodiments, the cationic denaturant comprises guanidine hydrochloride. In some embodiments, the urea is at a final concentration of about 0.5 M to about 2 M. In some embodiments, the urea is at a final concentration of about 1 M. In some embodiments, the guanidine hydrochloride is at a final concentration of about 0.5 M to about 2 M. In some embodiments, the guanidine hydrochloride is at a final concentration of about 1 M. In some embodiments, the SDS is at a final concentration of about 0.1% (w/v) to about 2% (w/v). In some embodiments, the SDS is at a final concentration of about 0.1% (w/v) to about 0.5% (w/v). In some embodiments, the SDS is at a final concentration of about 0.5% (w/v). In some embodiments, the SDS is at a final concentration of about 0.25% (w/v). In some embodiments, the quenching buffer further comprises pullulan. In some embodiments, the pullulan promotes nucleic acid hybridization. In some embodiments, the pullulan is at a final concentration of about 0.1% (w/v) to about 1% (w/v). In some embodiments, the pullulan is at a final concentration of about 0.5% (w/v). In some embodiments, the quenching buffer further comprises pullulan. In some embodiments, the pullulan is at a final concentration of about 0.1% (w/v) to about 1% (w/v). In some embodiments, the pullulan is at a final concentration of about 0.5% (w/v).

In some embodiments, the solid support comprises agarose beads. In some embodiments, the catalytic nucleic acid is immobilized to the agarose beads by biotin-streptavidin interaction. In some embodiments, the lateral flow device comprises nitrocellulose paper, a polymer support layer and a hydrophobic material. In some embodiments, the nanoparticle is a gold nanoparticle. In some embodiments, the sensor zone test oligonucleotide and the sensor zone control oligonucleotide are immobilized on a paper. In some embodiments, the paper is nitrocellulose paper.

Provided herein is also a method for detecting the presence or absence of eosinophil peroxidase in a test sample from a subject, the method comprising:
a) applying the test sample to the catalytic nucleic acid probe described herein;
b) detecting a fluorescence signal;
wherein detecting a fluorescence signal above a specified threshold value indicates presence of eosinophil peroxidase in the test sample and a fluorescence signal below a specified threshold value indicates the absence of eosinophil peroxidase in the test sample.

Also provided is a method for detecting the presence or absence of eosinophil peroxidase in a test sample from a subject, the method comprising:
a) applying the test sample to the biosensor described herein;
b) detecting a colorimetric signal in a test zone;
wherein detecting a colorimetric signal in a test zone indicates the presence of eosinophil peroxidase in the test sample and a lack of a colorimetric signal in a test zone indicates the absence of eosinophil peroxidase in the test sample.

In some embodiments, the method further comprises diluting the test sample in buffer before step a). In some embodiments, the buffer provides conditions for cleavage by the catalytic nucleic acid described herein.

In some embodiments, the buffer comprises from about 1 mM to about 3 mM dithiothreitol (DTT). In some embodiments, the buffer comprises about 2 mM DTT. In some embodiments, DTT is for use to effectuate the breakdown of sputum.

In some embodiments, catalytic cleavage of a substrate nucleic acid is arrested by applying a quenching buffer to the test sample. In some embodiments, the running buffer comprises from about 1 mM to about 3 mM dithiothreitol (DTT). In some embodiments, the running buffer comprises about 2 mM DTT. In some embodiments, DTT is for use to effectuate the breakdown of sputum in the sputum sample. In some embodiments, catalytic activity of the catalytic nucleic acid is arrested by the quenching buffer. In some embodiments, the quenching buffer comprises a denaturant. In some embodiments, the denaturant is an anionic denaturant, a neutral denaturant, or a cationic denaturant. In some embodiments, the denaturant is for binding to protein. In some embodiments, the quenching buffer comprises an anionic denaturant. In some embodiments, the anionic denaturant comprises SDS, sodium deoxycholate, sodium cholate, sodium lauroyl sarcosinate, sodium caprylate, sodium N-lauroylsarcosine, lithium dodecyl sulfate, or sodium N-undecylenoyl sarcosinate. In some embodiments, the neutral denaturant comprises urea. In some embodiments, the cationic denaturant comprises guanidine hydrochloride. In some embodiments, the urea is at a final concentration of about 0.5 M to about 2 M. In some embodiments, the urea is at a final concentration of about 1 M. In some embodiments, the guanidine hydrochloride is at a final concentration of about 0.5 M to about 2 M. In some embodiments, the guanidine hydrochloride is at a final concentration of about 1 M. In some embodiments, the SDS is at a final concentration of about 0.1% (w/v) to about 2% (w/v). In some embodiments, the SDS is at a final concentration of about 0.1% (w/v) to about 0.5% (w/v). In some embodiments, the SDS is at a final concentration of about 0.5% (w/v). In some embodiments, the SDS is at a final concentration of about 0.25% (w/v). In some embodiments, the quenching buffer further comprises pullulan. In some embodiments, the pullulan promotes nucleic acid hybridization. In some embodiments, the pullulan is at a final concentration of about 0.1% (w/v) to about 1% (w/v). In some embodiments, the pullulan is at a final concentration of about 0.5% (w/v). In some embodiments, the quenching buffer further comprises pullulan. In some embodiments, the pullulan is at a final concentration of about 0.1% (w/v) to about 1% (w/v). In some embodiments, the pullulan is at a final concentration of about 0.5% (w/v).

In some embodiments, the test sample is a sputum sample.

In some embodiments, the method detects airway eosinophilia in a subject. In some embodiments, the method further comprises a method of diagnosing airway eosinophilia in a subject. In some embodiments, the method further comprises guiding treatment of airway eosinophilia in the subject.

Also provided is a method of detecting the presence or absence of eosinophil peroxidase in a test sample from a subject, comprising:
applying i) a mixture comprising the test sample, the catalytic nucleic acid described herein which is immobilized to a solid support, and a running buffer, or ii) a mixture comprising the test sample, a bridging region released from an immobilized activated catalytic nucleic acid, and a running buffer, to the sample pad of the lateral flow device described herein,
wherein the catalytic nucleic acid comprises a substrate comprising a ribonucleotide cleavage site,
wherein a quenching buffer is added to the running buffer to quench activity of the catalytic nucleic acid prior to applying the mixture to the sample pad,
a) wherein if the test sample comprises eosinophil peroxidase,
   i) contacting the eosinophil peroxidase with the immobilized catalytic nucleic acid, and activating the catalytic nucleic acid which cleaves the substrate at the ribonucleotide cleavage site and releases a bridging region comprising the reagent zone test oligonucleotide binding domain and the sensor zone test oligonucleotide binding domain,
   ii) allowing the running buffer and the released bridging region to laterally flow into the sensor zone, wherein a complex is formed between the bridging region and the reagent zone test oligonucleotide,
   iii) allowing the running buffer, the complex, and the reagent zone control oligonucleotide to laterally flow to the sensor zone,
   iv) allowing the complex to be captured by the sensor zone test oligonucleotide to produce a signal at a test line,
   v) detecting the signal in iv) in the sensor zone at the test line, optionally the signal is a color change signal,
   vi) allowing the reagent zone control oligonucleotide to be captured by the sensor zone control oligonucleotide to produce a signal at a control line, and
   vii) detecting the signal in vi) in the sensor zone at a control line, optionally the signal is a color change signal,
   whereby the detection of signals in both v) and vii) is indicative of the presence of eosinophil peroxidase and the correct functioning of the lateral flow device;
   or
b) wherein if the test sample does not comprise eosinophil peroxidase,
   the immobilized catalytic nucleic acid is not activated and no bridging region is released,
   i) allowing the running buffer to laterally flow into the reagent zone, and then the running buffer and the reagent zone control oligonucleotide to laterally flow to the sensor zone,
   ii) detecting no signal in the sensor zone at the test line,
   iii) allowing the reagent zone control oligonucleotide to be captured by the sensor zone control oligonucleotide to produce a signal, and
   iv) detecting the signal in the sensor zone at a control line, optionally the signal is a color change signal,
   whereby the detection of no signal in ii) and the detection of the signal in iv) are indicative of the absence of eosinophil peroxidase and the correct functioning of the lateral flow device.

In some embodiments, the method comprises incubating the mixture comprising the test sample, the catalytic nucleic acid described herein which is immobilized to a solid support, and the running buffer, prior to applying the mixture or a portion thereof to the sample pad. In some embodiments, the test sample comprises a clinical sample. In some embodiments, the test sample comprises a sputum sample. In some embodiments, the running buffer comprises from about 1 mM to about 3 mM dithiothreitol (DTT). In some embodiments, the running buffer comprises about 2 mM DTT. In some embodiments, DTT is for use to effectuate the breakdown of sputum in the sputum sample. In some embodiments, catalytic activity of the catalytic nucleic acid is arrested by the quenching buffer. In some embodiments, the quenching buffer comprises a denaturant. In some embodiments, the denaturant is an anionic denaturant, a neutral denaturant, or a cationic denaturant. In some embodiments, the denaturant is for binding to protein. In some embodiments, the quenching buffer comprises an anionic denaturant. In some embodiments, the anionic denaturant comprises SDS, sodium deoxycholate, sodium cholate, sodium lauroyl sarcosinate, sodium caprylate, sodium N-lauroylsarcosine, lithium dodecyl sulfate, or sodium N-undecylenoyl sarcosinate. In some embodiments, the neutral denaturant comprises urea. In some embodiments, the cationic denaturant comprises guanidine hydrochloride. In some embodiments, the urea is at a final concentration of about 0.5 M to about 2 M. In some embodiments, the urea is at a final concentration of about 1 M. In some embodiments, the guanidine hydrochloride is at a final concentration of about 0.5 M to about 2 M. In some embodiments, the guanidine hydrochloride is at a final concentration of about 1 M. In some embodiments, the SDS is at a final concentration of about 0.1% (w/v) to about 2% (w/v). In some embodiments, the SDS is at a final concentration of about 0.1% (w/v) to about 0.5% (w/v). In some embodiments, the SDS is at a final concentration of about 0.5% (w/v). In some embodiments, the SDS is at a final concentration of about 0.25% (w/v). In some embodiments, the quenching buffer further comprises pullulan. In some embodiments, the pullulan promotes nucleic acid hybridization. In some embodiments, the pullulan is at a final concentration of about 0.1% (w/v) to about 1% (w/v). In some embodiments, the pullulan is at a final concentration of about 0.5% (w/v). In some embodiments, the quenching buffer further comprises pullulan. In some embodiments, the pullulan is at a final concentration of about 0.1% (w/v) to about 1% (w/v). In some embodiments, the pullulan is at a final concentration of about 0.5% (w/v). In some embodiments, the method detects airway eosinophilia in the subject. In some embodiments, the method further comprises a method of diagnosing airway eosinophilia in the subject. In some embodiments, the method further comprises treating airway eosinophilia in the subject.

Also provided is a kit for detecting airway eosinophilia in a test sample, wherein the kit comprises a catalytic nucleic acid probe described herein, a biosensor described herein or components required for a method described herein, and instructions for use of the kit. In some embodiments, the kit further comprises reagents and/or solutions, such as buffers, to provide conditions for cleavage by the catalytic nucleic acid described herein. In some embodiments, the buffers comprise a running buffer, optionally HEPES buffer. In some embodiments, the buffers comprise a quenching buffer. In some embodiments, the quenching buffer comprises a denaturant. In some embodiments, the denaturant is for binding to protein. In some embodiments, the denaturant is an anionic denaturant, a cationic denaturant, or a neutral denaturant. In some embodiments, the anionic denaturant comprises, SDS, sodium deoxycholate, sodium cholate, sodium lauroyl sarcosinate, sodium caprylate, sodium N-lauroylsarcosine, lithium dodecyl sulfate, or sodium N-undecylenoyl sarcosinate. In some embodiments, the cationic denaturant comprises guanidine hydrochloride. In some embodiments, the neutral denaturant comprises urea. In some embodiments, the quenching buffer comprises urea. In some embodiments, the quenching buffer comprises guanidine hydrocholoride. In some embodiments, the quenching buffer comprises SDS. In some embodiments, the quenching buffer further comprises a pullulan. In some embodiments, the kit further comprises a test sample collection device. In some embodiments, the sample collection device is a swab or a container. In some embodiments, the kit comprises a label for identifying the test sample. In some embodiments, the kit comprises a package for the kit.

Also provided herein is use of the catalytic nucleic acid probe, the biosensor or the kit disclosed herein, to determine the presence or absence of eosinophil peroxidase.

### EXAMPLES

The following non-limiting Examples are illustrative of the present disclosure:

### Example 1: In Vitro Selection of EPX DNAzyme and Sequence Optimization

### Materials and Methods

DNA oligonucleotides. Nucleotides having nucleic acid sequences used in this disclosure are listed in Table 1, Table 2, and Table 4. The random DNA library (DL) and the fluorogenic substrate (FS) were purchased from Keck Oligo Synthesis Facilities, Yale University (New Haven, CT). All other sequences including the PCR primers (FP, RP1, RP2), the ligation template (LT) and the DNAzyme sequences obtained after selection were purchased from Integrated DNA technologies (IDT; Coralville, IA). DL contains 80 nucleotides (nt) including 50 random nucleotides denoted by N50 in the center and two constant regions of 16 nt and 14 nt at the 5′ and 3′ ends, respectively. Each random position, N, represents a 25% probability of A, C, G or T nucleotides. The 30-nt FS contains a riboadenosine nucleotide (R) that serves as the cleavage site. R is flanked by a fluorescein-dT (F) and a dabcyl-dT (Q). RP1 and RP2 are two reverse primers used in PCR. RP2 contains a poly-A tail (A20) at the 5′ separated by a triethylene glycol spacer (L, spacer 9 of IDT). The spacer prevents the poly-A tail from being amplified and thus produces a non-sense strand of the PCR product that is 20 nucleotides longer than the sense strand (DNAzyme), which allows purification of the desired sense sequence by denaturing polyacrylamide gel electrophoresis (dPAGE) (further details are provided below in the in vitro selection procedure). All the sequences were purified by 10% denaturing polyacrylamide gel electrophoresis (dPAGE) before use.

Enzymes and chemicals. T4 polynucleotide kinase (PNK) and T4 DNA ligase, including their respective buffers, were purchased from Thermo Scientific (Ottawa, ON, Canada). Biotools Thermophylus DNA polymerase was supplied by Mandel Scientific (Guelph, ON, Canada). Full length EPX was purchased from four different suppliers: Lee Biosciences (Cat. No. EPX-342-60-0.1, Fitzgerald (Cat. No. 118-30-1101), Sigma (Cat. No. SRP6158-10UG) and LSBIO (Cat. No. LS-G31098). EPX from each supplier was tested for peroxidase activity using a standard TMB-based peroxidase assay. Briefly, all EPX proteins from different vendors were prepared as 1 μM stock solutions in ddH₂O. 50 μL of the ready to use TMB solution (Neogen Corporation, cat #308175, KY, USA) was dispensed in microcentrifuge tubes labelled for each vendor. Next, 1 μL of each EPX was added to the designated tubes for each supplier. The reaction was conducted at room temperature (RT) for 10 min and the color appearance was monitored by eye. EPX from Lee Biosciences and Fitzgerald showed high and approximately equal activity, while EPX from both Sigma and LSBIO was inactive. Therefore, EPX from Lee Biosciences was employed in the selection. LPO (Lactoperoxidase, cat #LS000151), MPO (Myeloperoxidase, manufactured by Lee Biosciences and supplied by Cedarlane, Burlington, cat #MPO-426-10), TPO (Thyroperoxidase cat ##ABIN934717) were purchased from Cedarlane (Burlington, Canada). Eosinophil cationic protein (ECP, cat #E3-5319H), Eosinophil Derived Neurotoxin (EDN, cat #E2-779H) were purchased from Creative enzymes. Proteoglycan 2 pro eosinophil major basic protein (PGR2, cat #PRG2-44H) was obtained from Cedarlane. Ribonuclease H (RNAse H, cat #M0297S) and RNase A (cat #T3018L) were obtained from New England Biolabs. DNase I was obtained from Thermo Scientific (cat #EN0521). Unless otherwise noted, all other chemicals were purchased either from Bioshop Canada (Burlington, ON, Canada) or from Millipore-Sigma (Oakville, ON, Canada) and used without further purification. Water used herein was double-deionized (ddH₂O) and further purified using a Millipore Advantage A10 system (Millipore-Sigma) and autoclaved.

### In Vitro Selection

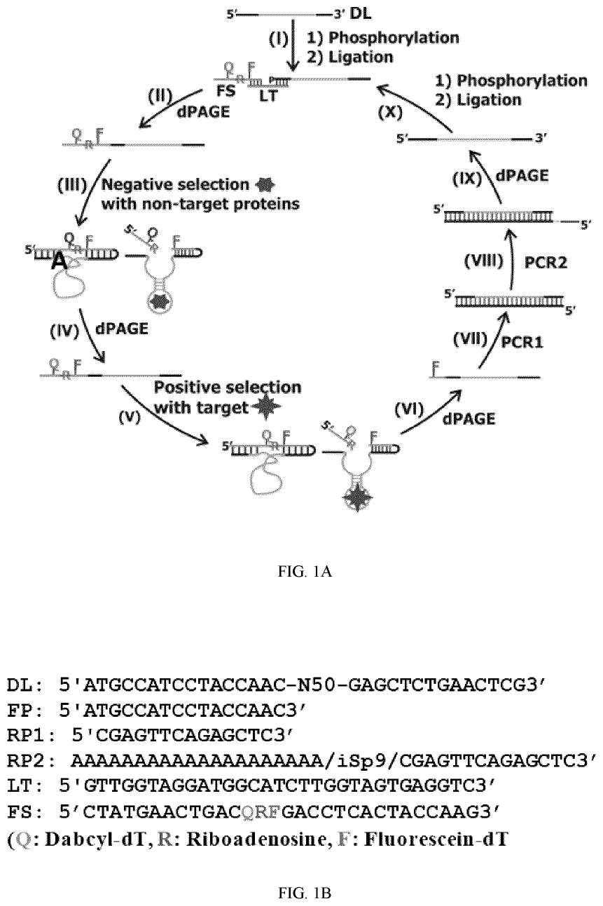

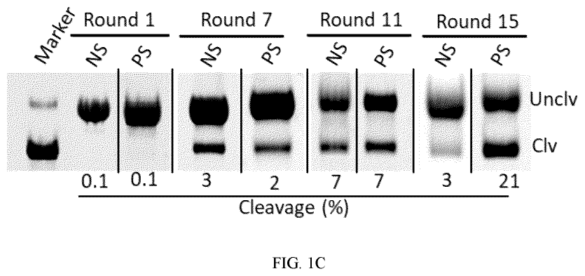

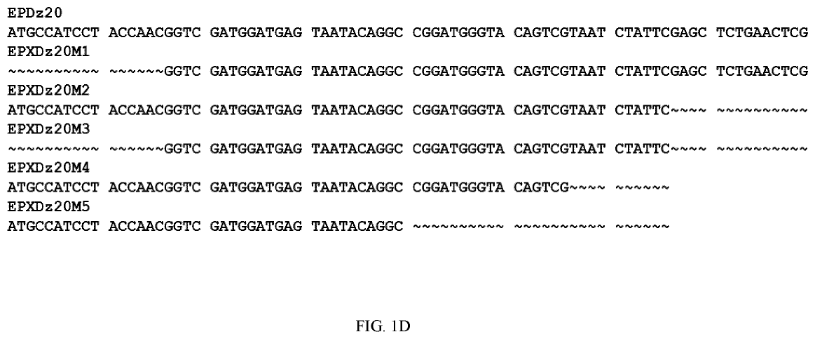

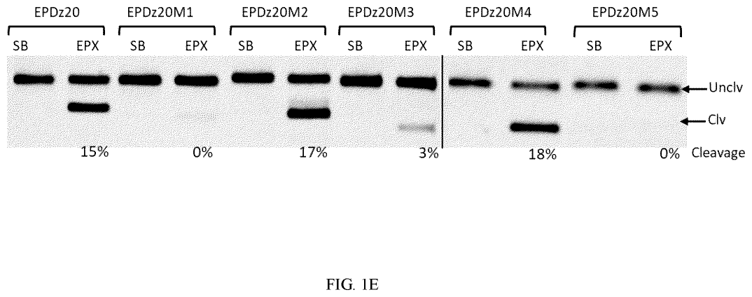

Schematic illustration of in vitro selection is shown in FIG. 1A.

Library preparation and selection (step I). In vitro selection was carried out with the DNA library (DL) covalently linked to the substrate FS. 1.0 nmole of DL was enzymatically ligated to FS as follows: the 5′-hydroxyl group of DL was phosphorylated using 15 units of T4 PNK for 45 min at 37° C. in 1×T4 polynucleotide kinase buffer A (PKB) in the presence of 1 mM ATP in a 100 μL reaction volume. The reaction was stopped by heating at 90° C. for 5 min. Equivalent amounts of FS and ligation template (1.0 nmole each) were added to this solution and, after brief vortexing, the mixture was heated at 90° C. for 1 min and cooled to room temperature (RT) for 20 min. Then, 30 μL of 10× T4 DNA ligase buffer (T4LB), 30 μL of PEG4000 and 5 μL (25 U) of T4 DNA ligase (T4DL) were added. The volume was adjusted to 300 μL with ddH₂O. After pipette mixing, the reaction mixture was incubated at room temperature (RT) for 1 h.

Purification of ligated FS-DL (step II). The DNA molecules in the reaction mixture from step I were isolated by ethanol precipitation and the ligated FS-DL molecules were purified by 10% dPAGE and ethanol precipitation. The isolated DNA was dried by heating the tube at 95° C. keeping the lid open for 3 min.

Counters election (step III). For the counter selection, a pool of non-target proteins consisting of LPO, MPO and TPO at 1 μM concentration each was prepared in 1× selection buffer (1×SB) (50 mM HEPES, pH 7.5, 150 mM NaCl, 15 mM MgCl₂, and 0.01% Tween20). The DNA pool obtained in step II was dissolved in 200 μL of 1×SB. 25 μL of a mixture of non-target proteins was mixed with the FS-DL pool. The volume was adjusted to 250 μL with 1×SB. After pipette mixing, the reaction mixture was incubated at room temperature for 2 h. The reaction was quenched by the addition 25 μL of 3.0 M NaOAc (pH 5.2) and 675 μL of 100% cold ethanol. The mixture was left at −20° C. for 2 hours to precipitate the DNA molecules. The DNA molecules were isolated by centrifugation for 25 min at 14000 rpm at 4° C. The supernatant was discarded, and the DNA palette was dried at 70° C. for 5 min to evaporate the ethanol.

Purification of uncleaved FS-DL (step IV). The above DNA pellet, after ethanol precipitation, was subjected to 10% dPAGE and the uncleaved FS-DL molecules were isolated as described in step II.

Positive selection (step V). The purified uncleaved full length DNA pool, obtained from step IV, was dissolved in 200 μL of 1×SB. A stock of 1 μM EPX in 1×SB was prepared and 25 μL of this EPX was added to the DNA pool. The volume was adjusted to 250 μL by adding 25 μL 1×SB. After pipette mixing, the reaction mixture was incubated at room temperature for 60 min. The reaction was stopped by adding 25 μL of NaOAc (pH 5.2) followed by 675 μL of 100% cold ethanol.

Isolation of cleaved products (step VI). After ethanol precipitation, the reaction mixture was subjected to 10% dPAGE. Before loading the sample in the gel, a marker was prepared by treating a small portion of the FS-DL with 0.25 M NaOH at 90° C. for 10 min. In the first few rounds of selection, the cleaved product band in the gel image is not expected to be visible owing to a low amount of cleaved DNA. Therefore, based on the position of the marker band, a portion of the gel below the uncleaved full length DNA band was excised and the DNA molecules were isolated. After ethanol precipitation the DNA molecules were dissolved in 50 μL ddH₂O.

PCR1 (step VII). PCR was typically conducted in a volume of 50 μL with 10 μL of the isolated DNA molecules in step VI in presence of 0.5 μM each of FP and RP1, 200 μM dNTPs, 1×PCR buffer (75 mM Tris-HCl, pH 9.0, 2 mM MgCl₂, 50 mM KCl, 20 mM (NH₄)₂SO₄) and 2.5 units of Thermus thermophilus DNA polymerase (Biotools, through Mandel Scientific; Guelph, ON, Canada). The amplification was conducted using the following thermocycling program: one cycle of 94° C. for 1 min; 13 cycles of 94° C. for 30 s, 50° C. for 45 s and 72° C. for 45 s (the numbers of amplification cycles between different selection rounds were adjusted, typically between 13 and 15 cycles, to achieve full amplification (analyzed by 2% agarose gel electrophoresis); one cycle of 72° C. for 1 min.

PCR2 (step VIII). Because of the requirement for a large amount of DNA molecules in the selection, a second PCR step was conducted in 20 tubes of 50 μL each using the PCR1 product as a template. In this case, 1 μL of the PCR1 product was diluted to 20 μL with ddH₂O, 1 μL of this diluted PCR1 was used in PCR2 using FP and RP2 primers following the same amplification program as was used for PCR1.

Purification of DNAzyme sense-strand (step IX). The PCR product from the above step was concentrated by ethanol precipitation and subjected to 10% dPAGE. The DNA band of the sense-strand (shorter sequence, bottom band) was excised, and the DNA was eluted and stored at −20° C. as a dry pellet until use.

Ligation of PCR product to FS (step X) and repetition of steps II-X. The purified DNA (approximately 200 pmole) in the above step was ligated to FS as follows: The PCR product was phosphorylated in a 100 μL reaction volume with 10 U of PNK in the presence of 1 mM ATP in 1×PNK buffer for 40 min at 37° C. The reaction volume for phosphorylation of the PCR product for the subsequent rounds was constantly maintained at 100 μL. The kinase reaction was quenched by heating at 90° C. for 5 min and cooled down to RT for 20 min. Equal amounts of FS and LT (200 pmole each) were added to the PNK reaction mixture, mixed by vortexing, heated at 90° C. for 1 min and cooled to RT for 20 min. Then, 20 μL of T4LB, 20 μL of PEG4000 and 4 μL of T4DL were added sequentially and the volume was adjusted to 200 μL with ddH₂O (ligations for all the subsequent selection rounds were carried out in a 200 μL volume). After pipette mixing, the ligation reaction was conducted at RT for 1 h. After ethanol precipitation, the ligated DNA product was purified by 10% dPAGE and employed in the second round of negative and positive selections following the same procedure as described in the above steps for the first round. A significant amount of cleavage product was obtained at round 15 of selection (FIG. 1C) and finally the DNA population of this round was amplified by PCR using specific primers for deep sequencing (Illumina deep sequencing at Farncombe Metagenomics Facility, McMaster University central facility). The sequences were obtained in FASTQ file format and analyzed using Oracle VirtualBox sequence analyzer. Note that one round of counter selection was applied for every two rounds of positive selection to achieve selectivity.

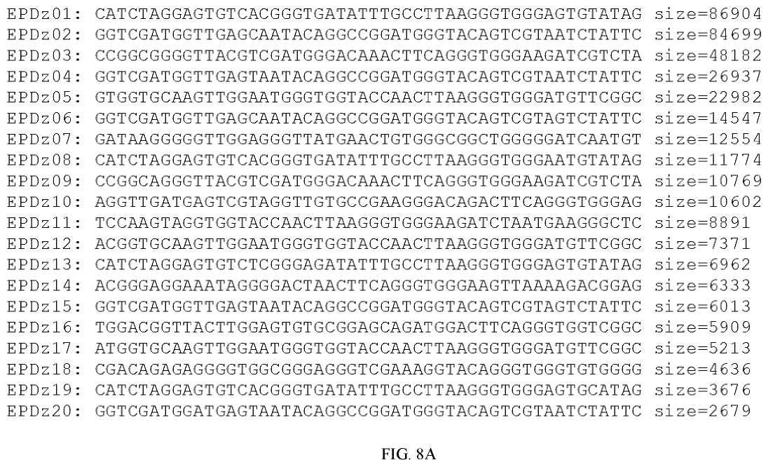

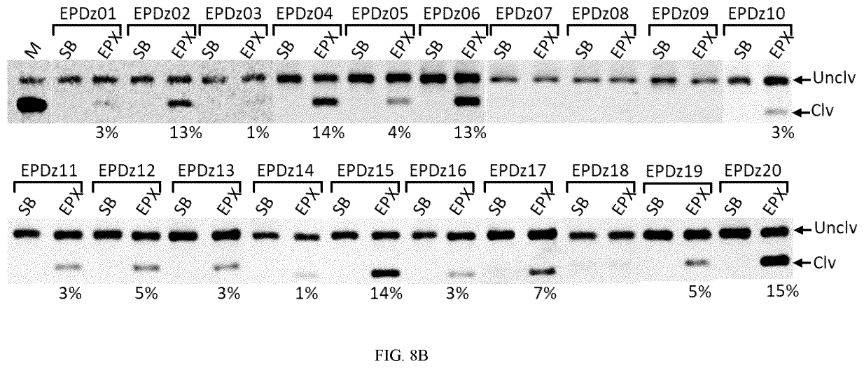

Cleavage tests of top 20 sequences. The top 20 DNAzyme sequences based on the frequency (or abundance) including their names and the read numbers are shown in FIG. 8A and Table 2. Each of these 20 sequences were synthesized and tested for their cleavage performance. Each DNAzyme (1.0 nmole) was individually ligated to FS as described above in the selection procedure. After ligation and purification, the DNAzyme sequences were dissolved in ddH₂O and quantified using a Tecan NanoQuant™ plate as per the instruments user's guide. The concentration of each DNAzyme was adjusted to 2.5 μM with ddH₂O and stored at −20° C. until use. Each of the DNAzyme cleavage reactions was conducted in a 10 μL volume. Specifically, 1 μL of each DNAzyme was transferred to a microcentrifuge tube designated for each DNAzyme sequence followed by addition of 5 μL of 2×SB. The cleavage reaction was started by adding 4 μL of ddH₂O in tube 1 (control), and 4 μL of EPX (100 nM in 1×SB). After 45 min of incubation at RT the cleavage reactions were quenched by adding 10 μL of 2× gel loading buffer (GLB). The reaction mixtures were analyzed by 10% dPAGE and the gel was scanned for fluorescence bands using a Chemidoc™ fluorescent imager (Bio-Rad, Hercules, CA).

To calculate percent cleavage, the equation accounts for the FS substrate when fully cleaved will produce a 6-fold higher fluorescence compared to the uncleaved substrate. Therefore, cleavage percentage was calculated using the following equation:

$$\% Cleavage = 100 \times [ ( {F}_{clv} / 6 ) / \{ ( {F}_{clv} / 6 ) + {F}_{unclv} \} ] \qquad [ Eq . 1 ]$$

where, F_{clv} denotes fluorescence intensity of the gel band for the cleaved DNAzyme product and F_{unclv} denotes fluorescence of gel band for the uncleaved DNAzyme. This equation produced almost identical cleavage percentage from fluorescence data as is obtained using phosphorimaging of radiolabelled substrates.

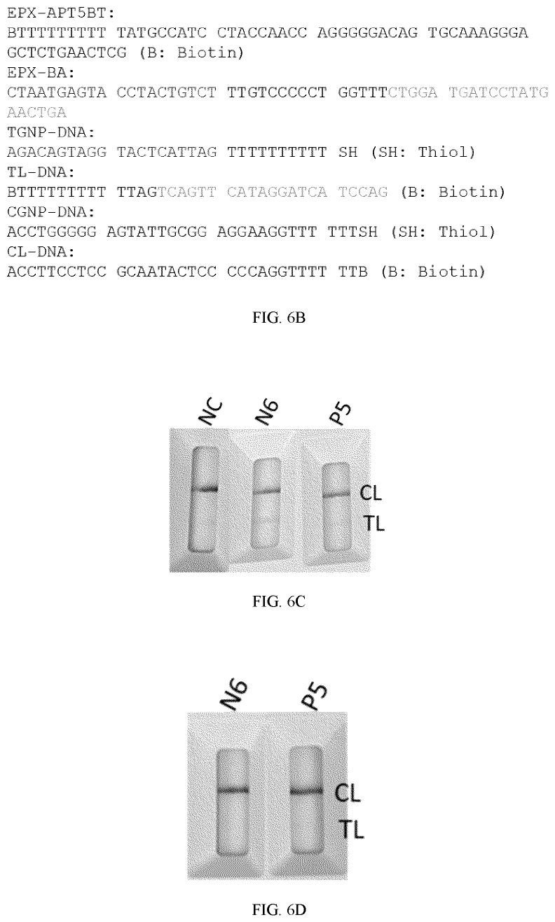

Sequence truncation and cleavage test. Based on the cleavage performance of the top 20 DNAzymes (FIG. 6B) EPDz20 was selected for nucleotide truncation to shorten the DNAzyme for subsequent experiments. For this purpose, the primers were deleted one at a time and together, and further truncations were then performed from either the 3′ or 5′ terminus (see FIG. 1D). To test activity, 1000 pmole of the truncated sequences (M1-M5) were ligated to the substrate FS as described above and purified by 10% dPAGE. The cleavage performance of each truncated sequence was assessed by 10% dPAGE in the same way as described above (FIG. 1E). A shortened sequence with full activity named EPDz20M4 was identified and was used in all subsequent experiments of the disclosure.

### Results and Discussion

FIG. 1A shows the in vitro selection method employed to isolate RCDs that were selectively activated by EPX. A random DNA library (DL, ˜10¹⁴ sequences) was first ligated to a fluorogenic substrate (FS) containing a single ribonucleotide flanked by a fluorophore and quencher (see Table 1 and FIG. 1B for relevant sequences). Negative selection was first done with a mixture of lactoperoxidase (LPO), thyroperoxidase (TPO) and MPO, which are commonly found in sputum. DNAzymes that did not cleave were isolated by denaturing polyacrylamide gel electrophoresis (dPAGE) and carried forward to the first round of positive selection with EPX to allow EPX-dependent cleavage of the fluorogenic DNA substrate. The cleaved DNA molecules were isolated by dPAGE, followed by two rounds of PCR amplification using FP, RP1 and RP2 as forward and reverse primers. The amplified PCR products were ligated to FS using a ligation template (LT), and negative and positive selection steps were continued until a significant amount of cleaved product was obtained in the positive selection round relative to the previous round of negative selection.

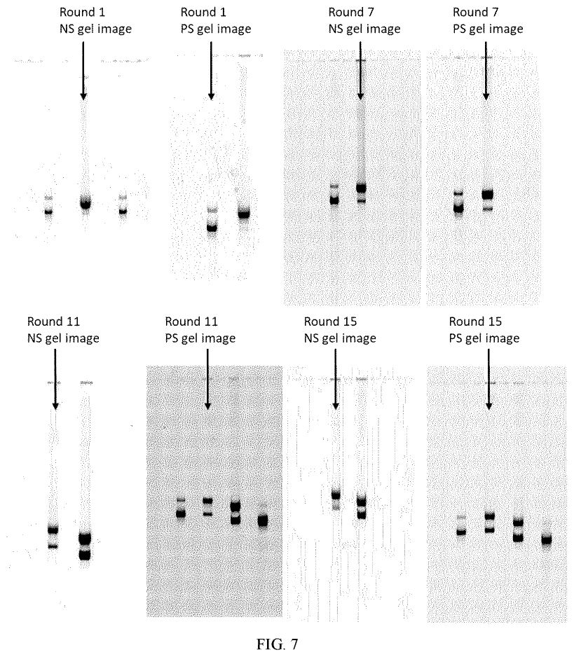

As shown in FIG. 1C (full gel shown in FIG. 7), similar levels of cleavage product were obtained in the positive and negative selection pools after 7 rounds of selection. However, the amount of cleavage product obtained from counter selection was greater than for the positive selection. By round 11, the cleavage product band was more intense for the positive selection than the negative selection, while round 15 produced 7-fold greater cleavage product for positive selection relative to negative selection (FIG. 1C), at which point selection was terminated.

The population from round 15 was used for deep sequencing and the top twenty sequences (FIG. 8A) were synthesized and tested for cleavage performance. The results showed that several of the RCDs cleaved the ligated FS in presence of EPX but not in the presence of selection buffer (SB) (FIG. 8B), with the RCD denoted as EPDz20 showing the greatest degree of cleavage with EPX relative to SB (15% cleavage in 30 min). These results indicate that EPDz20 can be allosterically activated upon binding to the EPX protein.

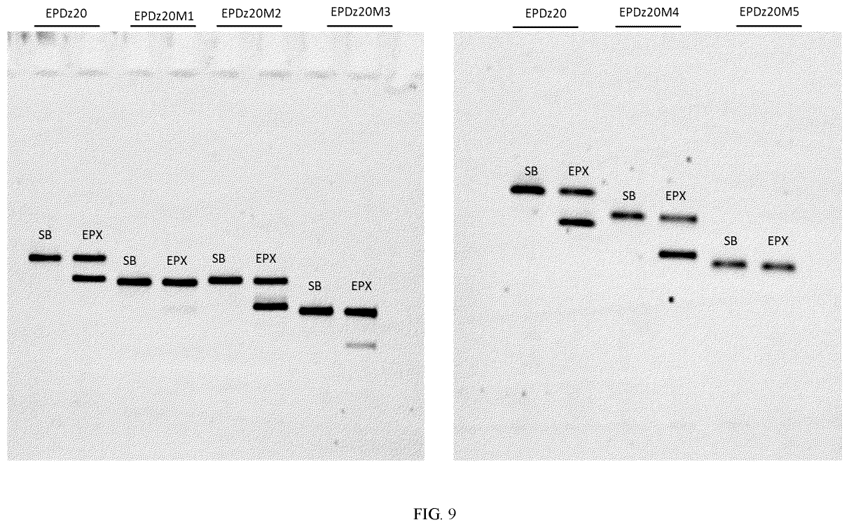

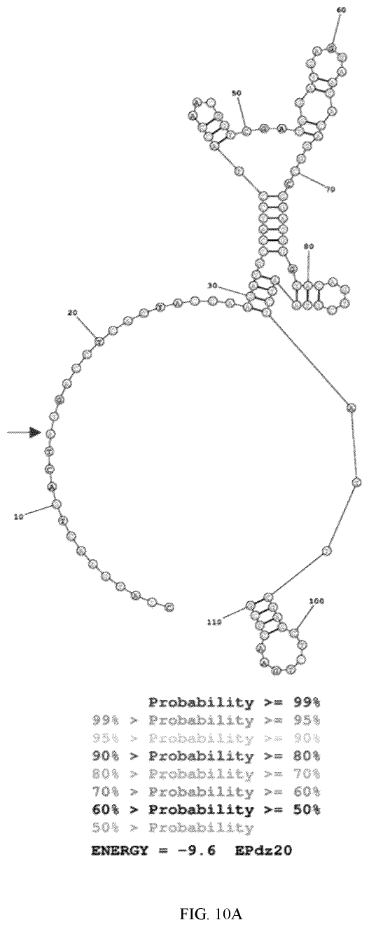

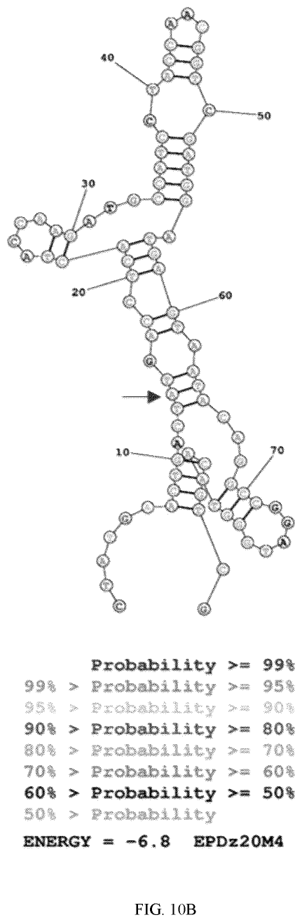

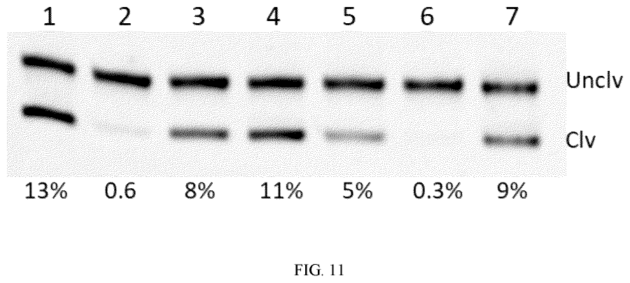

Truncation analysis was carried out to shorten the EPDz20 DNAzyme (FIG. 1D). One of the truncated versions, a 56 nt DNAzyme denoted as EPXDz20M4, generated 18% cleavage in 30 min (FIG. 1E, full gel shown in FIG. 9), and was used in all subsequent experiments. The predicted secondary structures of EPDz20 and EPDz20M4, with FS ligated, are provided in FIG. 10A and FIG. 10B. Evaluation of different buffer conditions indicated that selection buffer (50 mM HEPES, 150 mM NaCl, 15 mM MgCl₂, 0.01% Tween 20, pH 7.5) produced the highest cleavage activity (FIG. 11), and hence the original selection buffer was used in all further experiments herein unless otherwise noted.

### Example 2: Evaluation of EPXDz20M4 Selectivity and Sensitivity

### Materials and Methods

Selectivity tests by PAGE in buffer. Selectivity was tested against a series of proteins, including MPO, TPO, LPO, ECP and EDN, which are closely related to EPX or found at high concentrations in eosinophils, PRG 2 (proteoglycan 2, pro eosinophil major basic protein), which is a basic protein (pI>11) that was evaluated for potential non-specific binding, and cleavage of the DNAzyme. DNase I, RNase A and RNase H were also evaluated for nuclease resistance. In each case, a stock solution of EPDz20M4 (100 nM in 1×SB) was prepared and 9 μL of this solution was dispensed in separate tubes. A volume of 1 μL of each protein stock solution (1 μM stock solution in 1×SB) was then added to designated tubes. A positive control (PC) containing an identical concentration of EPX, and a negative control (NC) with only 1×SB were also prepared. Cleavage reactions were conducted at room temperature (RT) for 30 min and were immediately quenched by adding 10 μL of gel loading buffer (GLB) followed by analysis by 10% dPAGE. Fluorescence of the gel bands was obtained using a Chemidoc™ fluorescence imager and the percent cleavage for each protein was determined as outlined above.

Sensitivity tests by dPAGE in buffer. The cleavage activity of EPDz20M4 was evaluated using solutions (10 μL) containing 90 nM of the DNAzyme and a concentration of EPX ranging from 0-200 nM prepared in 1×SB. Reactions were performed for 30 min after which the reactions were immediately quenched by adding 10 μL of GLB, followed by 10% dPAGE. Fluorescence from the gel bands was determined as described above and the percent cleavage was obtained as outlined earlier.

### Results and Discussion

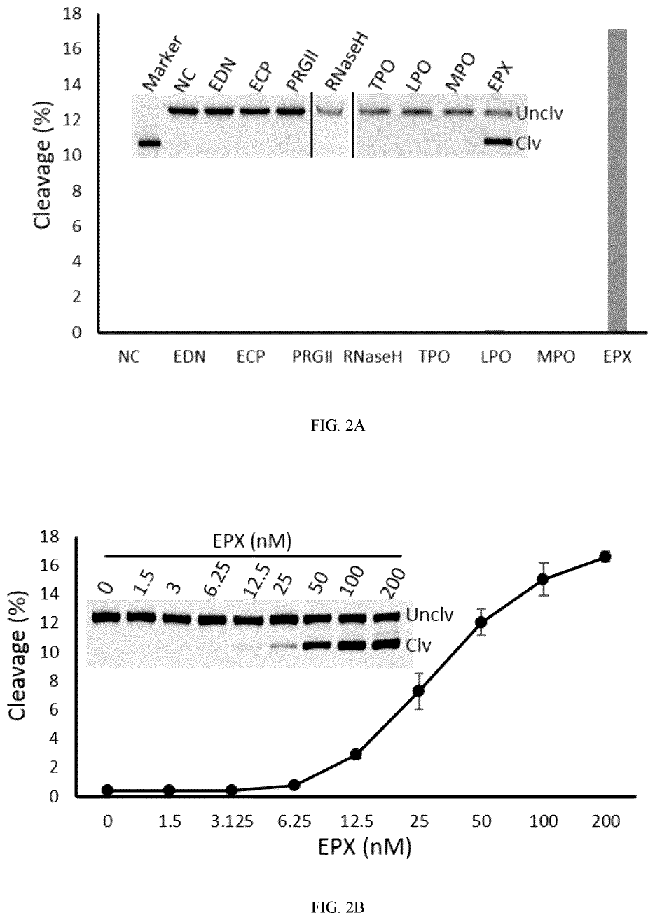

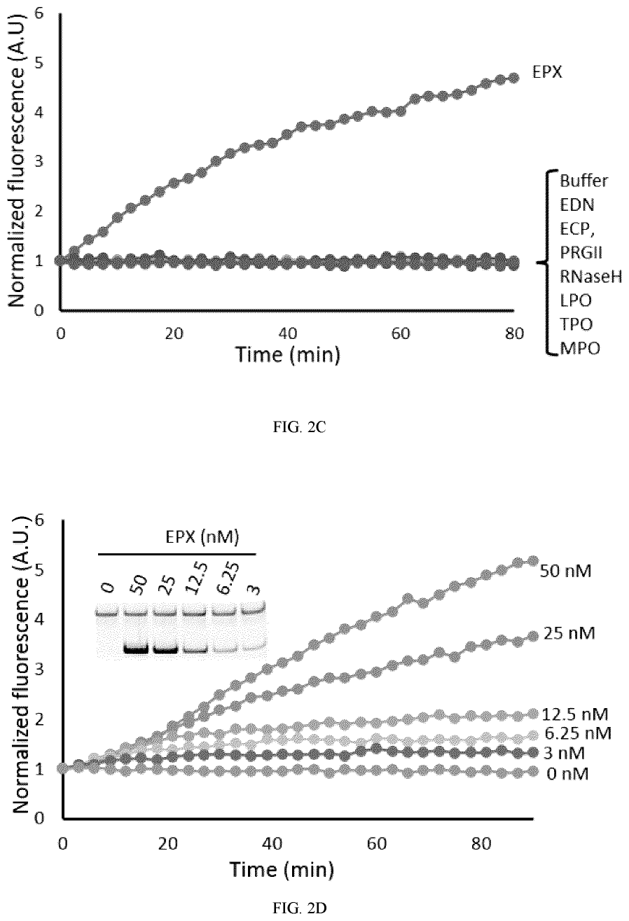

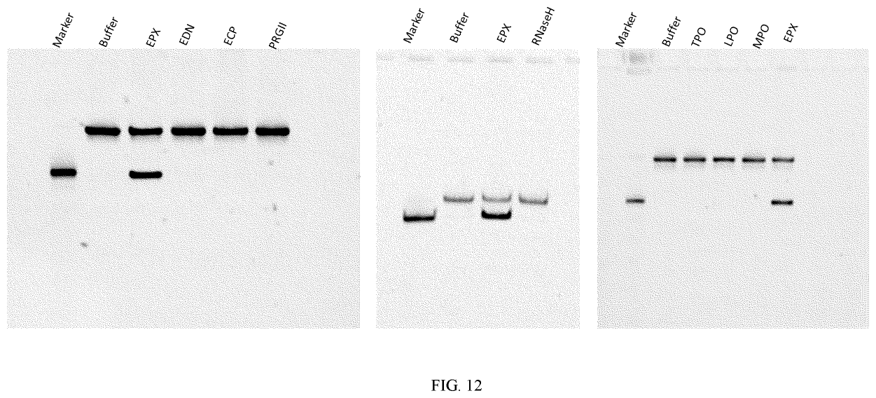

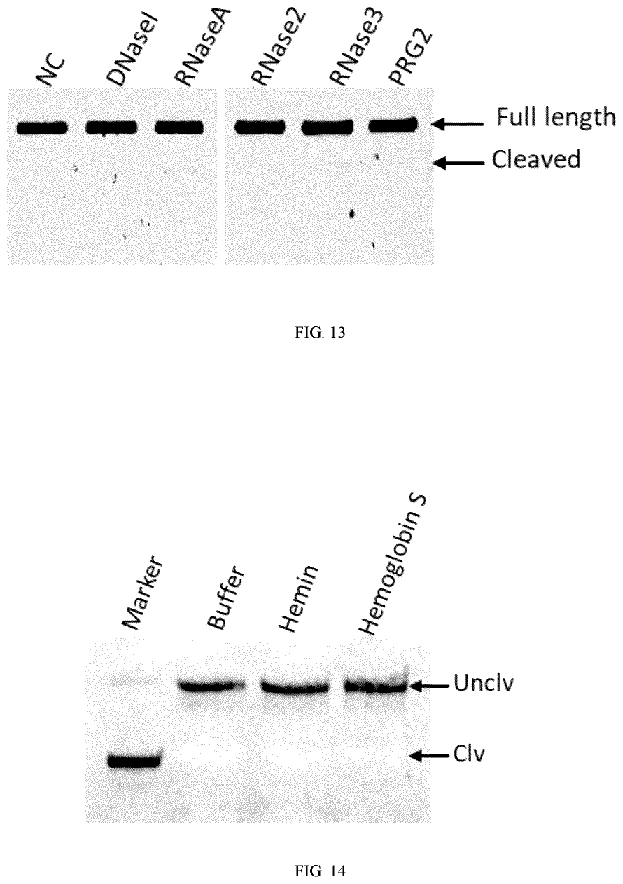

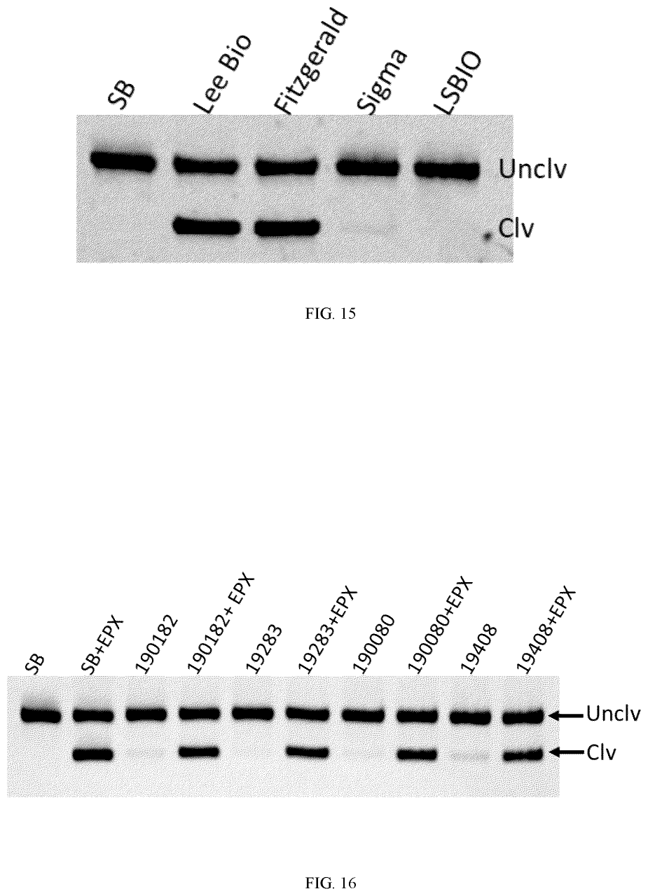

The selectivity and sensitivity of EPXDz20M4 were first tested in buffer by evaluating the extent of cleavage after 30 min via 10% dPAGE. The results shown in FIG. 2A (full gels shown in FIG. 12) revealed that EPDz20M4 produced cleavage products only when EPX was present, but not in the presence of TPO, LPO, or MPO), even though they are closely related to EPX. More importantly, the RCD was not activated by either the eosinophil cationic proteins EDN (RNAse 2) or ECP (RNAse 3), by PRGII (proteoglycan 2, pro eosinophil major basic protein) or by other ribonucleases (RNase A, RNAse H) or DNases (DNAse I), even though none of these proteins were present as counter selection targets (see FIG. 13). In addition, the DNAzyme was not cleaved by either hemin or hemoglobin S (FIG. 14), indicating that cleavage is not based solely on binding of the heme or Fe(II/III) ion in EPX. Taken together, these data show that the RCD has high selectivity for EPX as well as excellent resistance to mammalian nucleases and is not activated by highly basic proteins that might be expected to interact with the RCD electrostatically. Cleavage activity with different versions of EPX was also tested. These included two full length versions from two different suppliers that were isolated from human blood and showed high peroxidase activity, and full-length recombinant versions from two different suppliers that did not show any peroxidase activity (FIG. 15). Interestingly, the RCD was activated only in the presence of the active EPX, regardless of the supplier, suggesting that the active site must be intact to allow recognition by the RCD.

### Example 3: Fluorescence Signalling of EPXDz20M4

### Materials and Methods

Real-Time Fluorescence signaling. Selectivity and sensitivity of the EPDz20M4 DNAzyme was also assessed using real-time fluorescence signaling. To assess selectivity, solutions were prepared in microwell plates (black, clear flat bottom, Corning) consisting of 99 nM of EPDz20M4 and 50 nM of either EPX (positive control) MPO, TPO or LPO, EDN, ECP, PRG2 and RNaseH in a volume of 100 μL. A buffer blank with only the DNAzyme was also tested as a negative control. Immediately following addition of the relevant protein, fluorescence intensity was measured over a period of 90 min using a Tecan plate reader (M200) using excitation at 488 nm, emission at 520 nm, bottom read, and a gain of 150. Sensitivity was tested using a similar method except that in this case fluorescence was measured as a function of EPX concentration over the range of 0-50 nM.

### Results and Discussion

As shown in FIG. 2B, dPAGE analysis clearly indicates that the cleavage is also dependent on the concentration of EPX, producing an apparent dissociation constant (Kd) of ˜30 nM (EPX concentration producing 50% maximum cleavage) and an estimated detection limit of 6 nM with a 30 min cleavage reaction time. The maximum cleavage of ˜18% in 30 min with saturating amounts of EPX compares favourably to the cleavage rate and maximum cleavage obtained with bacteria-specific RCDs, which range from 14-39%, but is still slower than the best reported metal-ion dependent RCDs, which can show—0-90% cleavage in as little as 10 min and rate constants up to 10 min⁻¹. The relatively low cleavage is likely due to the presence of multiple conformations of the DNAzyme, including several inactive conformations.

The data above clearly demonstrate that it is possible to produce protein-selective RCDs using standard in vitro selection protocols which are similar to those previously used to generate bacteria-selective RCDs. The target of the present RCD, however, is well characterized, unlike the case for bacterial-specific RCDs, where the specific targets for these systems are not known.

Fluorescence monitoring was used to further evaluate the sensitivity and selectivity based on real-time signal generation. The results showed that the RCD produced an increase in fluorescence intensity only in the presence EPX (FIG. 2C), in agreement with the dPAGE data, and produced a detectable fluorescence signal with as low as 6 nM of EPX using a 45 min reaction time (FIG. 2D), confidence level of 95%, and an LOD of 25 nM using a 15 min reaction time (confidence level of 95%), which is sufficient to discriminate eosinophil positive and negative samples (see below). Analysis of reaction mixtures by dPAGE (inset to FIG. 2D) clearly indicated that the RCD cleaved the substrate at the expected cleavage junction. Based on these data, it should be possible to produce a fluorescence assay for EPX detection with an assay time of as little as 15 min, though a fluorescence reader, such as the portable ANDalyze reader, would be required in this case.

### Example 4: Development of Lateral Flow Devices

### Materials and Methods

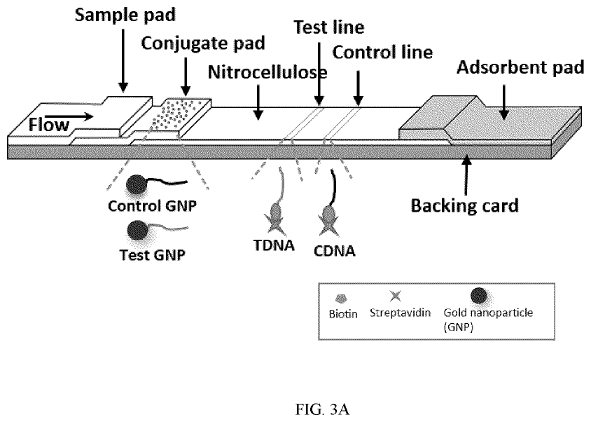

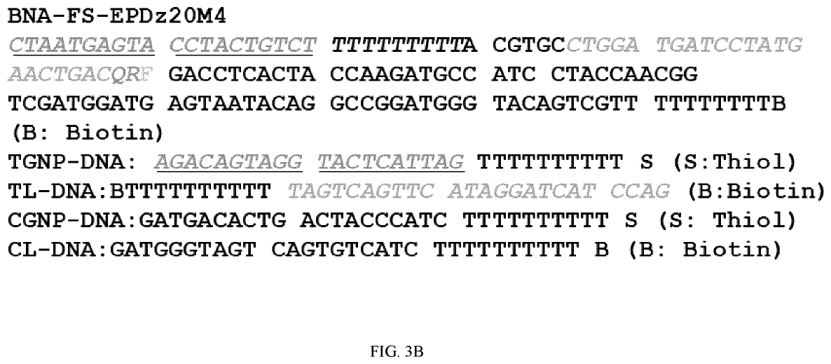

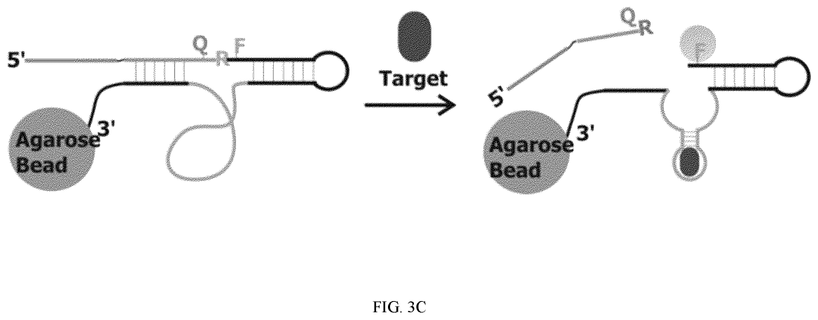

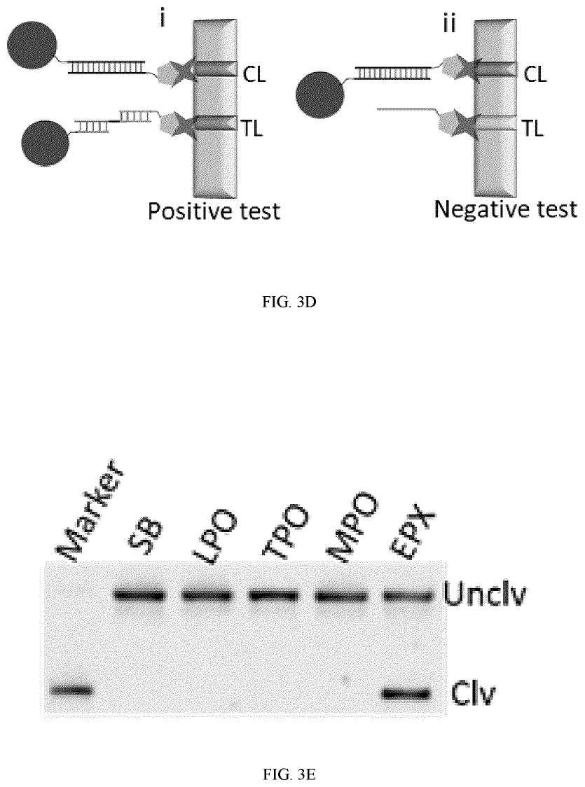

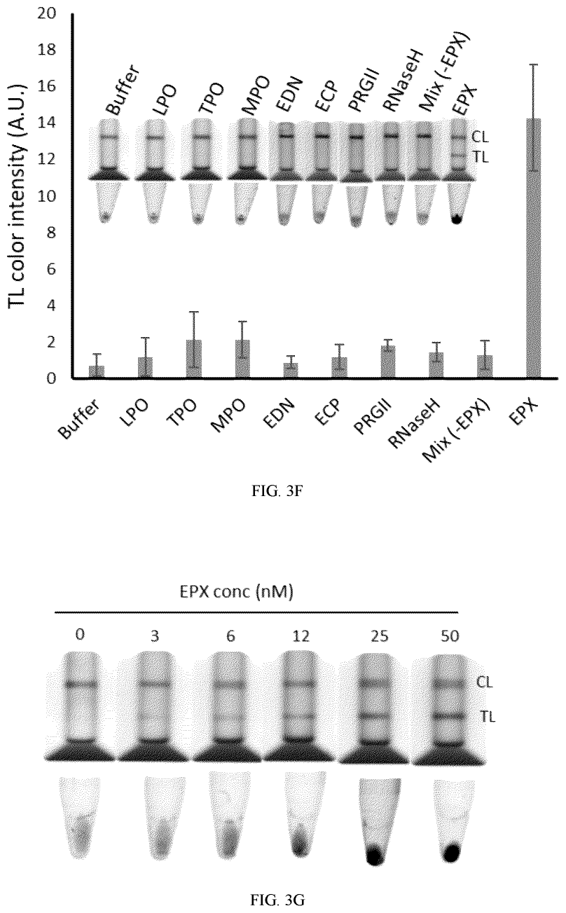

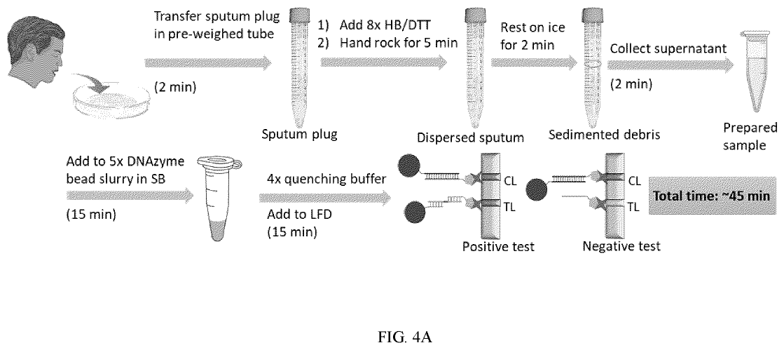

Coupling DNAs with GNPs. 40 nM citrate capped standard gold nanoparticles (GNPs) (Cyto Diagnostics, Burlington Canada) were used herein. TGNP-DNA and CGNP-DNA sequences (Table 1 and FIG. 3A), designed to be captured in the test and control line respectively, were individually coupled with the GNPs. In each case, 800 μL of GNP (OD 10) was added to 50 μL of TGNP-DNA or CGNP-DNA (250 μM stocks) and the tubes were rotated vertically at RT overnight. 50 μL of 1 M NaCl was then added to each tube, mixed, and rotated overnight again. A further 50 μL of 1 M NaCl was then added to each tube and these were again rotated at RT overnight. Next, 100 μL of 10% BSA was added to both tubes, mixed for 2 h at RT, followed by centrifuging at 5000 rpm for 15 min. The clear supernatant was discarded, and the GNP pellet was resuspended in 500 μL of ddH₂O, centrifuged again and the clear supernatant was discarded. Resuspension in ddH₂O and centrifugation was repeated one more time. Finally, the GNP pellet was resuspended in 800 μL of GNP suspension buffer (1×PBS including 0.1% Tween20 and 5% sucrose) and this stock of GNP conjugate was stored at 4° C. until use.

Printing and assembly of LFDs. Each LFD strip is composed of five components: 1) a backing card, 2) a printed nitro cellulose paper (NCP) membrane, 3) a sample pad, 4) a conjugate pad and 5) an adsorbent pad (see FIG. 3A for a schematic of the LFD). TL-DNA and CL-DNA (see sequences in Table 1 and FIG. 3B) were printed on the NCP membranes as follows: 120 μL of each DNA (250 μM) was mixed with 1 mL of streptavidin (0.5 mg/mL: ˜10 μM) in PBS (pH 7.4) and incubated at room temperature for 30 min. After incubation, the streptavidin-DNA conjugate was passed through a centrifugal column (Amicon® Ultra-0.5 mL, Millipore-Sigma) with a 50K molecular cut off size for 10 min at 14000×g. The conjugate was washed twice with 300 μL of PBS. After washing, the concentrated streptavidin-DNA was recovered by placing the filter device upside down into a fresh micro centrifuge tube followed by centrifugation at 21000×g for 2 min. The recovered streptavidin-DNA was diluted to a final volume of 800 μL in PBS. These streptavidin-DNA conjugates were printed on the nitrocellulose membrane (NCP: HF120plus thick, Cytiva, USA) using a Biodot liquid dispenser (XYZ3600, Irvine, CA, USA) with the following settings: Drop (nL): 15, rate (μL/cm): 0.375 μL, pitch: 40, speed: 40 mm/s, with a total of two print passes per printed line. Before printing, the NCP was cut into a 25×300 mm pieces and preconditioned at 60% relative humidity. Both the control and test lines (˜1 mm thick) were printed ˜20 mm (CL) and ˜15 mm (TL) below the top edge of the NCP membrane respectively with a 5 mm interline distance. After printing, the NCP was dried at 37° C. for 1 h. This printed NCP was attached to the middle of a backing card. The absorbent pad (Ahlstrom grade 270) was cut into the same length to fit the backing card (10×300 mm) size and attached to the backing card just above the control line of the strips with 1 mm overlap with the NCP membrane. The adsorbent pad and printed NCP attached backing card was cut into strips of 4 mm width using a Biodot Guillotine cutter.

A conjugate pad (standard17, Cytiva, USA) was cut into 4×20 mm sizes and the TGNP-DNA and CGNP-DNA conjugates prepared above were mixed in a 1:1 ratio. 10 μL of this GNP solution was dispensed on one end of the conjugate pad by pipette. After drying at 37° C. for 1 h, the GNP conjugate pad was attached just below the test line of the strip with a 1 mm overlap with the NCP membrane. The blank part of the conjugate pad at the bottom edge of the strip served as the sample pad. This assembled strip was placed in lateral flow cassette (Drummond scientific company, PA, USA) and used as the final LFD for testing samples.

Preparation of BNA-FS-EPDz20M4B. The modified DNAzyme, BNA-FS-EPDz20M4B, was prepared as follows: 1 nmole each of biotinylated EPDz20M and FS was phosphorylated separately in a 100 μL reaction volume in the presence of 10 units of PNK and 1 mM of ATP in 1×PNK buffer for 45 min at RT. The reaction was quenched by heating at 90° C. for 5 min. Next, 1 nmole of LT1 and LT2 (Table 1) was added to the reaction mixture and heated at 90° C. for 1 min. After cooling at RT for 20 min, 30 L of PEG4000, 30 μL of 10× T4 DNA ligase buffer and 5 μL of T4 DNA ligase were sequentially added to the reaction mixture. The volume was adjusted to 300 μL with ddH₂O and mixed by pipetting. The reaction was conducted at RT for 60 min. The ligated DNA molecules were isolated by ethanol precipitation and purified by 10% dPAGE. The purified BNA-FS-EPDz20M4B was dissolved in 100 μL ddH₂O, quantified using a Nanodrop™ plate and the concentration was adjusted to 2.5 μM with ddH₂O.

Cleavage test of BNA-FS-EPDz20M4B. To ensure retention of both the selectivity and sensitivity of the modified DNAzyme, cleavage reactions were carried out in 10 μL volume with EPX, MPO, TPO and LPO as described above for the unmodified DNAzyme, using 90 nM of the DNAzyme and 100 nM of each protein in 1×SB or only buffer as a blank. Cleavage reactions were conducted at room temperature (RT) for 30 min. The reactions were quenched by adding 10 μL of gel loading buffer (GLB) and subjected to 10% dPAGE and were quantified for percent cleavage as described above.

Immobilization of BNA-FS-EPDz20M4B on agarose beads. 100 μL of agarose bead suspension (Pierce™, Fisher Scientific Inc., Burlington, ON, Canada) was washed with 300 μL of 1×SB. The beads were resuspended in 300 μL of 1×SB and 50 pmole of the biotinylated DNAzyme (BNA-FS-EPDz20M4B) was added to the bead suspension and incubated at room temperature for 1 h in a vertical rotator to prevent the beads from settling. After incubation, the mixture was spun down by a benchtop centrifuge (2000×g, 30 s) and the clear supernatant was discarded. The bead complex was washed a total 5 times with 300 μL 1×SB to remove unbound DNAzymes and then resuspended in 500 μL for buffer (or 1000 μL for sputum samples) of 1×SB and stored at 4° C. until use.

### Results and Discussion

To produce an equipment-free rapid test for EPX, a recently reported DNAzyme-based lateral flow device for Staphylococcus aureus⁷ was adapted, where a modified version of the cleavage product acts as a bridging strand to allow capture of complimentary DNA (cDNA)-modified gold nanoparticles (GNPs) onto a DNA-modified test line. FIG. 3A shows a schematic illustration of the lateral flow device with all the components labelled, and FIG. 3B shows the sequences of all oligonucleotides used to produce the LFD. The fluorophore (F) and quencher (Q) labels were retained, as removal of these labels can render DNAzymes inactive. The bridging DNA was designed by adding an extended sequence domain to the 5′-end of the fluorescently labelled substrate within the DNAzyme (italicized, and italicized and underlined domains in BNA-FS-EPDz20M4 in FIG. 3B), while a biotin was added to the 3′ end to allow immobilization of the RCD onto streptavidin coated agarose beads. A thiolated cDNA, including a poly-T spacer, which was complementary to the 5′ end of the liberated cleavage product (denoted as TGNP-DNA), was immobilized onto gold nanoparticles to produce the test-line GNPs (TGNP), which were printed onto the conjugate pad. A second biotinylated cDNA, including a poly-T spacer and a sequence complementary to the 3′ end of the liberated cleavage product (denoted as TL-DNA), was bound to streptavidin and printed onto the nitrocellulose membrane to produce a test line. Finally, control-DNA immobilized onto gold nanoparticles (CGNP-DNA) was mixed with equal amount of TGNP and printed onto the LFD conjugate pad, while control-line cDNA (CL-DNA, FIG. 3B) was printed onto the LFD membrane to generate a control line via hybridization of these two species.

In the presence of EPX, the FS within the DNAzyme is cleaved, releasing the bridging strand into solution, as shown in FIG. 3C, while uncleaved DNAzymes remain bound to the agarose beads in solution and thus cannot migrate along the LFD. Following the cleavage reaction, a quenching buffer containing SDS was added to stop the DNAzyme reaction and impart an anionic charge to proteins in the sample, after which the clear supernatant was pipetted onto the sample pad of the LFD to allow the cleaved bridging DNA to travel towards the adsorbent pad of the strip and bridge between the TGNP and the TL-DNA to form a positive test line along with a positive control line (FIG. 3D, part (i)). If only the control line develops a colour, for example red, the DNAzyme has not cleaved, and the test is negative for EPX (FIG. 3D, part (ii)). The absence of a control line is considered an invalid result.

### Example 5: Evaluation of LFD in Buffer and with Clinical Sputum Samples

### Materials and Methods

### LFD Test in Buffer.

LFD specificity. 100 μL of the BNA-FS-EPDz20M4B-agarose bead complex (from the 500 μL stock made in the above step) was placed in a 1.5 mL microcentrifuge tube and 1 μL of each protein (5 μM in 1×SB) was added to achieve a protein concentration of 50 nM, with buffer was added in the negative control tube. The cleavage reaction was done at RT for 30 min for each of EPX, MPO, TPO, LPO, EDN, ECP, PRGII and RNAse H and a mixture of the latter 7 proteins, with occasional shaking to prevent the beads from settling. 100 μL of the quenching buffer (25 mM HEPES, pH 7.5, 75 mM NaCl, 0.25% Tween20, 0.5% SDS, 1% BSA) was added to each tube. After settling the beads for 5 min, 90 μL of the clear supernatant of each tube was applied to the LFD with a pipette and ran for 15 min before scanning the LFDs. The remaining contents of the reaction tube were imaged with a Chemidoc™ and the fluorescence intensity of each tube (resulting from the cleavage of the DNAzyme) was determined using ImageJ.

LFD images obtained using an EPSON scanner (model: J371A) with a resolution of 1200 dpi and 24-bit color settings. The images were saved as JPEG files and opened in ImageJ software. The image was inverted to convert the red color into blue. The test line was selected in rectangular box and the color intensity (CI) was measured. Note that the area of each rectangle was kept same for all images. The color (signal) intensity just above the test line was measured as background with the same rectangle shape. All CI data was exported to Excel and the background CI was subtracted from the CI of the test line to produce a final CI value. A similar processing method was used for all LFD experiments, including those with buffer and with clinical sputum samples.

LFD Sensitivity. To evaluate sensitivity, 100 μL of the BNA-FS-EPDz20M4B-bead suspension was mixed with 1 μL of EPX stocks of different concentrations to achieve a final concentration range from 0-50 nM EPX in 1×SB. Cleavage reactions were carried out at RT for 30 min as above, followed by addition of an equal volume of quenching buffer. After settling the beads, 90 μL of supernatant from each tube was applied in the LFD and allowed to run for 15 min, followed by scanning and determination of CI values. Fluorescence images of the beads in the tubes was also obtained using a Chemidoc™ imager and fluorescence intensity was quantified using ImageJ.

Nuclease resistance test. Additional nuclease resistance experiments were performed using 250 nM of FS ligated DNAzyme (FS-EPDz20M4) in the presence of SB alone, DNase I, RNase A, RNase2, RNase3 and PRG2, each at 50 nM final concentration. Reaction mixtures were incubated at RT 30 min and quenched by adding 2× gel loading dye. All the reaction mixtures were analyzed by 10% dPAGE and imaged by using Chemidoc™ fluorescence imager.

LFD Evaluation with Clinical Sputum Samples

Sample Collection and Processing. All experiments with patient samples herein were performed as per the protocols approved by the Hamilton integrated Research Ethics Board (HiREB), St. Joseph's Hospital, Hamilton, project number: 12-3687. Healthy donors were recruited under the approved HiREB protocol #13203 and signed written consent was obtained for this clinical validation study. A total of 38 sputum samples from patients (n=31) or healthy donors (n=7) were used for this disclosure (see Table 3 for details on each patient sample). Patient samples were derived from sputa that were clinically indicated for a cell differential with excess available sample after routine processing was done. Healthy donors were identified as those with no known respiratory disease, infection, or symptoms, not within 8 weeks of any vaccination, non-smoking and generally deemed to be in good health.

Sputum samples were first placed in a petri dish on a black background to visualise the opaque sputum plugs. If the sputa expectorate was too thick, forceps was used to select the sputum plugs, which were then transferred to a pre-weighed fresh conical 15 mL tube. The amount of sputum plug (weight) was calculated by deducting the tube weight. The sputum plugs were split into two equal volumes and the first aliquot was processed using a 4:1 mixture of PBS and 0.1% dithiothreitol (DTT) as per the “gold-standard” clinical method while the second aliquot was dispersed with 8:1 (w:w) HEPES buffer containing 2 mM DTT for testing with the LFD (see below). For the gold-standard clinical method, the sputum was first centrifuged and then the suspended cells were smeared onto a slide to produce cytospin slides. The cells were then stained with Wright's stain, and cellular differentials (eosinophils/neutrophils) were determined by manual counting of eosinophil and neutrophil cells, and reported as a percentage of a total of 400 cells counted by a cytologist (validated for clinical routine use, see Table 3 for cell counts in each of the 27 samples). Matched cell-free supernatants (fluid fraction) were assessed for EPX reactivity by a traditional enzyme-linked immunosorbent assay (ELISA) method, as described in references #9 and #10. Samples were designated as eosinophilic based on the presence of intact eosinophils (≥3%) and/or free eosinophil granules. In the event where the eosinophil numbers may have been masked by high total cell count and neutrophils (in a patient undergoing an infective exacerbation), free eosinophil granules or EPX (ELISA) was used to assess/confirm the underlying eosinophilia. Based on these assays, the samples were stratified as: 1) healthy donor samples, confirmed to have no evidence of inflammation, were indicated as eosinophil (EOS) negative; 2) patient samples with <3% eosinophil content and low neutrophil counts (<64%, sputum total cell count; <9.7×10⁶ cells/gm), denoted as EOS negative; 3) patients with mixed granulocytic sputa with total cell count >9.7×10⁶ cells/gm, neutrophils >64% and eosinophils ≥1.5%, or presence of free eosinophil granules, were indicated as EOS positive; and 4) EOS positive samples with ≥3% eosinophil levels, and/or free eosinophil granules and low neutrophil counts (<64%, sputum total cell count <9.7×10⁶ cells/gm). Mixed granulocytic sputa were considered to be EOS positive as they have evidence of eosinophil activity (free granules or EPX assay) since the eosinophil numbers are masked by high neutrophils on a sputum differential count. Therefore, 28 samples were identified as EOS negatives and 18 were EOS positives which included patients with mixed sputa.

Sputum Processing and Cleavage Test. Processing of samples for LFD analysis utilized a simplified procedure designed to make the overall workflow shorter and more compatible with point-of-care testing. In this case the sputum plugs obtained were dispersed by adding 8× (weight) of HEPES buffer (HB; 50 mM HEPES, 300 mM NaCl, 15 mM MgCl₂, 0.01% Tween20 pH 7.5 including 2 mM DTT) and the samples were dispersed by inverting the tube by hand for 5 min and then settling the dispersal on ice for 2 min without a centrifugation step. The supernatants from the settled dispersed samples were then aliquoted into 250 μL in fresh Eppendorf tubes that included a protease inhibitor cocktail (Roche catalogue number 11697498001, containing a mixture of serine, cysteine, and metalloproteases—1 tablet is dissolved in 50 mL deionized water as per manufacturer's protocol and 10 μL is added to 500 μL of dispersed supernatants) and stored at −20° C. until use.

Prior to evaluating the LFD with patient samples, cleavage reactions were performed with four separate negative patient samples both before and after spiking with EPX to a final concentration of 50 nM and cleavage was evaluated using 10% dPAGE. 6 μL of BNA-FS-EPDz20M4 (150 nM in 1×SB) was mixed with 4 μL of a processed sputum sample and the reaction was run for 30 min at RT, followed by adding 10 μL of 2×GLB and performing 10% dPAGE analysis. Real-time fluorescence was also measured for a both a negative and positive patient sample along with a buffer blank to assess the minimum time needed to generate a significant change in signal for the positive relative to the negative sample and buffer blank.

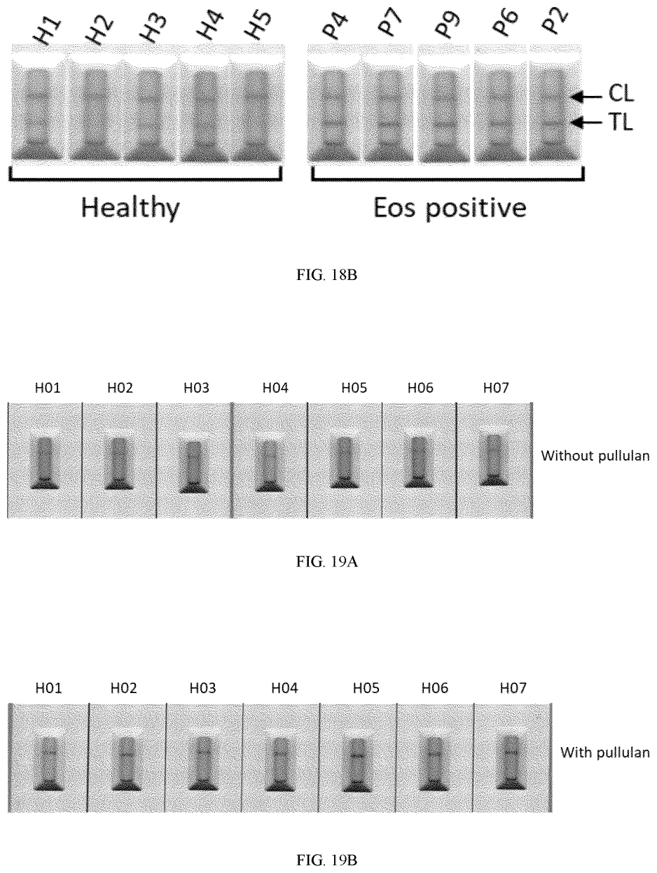

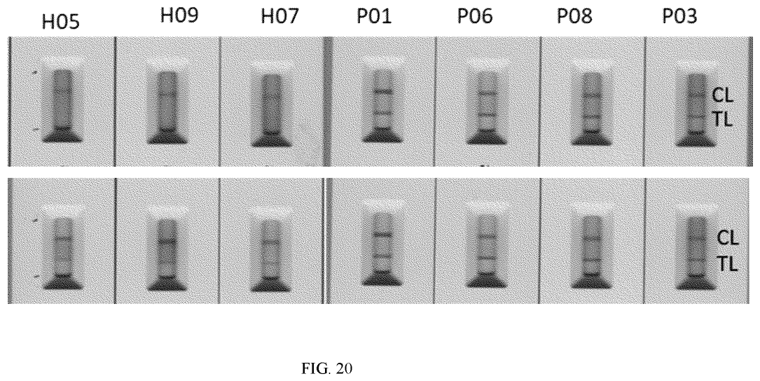

Improvement of LFD testing of sputum samples. LFDs were tested using patient sputum samples under three separate conditions. For method (1), 50 μL of the BNA-FS-EPDz20M4B-bead suspension was mixed with 20 μL of a sputum sample and the cleavage reaction was conducted for 30 min at RT. The reaction was quenched by adding 100 μL of quenching buffer (same as above). After settling the beads, 90 μL of the supernatant was applied in the LFD and allowed to run for 15 min before scanning. Given the significant background obtained using this procedure (see FIG. 18), the LFD assay was modified using a second set of conditions. In method (2), 50 pmole of BNA-FS-EPDz20M4 was immobilized on 100 μL streptavidin agarose beads as described above and suspended in 1 mL 1×SB. 50 μL of this bead slurry was used for each cleavage reaction. 10 μL of sputum sample was added to this 50 μL bead slurry and allowed to react for 15 min at RT and the reaction was then quenched by adding 240 μL of quenching buffer. 90 μL of the supernatant was then applied to the LFD, followed by scanning after 15 min. While the background was suppressed with this method, the control lines were faint and had an inconsistent intensity (FIG. 19A). Method (3) involved adding 0.5% (w/v) pullulan to the quenching buffer while keeping all the conditions same as for Method (2). This method produced strong and consistent control lines with no background (FIG. 19B). Evaluation of test line intensity was also done after sitting the LFDs at RT overnight to determine if test line intensity would slowly increase with time (FIG. 20).

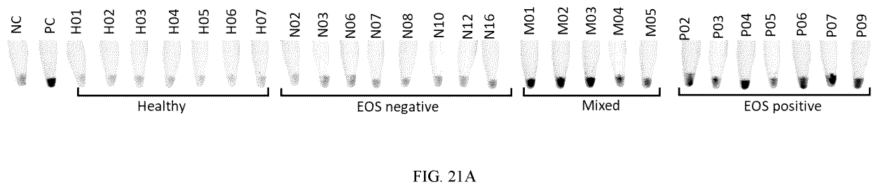

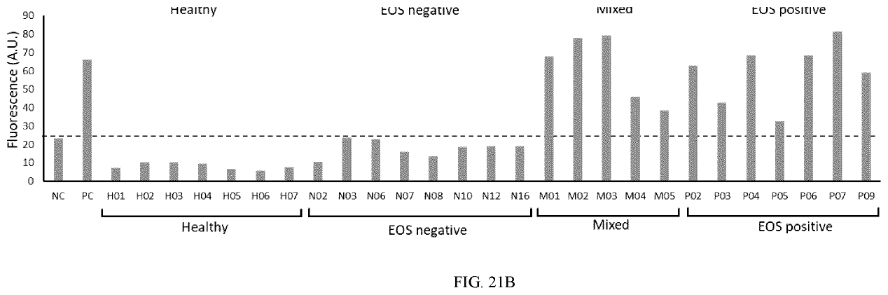

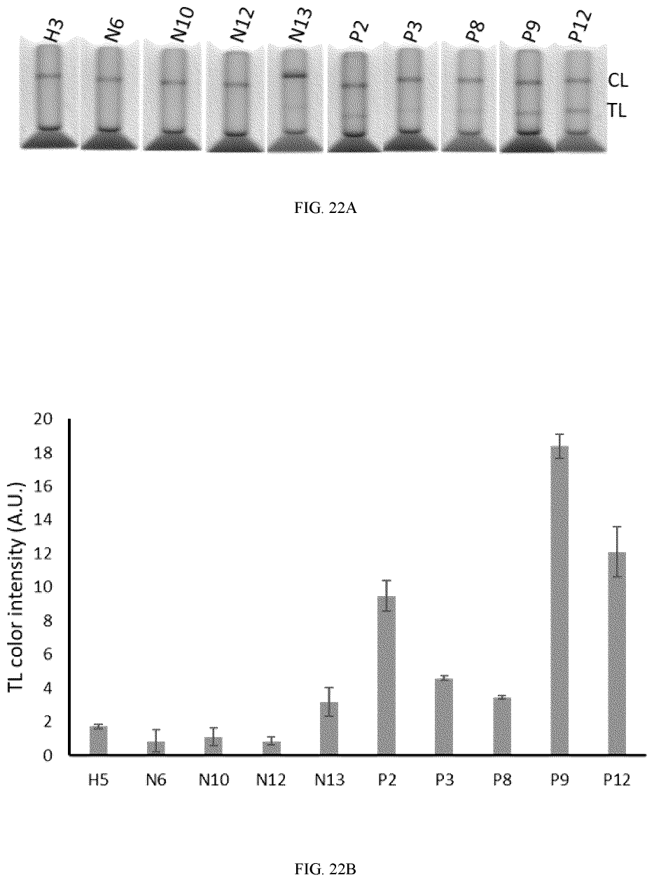

Clinical Validation Study. LFD testing was performed for each of the 38 sputum samples along with a negative control (buffer) and a positive control (50 nM EPX in buffer). Samples included 7 healthy, 16 EOS negative, 6 mixed and 9 EOS positive samples. Each sample was processed as noted above and LFD analysis was performed using Method (3). The LFD test lines were scanned and processed as described above and the background corrected CI values (see above) were plotted. A cut-off value of 2 CI units was assigned as the maximum value that could provide a clinical sensitivity of 100% (no false negatives). A selection of 10 samples (5 negative and 5 positive) with sufficient volume remaining were evaluated in triplicate to determine the reproducibility of the LFD assay (FIG. 22A and FIG. 22B). For a subset of 27 samples, fluorescence imaging was also performed on the tubes after the quenching reaction (see FIG. 21A and FIG. 21B) and were quantified using ImageJ, with a cut-off value of 25 being used as the maximum value that provided a clinical sensitivity of 100%.

### Statistical Analysis of Clinical Validation and Correlation Data.

Test line intensity for negative and positive samples was evaluated using the Kruskal Wallis test with multiple comparison, using a cut-off value of 3.3, to establish P-values between healthy, negative, mixed, and positive samples. Test line colour intensity was also compared with the percent eosinophils and absolute eosinophils detected in the sputum using the gold-standard clinical method, as well as with EPX concentration assayed by an established ELISA method and OD values obtained from an aptamer-based EPX pulldown assay. Data were fit with linear least squares analysis to determine correlation co-efficients and P-values in each case.

### Results and Discussion

LFD test in buffer. The cleavage performance of BNA-FS-EPDz20M4 was first tested in buffer using EPX as along with LPO, TPO, MPO and buffer as negative controls. The dPAGE data (FIG. 3E) show that the extended FS sequence did not affect the cleavage activity of the DNAzyme in the presence of EPX. The LFD for EPX detection in buffer was then evaluated using the immobilized RCD and a cleavage time of 30 min. The reaction was quenched by adding an equal volume of quenching buffer (50 mM HEPES, pH 7.5, 150 mM NaCl, 15 mM MgCl₂, 1% BSA, 0.25% Tween20, 0.25% SDS). After settling the beads for 2 min, 90 μL of the supernatant was applied to the LFD, eluted for 15 min and scanned with an HP scanner. FIG. 3F shows that a low background TL signal was obtained for all control proteins (at 50 nM), while a strong test line appeared with 50 nM EPX present (see inset images) that was 6-fold more intense than for any control protein, indicating that the DNAzyme-based LFD retained selectivity for EPX. Reducing the cleavage reaction time to 15 min and increasing the volume of quenching buffer to 4× the volume of the cleavage reaction could effectively eliminate these background signals, while also reducing overall assay time, as discussed in more detail below.

The fluorescence signal of the DNAzyme remaining on the beads was also measured to assess the cleavage reaction, given that the fluorescein dye remains attached to the sequence fragment bound on the beads after cleavage, but will show more intense fluorescence owing to dequenching upon cleavage. The fluorescence images (see inset to FIG. 3F) clearly indicate a stronger signal is obtained in the presence of EPX, again highlighting the potential for utilizing a fluorescence readout to complement the LFD results.

The sensitivity of the LFD was also evaluated using varying concentration of EPX in buffer. Once again, a 30 min cleavage reaction was performed followed by quenching and a 15 min LFD assay. The results (FIG. 3G) revealed that the LFD produced a test line that could be detected by eye with as little as 6 nM of EPX. In addition, the fluorescence intensity of the reaction tubes following the cleavage reaction clearly increased with EPX concentration, showing that the immobilized DNAzyme retained EPX-dependent fluorescence signalling abilities. Given that a typical eosinophil cells (EOS) cut-off for indication of disease is 400,000 EOS/mL in blood or sputum using a 3% cut-off and EPX is present at 12 μg per 106 EOS cells, the clinical cut-off is ˜4.8 μg/mL, which corresponds to 70 nM EPX, with the clinical range of EPX concentrations spanning 1 nM to 1 μM. Sputum samples are diluted 40× prior to the DNAzyme reaction (see below) and hence ˜2 nM of EPX is present at the cut-off value of 3%, which is in line with limit of detection (LOD) of 3 nM in the instant disclosure.

Evaluation of LFD with clinical sputum samples. To validate the RCD-based LFD, a total of 38 sputum samples from patients (n=31) or healthy donors (n=7) was obtained, using either saline-induction (n=15) or spontaneous expectorant (n=23) (both samples give sputum cell counts that are valid, repeatable, and comparable with their informed consent and with all experiments performed as per the protocols approved by the Hamilton Integrated Research Ethics Board (HiREB), St. Joseph's Hospital, Hamilton, under project numbers 12-3716 (patient samples) or 13203 (healthy donors). Healthy donors were identified as non-smokers with no known respiratory disease, infection or symptoms, who were beyond 8 weeks of any vaccination and were generally deemed to be in good health.

The LFD was evaluated using minimally processed sputum that was simply diluted 8-fold in selection buffer containing 2 mM DTT to break mucin and disulphide bonds followed by analysis of the cell-free fluid fraction for fluid-phase analytes released by the airway cells. Separation of cell debris was done by simple gravity sedimentation (2 min) to remove cellular and heavy mucous debris that remains undispersed, as well as heavier complexes of bound immunoglobulins. The cell-free supernatant contains fluid-phase analytes ranging from microgram levels of free immunoglobulins, released granule proteins such as ECP, EDN and EPX, fibrinogens, free mucin moieties, cytokines (IL-5, IL-8 etc.), and nucleic acids including microRNAs and DNA from extracellular traps, which are specific to certain disease states and may not be constitutively present in all sputa.

To allow comparison of the LFD results to “gold-standard” sputum cytometry results, the sputum plugs were collected and processed to produce two sputum samples, one that was centrifuged to remove cellular debris for the sputum cytometry method and a second aliquot for use with the LFD, where the sputum plug was processed as described above.

Cellular differentials (eosinophils/neutrophils) were determined by manual counting of eosinophil and neutrophil cells, and reported as a percentage of a total of 400 cells (see Table 3 for cell counts obtained for each of the 38 samples). Matched cell-free supernatants (fluid fraction) were assessed for EPX concentration by a traditional ELISA method. Samples were designated as eosinophilic based on the presence of intact eosinophils (≥3%) and/or free eosinophil granules or increased EPX concentrations (by conventional ELISA). Based on these assays, the samples were stratified as: 1) healthy donor samples, indicated as eosinophil (EOS) negative; 2) EOS negative patient samples with <3% eosinophil content and low neutrophil counts (<64%); 3) mixed granulocytic sputa with neutrophils >64% and eosinophils ≥1.5%, or presence of free eosinophil granules or EPX ELISA values above 50 ng/mL, which are considered as EOS positive; and 4) EOS positive samples with ≥3% eosinophil levels, and/or free eosinophil granules and low neutrophil counts (<64%). A total of 23 samples were identified as EOS negatives and 15 as EOS positives, which included patients with mixed granulocytic sputa (Table 3). The samples were either used immediately or aliquoted and stored at −20° C. in an anti-protease cocktail for later analysis to prevent degradation of the EPX target protein.

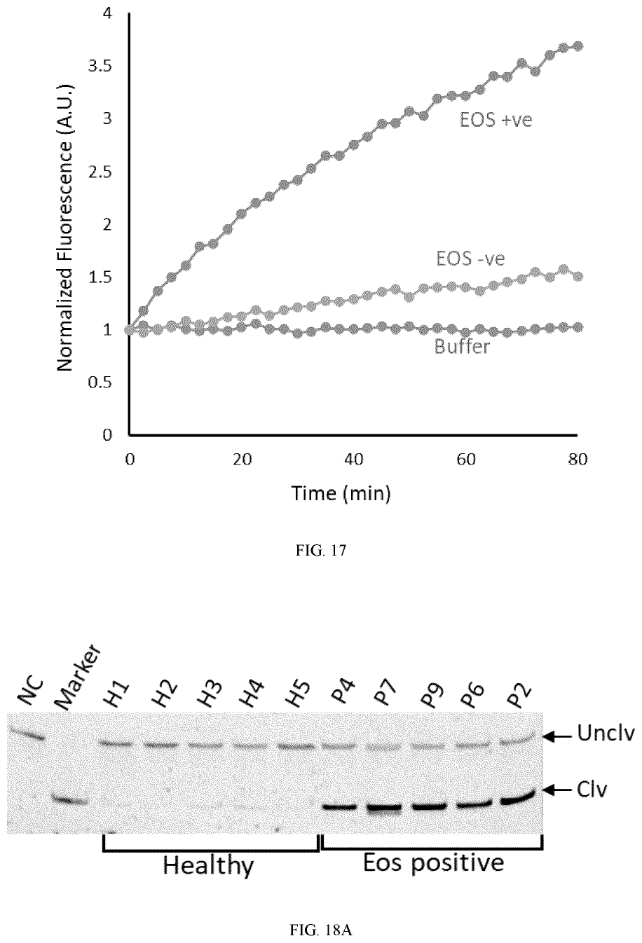

To assess the potential for false positives owing to sputum-induced cleavage of the RCD, dPAGE was used to test the cleavage of EPDz20M4 with a series of negative patient sputum samples before and after spiking with 50 nM EPX. As shown in FIG. 16, the negative sputum samples without added EPX showed little or no cleavage, with only one sample (19408) producing a statistically significant cleavage band (corresponding to <1% cleavage) after 30 min. However, all spiked samples produced strong cleavage bands after 30 min (12-14% cleavage), demonstrating that the RCD retained high selectivity even in the complex sputum sample. Real-time fluorescence assays (FIG. 17) showed the low rate of background cleavage when using EOS negative sputum sample, which only rose above the baseline signal after ˜15 min and was much lower than the signal obtained from an EOS positive sample, showing that positive and negative samples could be discriminated with minimal background cleavage.

LFD performance was improved by comparing three sample processing methods. Initially (Method (1)), the sputum was first processed as described above, diluted 2.5-fold with SB, and a 30 min cleavage time was used followed by a 4× dilution with quenching buffer containing 0.25% sodium dodecyl sulfate (SDS) and a 15 min LFD elution time. In this case, both EOS positive and negative samples produced detectable cleavage bands (FIG. 18A) and LFD test line signals (FIG. 18B), in agreement with the background levels observed from non-target proteins. A modified method (Method (2)) utilized a shorter cleavage reaction (15 min) and a more dilute sputum sample (5× dilution) to reduce background and included a quenching buffer with a higher concentration of SDS (0.5%) to bind to proteins and render them anionic, reducing the potential for electrostatic interactions with TGNP or TL-DNA. These conditions reduced background signals, but also produced inconsistent control lines, as shown in FIG. 19A. To overcome this issue, a final modification was introduced (Method (3)) that involved the addition of 0.5% (w/v) pullulan in the quenching buffer to increase molecular crowding and sample viscosity, which should increase the extent of hybridization to the TGNP and TL. This final modification resulted in LFDs that produced strong tests lines for EOS positive samples, no background for negative samples, and strong and consistent control lines (FIG. 19B).

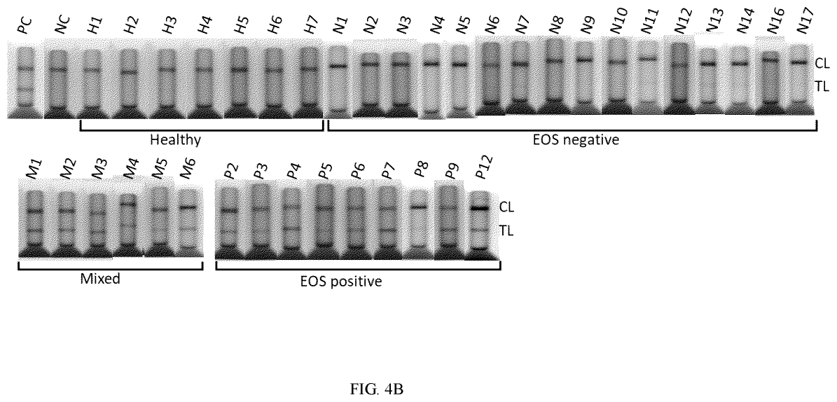

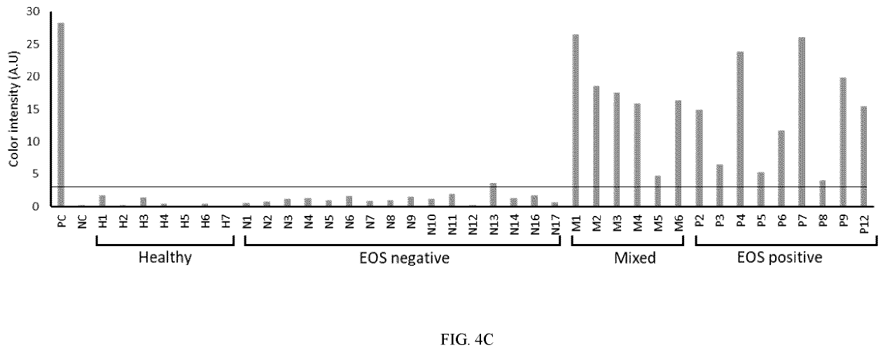

Following the improvement, the 38 sputum samples (7 healthy, 16 EOS negative, 6 mixed and 9 EOS positive) were tested using the LFD with Method (3), which is shown schematically in FIG. 4A. The results indicated that all the EOS positive and mixed samples produced an obvious detectable signal that could be visualized by eye (FIG. 4B) while most of the healthy and EOS negative samples did not produce visually detectable signals, making it possible to use the LFD for qualitative (YES/NO) determinations of airway eosinophilia (<3% and/or no free eosinophil granules (FEGs) as EOS negative and ≥3% and/or evidence FEGs as EOS positive) without the need for an associated reader device. It is important to read the signal within 20 min of running the LFDs, as longer run times can produce increased background test line signals (FIG. 20).

Quantification of the LFD test line colour intensity (CI) using a flatbed scanner and ImageJ (FIG. 4C) allowed for the determination of a clinical cut-off value (solid line in the plot) to assess the accuracy of the LFD for determination of positive and negative patient samples. Samples were tested on different days and with different batches of LFDs, resulting in some variation in background levels on the tests. Using a cut-off of 3.3 (mean+3σ of the negative sample intensity values) resulted in clinical specificity of 100% and sensitivity of 96% for the LFD, highlighting the potential of the LFD for qualitative clinical assessment of airway eosinophilia. Two samples (N13 and P8) were close to the cut-off, even so, this work demonstrates that RCD-based LFDs can be used for testing of patient samples, which can show significant variability in consistency and composition, including the presence of high neutrophil levels, EOS degranulation or auto-antibodies.

Fluorescence imaging performed on a selection of the reaction tubes after quenching the cleavage reaction also showed that the fluorescence signals in the tubes were consistent with the LFD results, and that a cut-off value of 25 fluorescence units (3σ of the mean of the negative sample intensity also produced similar clinical specificity and sensitivity values when compared to the LFD (FIG. 21A and FIG. 21B).

To assess the reproducibility of the clinical testing results, a subset of samples, including N13 and P8, were assessed in triplicate. As shown in FIG. 22B, the error bars were relatively small, with errors (based on ±3σ of the mean) ranging from ±0.2 colour intensity (CI) units to ±2 CI units. The data show samples N13 and P8 overlap, within error, indicating that these samples would produce inconclusive results. P8 has 0% eosinophils owing to degranulation (Table 3), and hence should be considered a borderline positive sample even with cytometry.

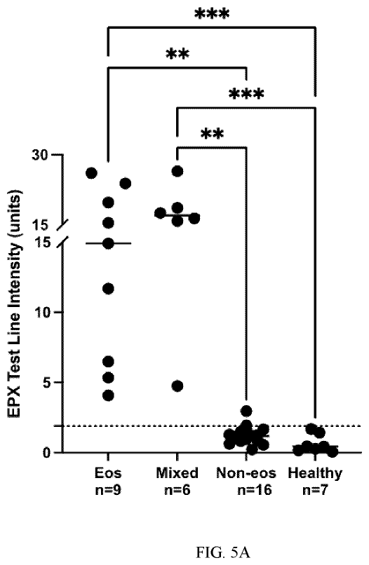

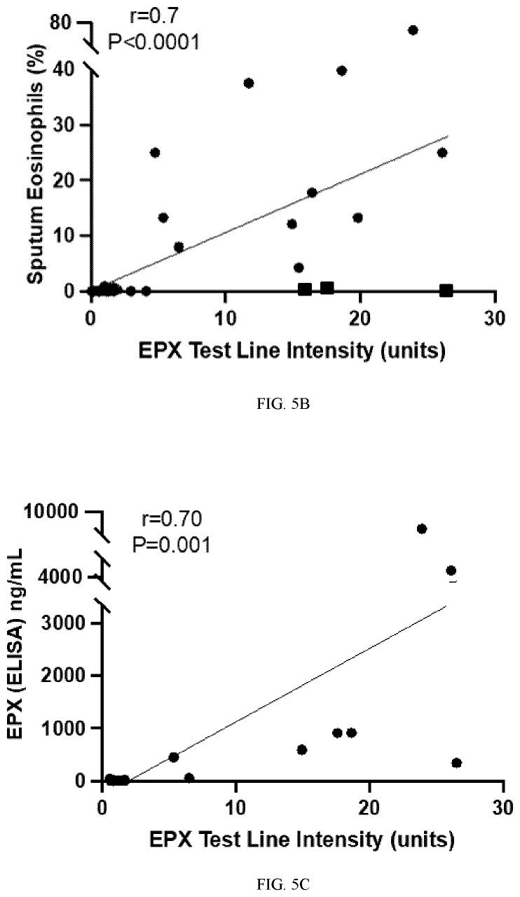

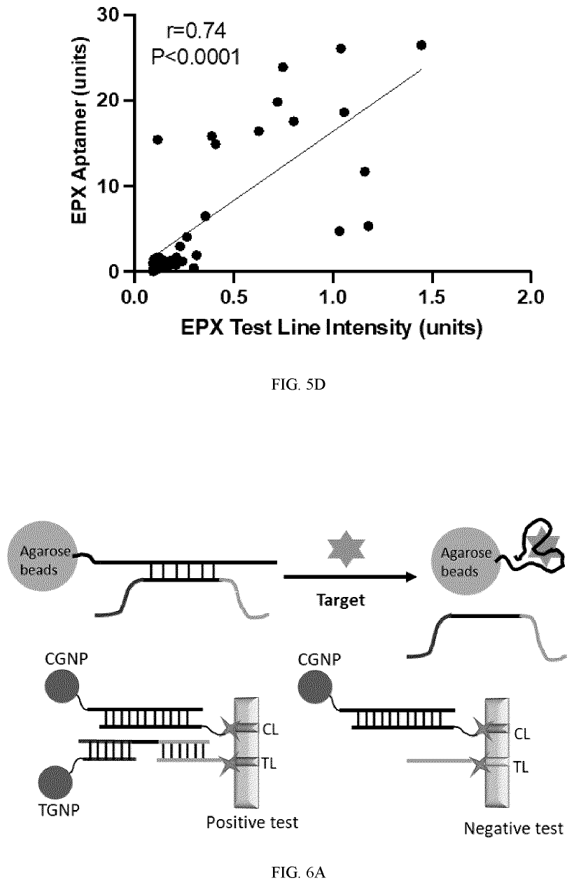

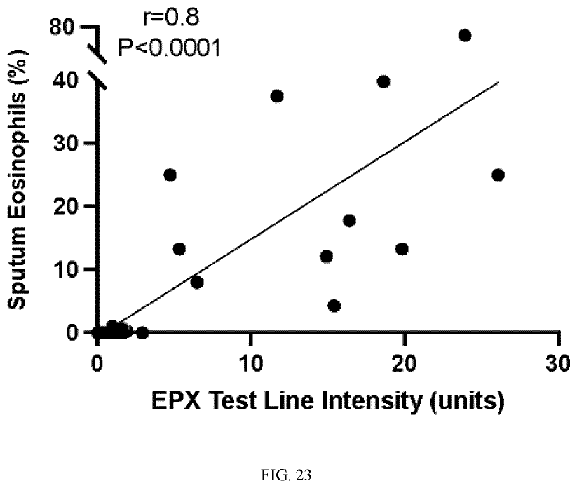

Additional statistical analysis of the LFD data was performed to assess the performance of the LFD assay for determination of positive, mixed, healthy and negative samples (FIG. 5A). The results show that the colour intensity for test lines was significantly greater for EOS positive samples compared to non-eosinophilic samples (P<0.003 in all cases, Kruskal Wallis test with multiple comparison), including mixed granulocytic sputum samples with a sputum eosinophil differential <3% but high EPX levels determined by ELISA, where routine sputum cytometry using percentage of eosinophils would have failed. The test line colour intensity further correlated with the percent eosinophils (FIG. 5B, r=0.70, P=0.003) and absolute eosinophils (r=0.72, P<0.001) detected in the sputum using the cytometry method. However, the correlation was again impacted by the presence of mixed granulocytic sputum samples (total cell count >10 million/gm, neutrophils >65%, eosinophils >1.5 and/or FEGs) where the eosinophil numbers are masked by high levels of neutrophils combined with degranulation of EOS cells, which prevented them from being counted and thus were assigned zero eosinophils by cytometry (M1, M3 and M4 in Table 3, square points in the plot). Removal of the three square points corresponding to these samples resulted in an improvement in the correlation co-efficient to r=0.82 (FIG. 23). The TL intensity also correlated relatively well with EPX concentrations (FIG. 5C, r=0.70, P=0.001) assayed by an established ELISA method, though again the correlation is expected to be impacted by the presence of autoantibodies, which would reduce measured EPX levels for ELISA relative to the LFD, as is evident from the negative bias observed in FIG. 5C. Finally, the LFD test line intensity was compared to the optical density (OD) values obtained using aptamer based EPX assay, providing a correlation value of r=0.74 (FIG. 5D). The relatively poor correlation is likely due to much higher background levels for negative samples in the aptamer assay, along with inherent differences in the dynamic ranges for each assay (0-1000 nM for the aptamer assay, 0-˜50 nM for the DNAzyme assay), and the fact that both assays produce non-linear responses to EPX concentration due to saturation of binding sites on either bead surfaces (aptamer assay) or the LFD test line (current assay). The DNAzyme-based assay also compares favourably to other EPX assays (antibody lateral flow, ELISA, and aptamer-based assay) in terms of assay time, number of steps, LOD and dynamic range, as shown in Table 4.

For comparison, an aptamer-based EPX lateral flow assay was produced. The aptamer-based LFD was designed similarly to the DNAzyme-based LFD, with the aptamer releasing a bridging DNA sequence that is captured on the LFD to bridge a TGNP-DNA and TL-DNA to produce a line (FIG. 6A). The device fabrication was done following the same procedure used to produce the DNAzyme based LFD, using the same sequences for TGNP-DNA, CGNP-DNA, TL-DNA and CL-DNA (see FIG. 6B). The 5′ biotinylated aptamer (denoted as EPX-APT5BT, FIG. 6B) was converted into a structure switching construct through the partial hybridization of a short complementary sequence that contained two overhangs complementary to the TGNP-DNA and TL-DNA, respectively (EPX-BA in FIG. 6B). 100 pmole of the hybridized aptamer-EPX-BA duplex was immobilized onto 100 μL agarose beads slurry using the same method used for the DNAzyme LFD assay. The beads were then washed 5× with SB (SB: 50 mM HEPES, 150 mM NaCl, 15 mM MgCl2, 0.01% Tween 20, pH 7.5). The beads were resuspended in 1 mL SB. For LFD experiments, 100 μL of the aptamer-EPX-BA duplex bead slurry was transferred to a fresh microcentrifuge tube. 20 μL of the 8× diluted sputum sample (N6 negative and P5 positive samples prepared identically to the sample for the DNAzyme LFD), or 8× HB/DTT alone, was added to tube. The mixture was incubated at room temperature with vertical rotation to prevent sedimentation. After 30 of incubation, the beads were settled using a benchtop minicentrifuge. 100 μL of the supernatant was carefully transferred to a new tube. 1 μL of 10% Tween 20 with or without 10% SDS (w/v) was added to the supernatant and the entire volume was applied to the LFD. The photographs were taken at 15 min elution time using an EPSON scanner (model: J371A). The images were processed by ImageJ software and are shown in FIG. 6C (no SDS) and FIG. 6D (0.1% SDS final concentration).

### DISCUSSION

A key advance in the current disclosure is the development of the first allosteric RNA-cleaving DNAzyme that was obtained directly using in vitro selection for a desired protein marker. Many conditions, including heart disease, various cancers and others have highly specific protein biomarkers that can be used to enable rapid diagnostic tests. Hence, it is critical to be able to generate highly sensitive and selective MREs for specific protein markers. This is not currently possible using either the rational design approach, owing to challenges with the design of functional aptazymes, or through the use of cellular media, which produces aptazymes with unknown targets and provides no mechanism to bias the selection toward a specific protein target.

Selection of a highly selective RCD required only minimal counter selection targets. In the work described herein, a total of three proteins were used for counter selection, all of which were peroxidases with high sequence homology to the desired target. The final RCD was not only able to discriminate EPX against the counter selection targets, but also showed remarkable selectivity against protein targets not present during counter selection. Importantly, the RCD was highly selective against two non-specific eosinophil proteins, EDN and ECP, both of which also have ribonuclease activity, and thus might be able to cleave the RCD non-specifically. This finding indicates that generation of protein-selective RCDs can be performed without the need for complex media or extensive counter selection steps, shortening the overall selection time and cost.

A further demonstration of the selectivity of the RCD is its ability to function in sputum. While there are examples of RCDs functioning in other biological media (e.g., faeces or urine), these RCDs were selected using the non-directed cellular media method, and as such had a much greater number of components in the counter selection media. This disclosure shows that even minimal counter selection can lead to clinically useful DNAzymes which are resistant to background cleavage by components in biological samples.

This disclosure also represents only the second time that an assay based on an RCD has been validated using patient samples, and the first use of an RCD for testing patient sputum samples. Given the variability in the consistency and composition of patient sputum, and the potential for interferences related to patient co-morbidities or the presence of high neutrophil levels in patient sputum, it is remarkable that the EPX RCD showed excellent clinical sensitivity and specificity (100% for both), highlighting the potential of this RCD for developing respiratory diagnostics.

The development of a lateral flow device that was compatible with testing for EPX in patient sputum is also significant. Up to now, only a handful of RCD-based LFDs have been reported. While many aptamer-based LFDs have been reported, most have been evaluated only with spiked biological samples, with only a few examples of testing in clinical samples, which were typically highly diluted.⁸ In this disclosure, inventors found that a combination of high concentrations of SDS (to screen protein charges) and pullulan (which aided in hybridization via molecular crowding) was found to result in the complete removal of background in the test line, making it possible to read the LFD visually without the need for an external reader. The entire assay from sample collection to readout on the LFD required only 45 min, which should allow for implementation at the point of care (doctor's office, respiratory clinic, etc.), providing the ability to diagnose the patient and provide follow up decisions during a single visit.

The ability to detect EPX at the point of care is expected to have a major impact on patient care since airway eosinophilia may not always be concurrent with circulatory eosinophil counts. Given the accuracy of the LFD for detection of EPX in clinical sputum samples, the assay should allow rapid identification of airway eosinophilia, enable initiation and monitoring of response to anti-inflammatory treatments such as corticosteroids and anti-eosinophil biologics, and allow evaluation of new therapies directed against eosinophils. A key advantage of the assay is the ability to detect EPX in eosinophilic airway samples even when there are high levels of anti-EPX autoantibodies present, which was not possible using antibody-based lateral flow methods. The ELISA method also produced low OD values even for positive samples with high TL intensity values, suggesting autoantibody interference and possible masking of epitopes. This highlights a key advantage of the DNAzyme as it does not appear to be impacted by sputum autoantibodies. The low cost and portability of LFDs also makes them better suited to use in low-income countries with tropical climates and high humidity, where more costly assays may not be feasible.

In addition to asthma, this assay also has the potential to be adapted to a range of other biological samples such as bronchoalveolar lavage, saliva, nasal secretions, urine, or intestinal secretions including stool samples, to recognize and treat a range of eosinophilic disorders such as chronic rhinosinusitis, eosinophilic esophagitis, eosinophilic granulomatosis with polyangiitis (vasculitis), eosinophilic enteritis, and others. As such, this could provide a starting point for further utilization of the LFD in clinical management of patients, and translational research.

### CONCLUSION

Disclosed herein is the first example of the direct in vitro selection of a protein-binding allosteric RCD, which produced a high affinity DNAzyme for a desired protein biomarker (EPX) with excellent selectivity against allosteric activation by related proteins and resistance to nucleases. The new DNAzyme was shown to be compatible with detection of EPX in patient sputum samples, and was able to provide EPX-dependent fluorescence signal outputs in as little as 10 min. An improved version of the DNAzyme was used to develop a simple lateral flow device and associated assay, providing a method for instrument-free detection of EPX in sputum with a total assay time of 45 min. The LFD was evaluated using 38 patient sputum samples (7 healthy, 16 non-eosinophilic, 15 eosinophilic), producing a clinical sensitivity of 100% and specificity of 96%, demonstrating the potential of the new LFD for rapid diagnosis of airway eosinophilia. This work sets the stage for the development of protein-selective RCDs and for development of simple and rapid clinical diagnostic tests using such recognition elements.

While the present disclosure has been described with reference to examples, it is to be understood that the scope of the claims should not be limited by the embodiments set forth in the examples, but should be given the broadest interpretation consistent with the description as a whole.

All publications, patents and patent applications are herein incorporated by reference in their entirety to the same extent as if each individual publication, patent or patent application was specifically and individually indicated to be incorporated by reference in its entirety. Where a term in the present disclosure is found to be defined differently in a document incorporated herein by reference, the definition provided herein is to serve as the definition for the term.

### Tables

TABLE 1
Synthetic oligonucleotides used in this disclosure.

| SEQ ID | | | |
|---|---|---|---|
| NO. | Name | Sequence (5′-3′) | Description/comment |
| 1 | DL | ATGCCATCCTACCAAC-N50- | DNA library used in |
| | | GAGCTCTGAACTCG | the selection (N50: 50- |
| | | | base random region) |
| 2 | FP | ATGCCATCCTACCAAC | Forward primer |
| 3 | RP1 | CGAGTTCAGAGCTC | Reverse primer used in |
| | | | first PCR |
| 4 and | RP2 | AAAAAAAAAA AAAAAAAAAA- | Reverse primer used iⁿ |
| 68 | | isp9-CGAGTTCAGAGCTC | 2^{nd} PCR |
| | | (SEQ ID NO: 4- | The 5′ of the |
| | | AAAAAAAAAA AAAAAAAAAA) | deoxycytidine in SEQ |
| | | (SEQ ID NO: 68- | ID NO: 68 is attached |
| | | CGAGTTCAGAGCTC) | through the triethylene |
| | | | glycol spacer (isp9) to |
| | | | SEQ ID NO: 4 |
| 5 | RP-Anti | GAGCTCTGAACTCG | Antisense reverse |
| | | | primer |
| 6 | FS | CTATGAACTGACQRFGACCTCAC | Substrate |
| | | TACCAAG | (F: fluorophore-dT, |
| | | | Q: dabcyl-dT, |
| | | | R: riboadenosine) |
| 7 | LT | GTTGGTAGGATGGCATCTTGGTA | Ligation template to |
| | | GTGAGGTC | ligate FS with DL |
| 8 | MiSEQ- | AATGATACGGCGACCACCGAGAT | Forward primer used |
| | FP1 | CTACACTCTTTCCCTACACGACG | in PCR for Illumina |
| | | CTCTTCCGATCTATGCCATCCTA | sequencing |
| | | CCAAC | |
| 9 | MISEC- | CAAGCAGAAGACGGCATACGAGA | Reverse primer used in |
| | RP73 | TTGACAGGTGACTGGAGTTCAGA | PCR for Illumina |
| | | CGTGTGCTCTTCCGATCTCGAGT | sequencing |
| | | TCAGAGCTC | |
| 10 | EPDz20 | ATGCCATCCTACCAACGGTCGAT | Most active full length |
| | | GGATGAGTAATACAGGCCGGATG | DNAzyme |
| | | GGTACAGTCGTAATCTATTCGAG | |
| | | CTCTGAACTCG | |
| 11 | EPDz20M1 | GGTCGATGGATGAGTAATACAGG | Truncated sequence 1 |
| | | CCGGATGGGTACAGTCGTAATCT | (5′ primer was deleted) |
| | | ATTCGAGCTCTGAACTCG | |
| 12 | EPDz20M2 | ATGCCATCCTACCAACGGTCGAT | Truncated sequence 2 |
| | | GGATGAGTAATACAGGCCGGATG | (3′-primer was |
| | | GGTACAGTCGTAATCTATTC | deleted) |
| 13 | EPDz20M3 | GGTCGATGGATGAGTAATACAGG | Truncated sequence 3 |
| | | CCGGATGGGTACAGTCGTAATCT | (both primers were |
| | | ATTC | deleted) |
| 14 | EPDz20M4 | ATGCCATCCTACCAACGGTCGAT | Truncated sequence 4 |
| | | GGATGAGTAATACAGGCCGGATG | (few addition |
| | | GGTACAGTCG | nucleotides from 3′- |
| | | | end of EPDz20M3 was |
| | | | deleted) |
| 15 | EPDz20M5 | ATGCCATCCTACCAACGGTCGAT | Truncated sequence 5 |
| | | GGATGAGTAATACAGGC | (more nucleotides |
| | | | from 3′-end of |
| | | | EPDz20M4 was |
| | | | deleted) |
| 16 | EPDz20M4B | ATGCCATCCTACCAACGGTCGAT | 3′-biotinylated |
| | | GGATGAGTAATACAGGCCGGATG | EPDz20M4 |
| | | GGTACAGTCGTTTTTTTTTTB | |
| 17 | BNA | CTAATGAGTACCTACTGTCTTTT | Bridging sequence for |
| | | TTTTTTACGTGCCTGGATGATC | the test line; was |
| | | | ligated to the 5′-end of |
| | | | FS |
| 18 | LT2 | TAGTCAGTTCATAGGATCATCCA | Ligation template to |
| | | GAA | ligate FS-EPDz20M4B |
| 19 | BNA-FS- | CTAATGAGTACCTACTGTCTTTT | BNA containing |
| | EPDz20M4B | TTTTTTACGTGCCTGGATGATCC | modified DNAzyme |
| | | TATGAACTGACQRFGACCTCACT | used in LFD |
| | | ACCAAGATGCCATCCTACCAACG | (F: fluorophore-dT, |
| | | GTCGATGGATGAGTAATACAGGC | Q: dabcyl-dT, |
| | | CGGATGGGTACAGTCGTTTTTTT | R: riboadenosine; |
| | | TTTB | B: biotin) |
| 20 | TGNP- | AGACAGTAGGTACTCATTAGTTT | DNA to couple with |
| | DNA | TTTTTTTS | AuNP for test line |
| | | | (S: thiol) |
| | | | (i.e. reagent zone test |
| | | | oligonucleotide) |
| 21 | TL-DNA | BTTTTTTTTTTTAGTCAGTTCAT | DNA to print on the |
| | | AGGATCATCCAG | test line (B: biotin) |
| | | | (i.e. sensor zone test |
| | | | oligonucleotide) |
| 22 | CGNP- | GATGACACTGACTACCCATCTTT | DNA to couple with |
| | DNA | TTTTTTTS | control line AuNP |
| | | | (S: thiol) |
| | | | (i.e. reagent zone |
| | | | control |
| | | | oligonucleotide) |
| 23 | CL-DNA | GATGGGTAGTCAGTGTCATCTTT | DNA to print on the |
| | | TTTTTTTB | control line (B: biotin) |
| | | | (i.e. sensor zone |
| | | | control |
| | | | oligonucleotide) |
| 43 | BNA-FS- | CTAATGAGTA CCTACTGTCT | BNA containing |
| | EPDz20M4 | TTTTTTTTTA CGTGCCTGGA | modified DNAzyme |
| | | TGATCCTATG AACTGAC | used in LFD |
| | | QRF GACCTCACTA | (F: fluorophore-dT, |
| | | CCAAGATGCC ATC | Q: dabcyl-dT, |
| | | CTACCAACGG TCGATGGATG | R: riboadenosine; |
| | | AGTAATACAG GCCGGATGGG | B: biotin) |
| | | TACAGTCGTT TTTTTTTTB | |
| 44 | EPX- | BTTTTTTTTTTATGCCATCCTAC | 5′ biotinylated |
| | APT5BT | CAACCAGGGGGACAGTGCAAAGG | aptamer |
| | | GAGCTCTGAACTCG | B: Biotin |
| 45 | EPX-BA | CTAATGAGTA CCTACTGTCT | SEQ ID NO: 44 |
| | | TTGTCCCCCT GGTTTCTGGA | converted into a |
| | | TGATCCTATG AACTGA | structure switching |
| | | | construct |
| 46 | CGNP- | ACCTGGGGG AGTATTGCGG | S: Thiol |
| | DNA | AGGAAGGTTT TTTS | |
| 47 | CL-DNA | BACCTTCCTCC GCAATACTCC | B: Biotin |
| | | CCCAGGTTTT TT | |
| 69 | FS (base | CTATGAACTGACQRFGACCTCAC | Substrate |
| | sequence | TACCAAG | (F: fluorophore-dT, |
| | of | | Q: dabcyl-dT, |
| | SEQ ID | | R: riboadenosine) |
| | NO: 6) | | |
| 70 | EPDz20M4B | ATGCCATCCTACCAACGGTCGAT | |
| | (base | GGATGAGTA | |
| | sequence | ATACAGGCCGGATGGGTACAGTC | |
| | of | GTTTTTTTT | |
| | SEQ ID | TT | |
| | NO: 16) | | |
| 71 | BNA-FS- | CTAATGAGTACCTACTGTCTTTT | BNA containing |
| | EPDz20M4B | TTTTTTACGT | modified DNAzyme |
| | (base | GCCTGGATGATCCTATGAACTGA | used in LFD |
| | sequence | CTRTGACCT | R: riboadenosine |
| | of | CACTACCAAGATGCCATCCTACC | |
| | SEQ ID | AACGGTCGA | |
| | NO: 19) | TGGATGAGTAATACAGGCCGGAT | |
| | | GGGTACAG | |
| | | TCGTTTTTTTTTT | |
| 72 | TGNP- | AGACAGTAGGTACTCATTAGTTT | DNA to couple with |
| | DNA | TTTTTTT | AuNP for test line |
| | (base | | (i.e. reagent zone test |
| | sequence | | oligonucleotide) |
| | of | | |
| | SEQ ID | | |
| | NO: 20) | | |
| 73 | TL-DNA | TTTTTTTTTTTAGTCAGTTCATA | DNA to print on the |
| | (base | GGATCATCCAG | test line |
| | sequence | | (i.e. sensor zone test |
| | of | | oligonucleotide) |
| | SEQ ID | | |
| | NO: 21) | | |
| 74 | CGNP- | GATGACACTGACTACCCATCTTT | DNA to couple with |
| | DNA | TTTTTTT | control line AuNP |
| | (base | | (i.e. reagent zone |
| | sequence | | control |
| | of | | oligonucleotide) |
| | SEQ ID | | |
| | NO: 22) | | |
| 75 | CL-DNA | GATGGGTAGTCAGTGTCATCTTT | DNA to print on the |
| | (base | TTTTTTT | control line |
| | sequence | | (i.e. sensor zone |
| | of | | control |
| | SEQ ID | | oligonucleotide) |
| | NO: 23) | | |
| 76 | BNA-FS- | CTAATGAGTA CCTACTGTCT | R: riboadenosine |
| | EPDz20M4 | TTTTTTTTTA CGTGCCTGGA | |
| | (base | TGATCCTATG AACTGAC | |
| | sequence | TRT GACCTCACTA | |
| | of SEQ ID | CCAAGATGCC ATCCTACCAA | |
| | NO: 43) | CGATGCCATC CTACCAACGG | |
| | | TCGATGGATG AGTAATACAG | |
| | | GCCGGATGGG TACAGTCGTT | |
| | | TTTTTTTT | |
| 77 | EPX- | TTTTTTTTTTATGCCATCCTACC | |
| | APT5BT | AACCAGGGGG | |
| | (base | ACAGTGCAAAGGGAGCTCTGAAC | |
| | sequence | TCG | |
| | of | | |
| | SEQ ID | | |
| | NO: 44) | | |
| 78 | CGNP- | ACCTGGGGG AGTATTGCGG | |
| | DNA | AGGAAGGTTT TTT | |
| | (base | | |
| | sequence | | |
| | of | | |
| | SEQ ID | | |
| | NO: 46) | | |
| 79 | CL-DNA | ACCTTCCTCC GCAATACTCC | |
| | (base | CCCAGGTTTT TT | |
| | sequence | | |
| | of | | |
| | SEQ ID | | |
| | NO: 47) | | |
| 80 | EPDz20 | CTATGAACTGACTRTGACCTCAC | R: riboadenosine |
| | ligated to | TACCAAGATGCCATCCTACCAAC | |
| | the | GGTCGATGGATGAGTAATACAGG | |
| | substrate | CCGGATGGGTACAGTCGTAATCT | |
| | FS | ATTCGAGCTCTGAACTCG | |
| 81 | EPDz20M4 | CTATGAACTGACTRTGACCT | R: riboadenosine |
| | ligated to | CACTACCAAGATGCCATCCTACC | |
| | the | AACGGTCGATGGATGAGTAATAC | |
| | substrate | AGGCCGGATGGGTACAGTCG | |
| | FS | | |

TABLE 2
Synthetic oligonucleotides of the top 20 DNAzyme sequences obtained from
round 15 selection population based on the frequency (or abundance),
including names and read numbers-each of these 20 sequences were
synthesized and tested for cleavage performance.

| SEQ ID | | | Size/ |
|---|---|---|---|
| NO: | Name | Sequence (5′-3′) | Abundance |
| 24 | EPDz01 | ATGCCATCCTACCAACATCTAGGAGTGT | 86904 |
| | | CACGGGTGATATTTGCCTTAAGGGTGGG | |
| | | AGTGTATAGGAGCTCTGAACTCG | |
| 25 | EPDz02 | ATGCCATCCTACCAACGGTCGATGGTTG | 84699 |
| | | AGCAATACAGGCCGGATGGGTACAGTCG | |
| | | TAATCTATTCGAGCTCTGAACTCG | |
| 26 | EPDz03 | ATGCCATCCTACCAACCCGGGGGGTTA | 48182 |
| | | CGTCGATGGGACAAACTTCAGGGTGGGA | |
| | | AGATCGTCTAGAGCTCTGAACTCG | |
| 27 | EPDz04 | ATGCCATCCTACCAACGGTCGATGGTTG | 26937 |
| | | AGTAATACAGGCCGGATGGGTACAGTCG | |
| | | TAATCTATTCGAGCTCTGAACTCG | |
| 28 | EPDz05 | ATGCCATCCTACCAACGTGGTGCAAGTT | 22982 |
| | | GGAATGGGTGGTACCAACTTAAGGGTGG | |
| | | GATGTTCGGCGAGCTCTGAACTCG | |
| 29 | EPDz06 | ATGCCATCCTACCAACGGTCGATGGTTG | 14547 |
| | | AGCAATACAGGCCGGATGGGTACAGTCG | |
| | | TAGTCTATTCGAGCTCTGAACTCG | |
| 30 | EPDz07 | ATGCCATCCTACCAACGATAAGGGGGTT | 12554 |
| | | GGAGGGTTATGAACTGTGGGCGGCTGGG | |
| | | GGATCAATGTGAGCTCTGAACTCG | |
| 31 | EPDz08 | ATGCCATCCTACCAACCATCTAGGAGTG | 11774 |
| | | TCACGGGTGATATTTGCCTTAAGGGTGG | |
| | | GAATGTATAG GAGCTCTGAACTCG | |
| 32 | EPDz09 | ATGCCATCCTACCAACCCGGCAGGGTTA | 10769 |
| | | CGTCGATGGGACAAACTTCAGGGTGGGA | |
| | | AGATCGTCTAGAGCTCTGAACTCG | |
| 33 | EPDz10 | ATGCCATCCTACCAACAGGTTGATGAGT | 10602 |
| | | CGTAGGTTGTGCCGAAGGGACAGACTTC | |
| | | AGGGTGGGAGGAGCTCTGAACTCG | |
| 34 | EPDz11 | ATGCCATCCTACCAACTCCAAGTAGGTG | 8891 |
| | | GTACCAACTTAAGGGTGGGAAGATCTAA | |
| | | TGAAGGGCTCGAGCTCTGAACTCG | |
| 35 | EPDz12 | ATGCCATCCTACCAACACGGTGCAAGTT | 7371 |
| | | GGAATGGGTGGTACCAACTTAAGGGTGG | |
| | | GATGTTCGGCGAGCTCTGAACTCG | |
| 36 | EPDz13 | ATGCCATCCTACCAACCATCTAGGAGTG | 6962 |
| | | TCTCGGGAGATATTTGCCTTAAGGGTGG | |
| | | GAGTGTATAGGAGCTCTGAACTCG | |
| 37 | EPDz14 | ATGCCATCCTACCAACACGGGAGGAAAT | 6333 |
| | | AGGGGACTAACTTCAGGGTGGGAAGTTA | |
| | | AAAGACGGAGGAGCTCTGAACTCG | |
| 38 | EPDz15 | ATGCCATCCTACCAACGGTCGATGGTTG | 6013 |
| | | AGTAATACAGGCCGGATGGGTACAGTCG | |
| | | TAGTCTATTCGAGCTCTGAACTCG | |
| 39 | EPDz16 | ATGCCATCCTACCAACTGGACGGTTACT | 5909 |
| | | TGGAGTGTGCGGAGCAGATGGACTTCAG | |
| | | GGTGGTCGGCGAGCTCTGAACTCG | |
| 40 | EPDz17 | ATGCCATCCTACCAACATGGTGCAAGTT | 5213 |
| | | GGAATGGGTGGTACCAACTTAAGGGTGG | |
| | | GATGTTCGGCGAGCTCTGAACTCG | |
| 41 | EPDz18 | ATGCCATCCTACCAACCGACAGAGAGGG | 4636 |
| | | GTGGCGGGAGGGTCGAAAGGTACAGGGT | |
| | | GGGTGTGGGGGAGCTCTGAACTCG | |
| 42 | EPDz19 | ATGCCATCCTACCAACCATCTAGGAGTG | 3676 |
| | | TGCACGGGTGATATTTGCCTTAAGGTGG | |
| | | GAGTGCATAGGAGCTCTGAACTCG | |
| 10 | EPDz20 | ATGCCATCCTACCAACGGTCGATGGATG | 2679 |
| | | AGTAATACAGGCCGGATGGGTACAGTCG | |
| | | TAATCTATTCGAGCTCTGAACTCG | |
| 48 | EPDz01 | CATCTAGGAGTGTCACGGGTGATATTTG | size = 86904 |
| | (core | CCTTAAGGGTGGGAGTGTATAG | |
| | sequence) | | |
| 49 | EPDz02 | GGTCGATGGTTGAGCAATACAGGCCGGA | size = 84699 |
| | (core | TGGGTACAGTCGTAATCTATTC | |
| | sequence) | | |
| 50 | EPDz03 | CCGGCGGGGTTACGTCGATGGGACAAAC | size = 48182 |
| | (core | TTCAGGGTGGGAAGATCGTCTA | |
| | sequence) | | |
| 51 | EPDz04 | GGTCGATGGTTGAGTAATACAGGCCGGA | size = 26937 |
| | (core | TGGGTACAGTCGTAATCTATTC | |
| | sequence) | | |
| 52 | EPDz05 | GTGGTGCAAGTTGGAATGGGTGGTACCA | size = 22982 |
| | (core | ACTTAAGGGTGGGATGTTCGGC | |
| | sequence) | | |
| 53 | EPDz06 | GGTCGATGGTTGAGCAATACAGGCCGGA | size = 14547 |
| | (core | TGGGTACAGTCGTAGTCTATTC | |
| | sequence) | | |
| 54 | EPDz07 | GATAAGGGGGTTGGAGGGTTATGAACTG | size = 12554 |
| | (core | TGGGCGGCTGGGGGATCAATGT | |
| | sequence) | | |
| 55 | EPDz08 | CATCTAGGAGTGTCACGGGTGATATTTG | size = 11774 |
| | (core | CCTTAAGGGTGGGAATGTATAG | |
| | sequence) | | |
| 56 | EPDz09 | CCGGCAGGGTTACGTCGATGGGACAAAC | size = 10769 |
| | (core | TTCAGGGTGGGAAGATCGTCTA | |
| | sequence) | | |
| 57 | EPDz10 | AGGTTGATGAGTCGTAGGTTGTGCCGAA | size = 10602 |
| | (core | GGGACAGACTTCAGGGTGGGAG | |
| | sequence) | | |
| 58 | EPDz11 | TCCAAGTAGGTGGTACCAACTTAAGGGT | size = 8891 |
| | (core | GGGAAGATCTAATGAAGGGCTC | |
| | sequence) | | |
| 59 | EPDz12 | ACGGTGCAAGTTGGAATGGGTGGTACCA | size = 7371 |
| | (core | ACTTAAGGGGGGATGTTCGGC | |
| | sequence) | | |
| 60 | EPDz13 | CATCTAGGAGTGTCTCGGGAGATATTTG | size = 6962 |
| | (core | CCTTAAGGGTGGGAGTGTATAG | |
| | sequence) | | |
| 61 | EPDz14 | ACGGGAGGAAATAGGGGACTAACTTCAG | size = 6333 |
| | (core | GGTGGGAAGTTAAAAGACGGAG | |
| | sequence) | | |
| 62 | EPDz15 | GGTCGATGGTTGAGTAATACAGGCCGGA | size = 6013 |
| | (core | TGGGTACAGTCGTAGTCTATTC | |
| | sequence) | | |
| 63 | EPDz16 | TGGACGGTTACTTGGAGTGTGCGGAGCA | size = 5909 |
| | (core | GATGGACTTCAGGGTGGTCGGC | |
| | sequence) | | |
| 64 | EPDz17 | ATGGTGCAAGTTGGAATGGGTGGTACCA | size = 5213 |
| | (core | ACTTAAGGGTGGGATGTTCGGC | |
| | sequence) | | |
| 65 | EPDz18 | CGACAGAGAGGGGTGGCGGGAGGGTCGA | size = 4636 |
| | (core | AAGGTACAGGGTGGGTGTGGGG | |
| | sequence) | | |
| 66 | EPDz19 | CATCTAGGAGTGTCACGGGTGATATTTG | size = 3676 |
| | (core | CCTTAAGGGTGGGAGTGCATAG | |
| | sequence) | | |
| 67 | EPDz20 | GGTCGATGGATGAGTAATACAGGCCGGA | size = 2679 |
| | (core | TGGGTACAGTCGTAATCTATTC | |
| | sequence) | | |

SEQ ID NO: 48-67 are the binding domains of the aptamers of the present disclosure. These sequences can be flanked at the 5′ end by ATGCCATCCTACCAAC (SEQ ID NO: 2), and at the 3′ end by GAGCTCTGAACTCG (SEQ ID NO: 5); produced by reverse primer CGAGTTCAGAGCTC (SEQ ID NO: 3)), which lead to DNAzyme sequences corresponding to SEQ ID NOs: 24-42, and 10, respectively.

TABLE 3
Airway inflammatory status for individual patients based on sputum cytology.

| | | | | | | |
|---|---|---|---|---|---|---|
| | Intact | Free | | Inflammatory | Color | Agreement |
| | Sputum | Eosinophil | | status | (Signal) | with gold |
| Patient | Eosinophils | Granules | Inflammatory | (Eos. = 1, | intensity of | standard |
| Number | (%) | (FEGs) | phenotype | Non-eos. = 0) | the test line | (yes = 1) |

Healthy Controls

| | | | | | | |
|---|---|---|---|---|---|---|
| H1 | 0 | 0 | Pauci | 0 | 1.687 | 1 |
| H2 | 0 | 0 | Pauci | 0 | 0.266 | 1 |
| | | 0 | Pauci | 0 | 1.426 | 1 |
| H3 | 0 | | | | | |
| H5 | 0 | 0 | Pauci | 0 | 0.44 | 1 |
| H6 | 0 | 0 | Pauci | 0 | 0.169 | 1 |
| H7 | 0 | 0 | Pauci | 0 | 0.465 | 1 |
| H8 | 0 | 0 | Pauci | 0 | 0.065 | 1 |

Clinically indicated Patients

| | | | | | | |
|---|---|---|---|---|---|---|
| N1 | 0 | 0 | Trivial | 0 | 0.563 | 1 |
| | | | Neutrophilic | | | |
| N2 | 0 | 0 | Pauci | 0 | 0.823 | 1 |
| N3 | 0.7 | 0 | Pauci | 0 | 1.208 | 1 |
| N4 | 0 | 0 | Infective | 0 | 1.294 | 1 |
| | | | Neutrophilic | | | |
| N5 | 0 | 0 | Pauci | 0 | 0.961 | 1 |
| N6 | 0.5 | 0 | Infective | 0 | 1.661 | 1 |
| | | | Neutrophilic | | | |
| N7 | 0.3 | 0 | Pauci | 0 | 0.844 | 1 |
| N8 | 1 | 0 | Pauci | 0 | 1.004 | 1 |
| N9 | 0.5 | 0 | Trivial | 0 | 1.5 | 1 |
| | | | Neutrophilic | | | |
| N10 | 0 | 0 | Pauci | 0 | 1.165 | 1 |
| N11 | 0.3 | 0 | Pauci | 0 | 1.944 | 0 |
| N12 | 0.3 | 0 | Pauci | 0 | 0.226 | 1 |
| N13 | 0 | 0 | Infective | 0 | 2.963 | 0 |
| | | | Neutrophilic | | | |
| N13 | 0 | 0 | Infective | 0 | 2.963 | 0 |
| | | | Neutrophilic | | | |
| N16 | 0.3 | 0 | Infective | 0 | 1.688 | 1 |
| | | | Neutrophilic | | | |
| N17 | 0 | 0 | Infective | 0 | 0.637 | 1 |
| | | | Neutrophilic | | | |
| M1 | 0 | 0 | Infective | 0 | 26.487 | 1 |
| | | | Neutrophilic | | | |
| M2 | 39.8 | 3 | Eosinophilic | 1 | 18.622 | 1 |
| M3 | 0.5 | 0 | Infective | 0 | 17.578 | 1 |
| | | | Neutrophilic | | | |
| M4 | 0.3 | 0 | Infective | 0 | 15.828 | 1 |
| | | | Neutrophilic | | | |
| M5 | 25 | 3 | Eosinophilic | 1 | 4.752 | 1 |
| M6 | 17.8 | 1 | Mixed | 1 | 16.415 | 1 |
| P2 | 12.1 | 3 | Eosinophilic | 1 | 14.917 | 1 |
| P3 | 8 | 3 | Eosinophilic | 1 | 6.5 | 1 |
| P4 | DG* | 3 | Eosinophilic | 1 | 23.9 | 1 |
| P5 | 13.3 | 3 | Mixed | 1 | 5.352 | 1 |
| P6 | 37.5 | 3 | Eosinophilic | 1 | 11.7 | 1 |
| P7 | 25 | 3 | Eosinophilic | 1 | 26.074 | 1 |
| P8 | 0 | 1 | Eosinophilic | 1 | 4.088 | 1 |
| P9 | 3.5 | 0 | Eosinophilic | 1 | 19.822 | 1 |
| P12 | 4.3 | 1 | Eosinophilic | 1 | 15.433 | 1 |

B—all inflammatory phenotypes have been assigned based on routine sputum cytology from Belda et al., AJRCCM 2000 (reference #11).
Pauci—paucigranulocytic (absence of inflammation), Mixed—mixed granulocytic (indicative of infection, total cell count > 9.7 million/gm, neutrophil > 65% with evidence of eosinophils and/or granules); P—clinically-indicated patient sputum, H—healthy control sputum, N—neutrophilic patient sputum, M—mixed patient sputum; FEGs, free eosinophil granules: 0—none, 1—few, 2—moderate, 3—many

TABLE 4
Comparison of different methods regarding
LOD, steps, assay time and dynamic range.

| | | | | Dynamic |
|---|---|---|---|---|
| Assay type | LOD | Steps involved | Assay time | range |
| Antibody | 500 ng/mL | 3 steps including | 45 min | 7-210 nM |
| LFA | (7 nM) | removal of | | |
| | | autoantibodies | | |
| ELISA | 100 pM | 7 steps (total 15 | More than | 100 pM- |
| | | washes) | 7 hours | 100 nM |
| Aptamer | 1 nM | 6 steps (3 washes) | 60 min | 1-100 nM |
| pull-down | | | | |
| DNAzyme- | 3 nM | 2 steps | 45 min | 3 nM- |
| based LFD | | | | 200 nM |
| (Present | | | | |
| Disclosure) | | | | |

### REFERENCES

1) Breaker, R. R. & Joyce, G. F. A DNA enzyme that cleaves RNA. Chem. Biol. 1, 223-229 (1994).
2) Wu, D., Gao, T., Lei, L., Yang, D., Mao, X. & Li, G. Colorimetric detection of proteins based on target-induced activation of aptazyme. Anal. Chim. Acta. 942, 68-73 (2016).
3) Li, J., Wang, S., Jiang, B., Xiang, Y. & Yuan, R. Target-induced structure switching of aptamers facilitates strand displacement for DNAzyme recycling amplification detection of thrombin in human serum. Analyst. 144, 2430-2435 (2019).
4) Yun, W. et al. Proximity ligation assay induced hairpin to DNAzyme structure switching for entropy-driven amplified detection of thrombin. Anal. Chim. Acta. 1064, 104-111 (2019).
5) Cao, Y., Zhang, H. & Le, X. C. Split Locations and Secondary Structures of a DNAzyme Critical to Binding-Assembled Multicomponent Nucleic Acid Enzymes for Protein Detection. Anal. Chem. 93, 15712-15719 (2021).
6) Soukup, G. A. & Breaker, R. R. Allosteric nucleic acid catalysts. Curr. Opin. Struct. Biol. 10, 318-25 (2000).
7) Ali, M. M., Silva, R., White, D., Mohammadi, S., Li, Y., Capretta, A. & Brennan, J. D. A lateral flow test for Staphylococcus aureus in nasal mucus using a new DNAzyme as the recognition element. Angew. Chem. Int. Ed. Engl. 61, e202112346 (2022).
8) Huang, L., Tian, S., Zhao, W., Liu, K., Ma, X. & Guo, J. Aptamer-based lateral flow assay on-site biosensors. Biosens. Bioelectron. 186, 113279 (2021).
9) McGregor, M. C., Krings, J. G., Nair, P., Castro, M. Am. J. Respir. Crit. Care Med. 199, 433-445 (2019).
10) Salter, B., Lacy, P., Mukherjee, M. Front. Pharmacol. 12, 793409 (2022).
11) J. Belda, J., Leigh, R., Parameswaran, K., O'Byrne, P. M., Sears, M. R., Hargreave, F. E., Am. J. Respir. Crit. Care Med. 161, 475-478 (2000).

1. A catalytic nucleic acid for detecting eosinophil peroxidase comprising a nucleic acid sequence selected from the group consisting of SEQ ID NOs: 10, 12, 14, 16, 24, 25, 27-29, 33-40, 42, 43, 48, 49, 51-53, 57-64, 66, 67, 70, 71, 76, 80, and 81, a functional fragment thereof, or a functional variant thereof.

2. The catalytic nucleic acid of claim 1 comprising the nucleic acid sequence selected from the group consisting of SEQ ID NO: 10, 12, 14, 16, 25, 27, 29, 38, 40, 43, 70, 71, 76, 80, and 81.

3. The catalytic nucleic acid of claim 1 or 2 comprising the nucleic acid sequence of SEQ ID NO: 10 or 14.

4. The catalytic nucleic acid of claim 1, wherein the catalytic nucleic acid detects eosinophil peroxidase with a limit of detection (LOD) of about 3 nM, and/or wherein the nucleic acid is for catalyzing cleavage of a substrate nucleic acid.

5. (canceled)

6. A catalytic nucleic acid probe comprising the catalytic nucleic acid of claim 5 and a detectable substrate.

7. The catalytic nucleic acid probe of claim 6, wherein the detectable substrate comprises a ribonucleotide flanked by a fluorophore-modified nucleic acid residue and a quencher-modified nucleic acid residue.

8. A biosensor for detecting eosinophil peroxidase comprising the catalytic nucleic acid of claim 1, functionalized on and/or in a material, optionally the biosensor is a lateral flow device, and/or optionally the biosensor is for use in screening, diagnostics, guiding therapy, health monitoring, and/or pharmaceutical development.

9-11. (canceled)

12. A lateral flow device for detecting the presence or absence of eosinophil peroxidase in a test sample comprising:
a) a sample pad, for applying i) a mixture comprising the test sample, the catalytic nucleic acid of claim 1 immobilized to a solid support, and a running buffer, or ii) for applying a mixture comprising the test sample, a bridging region released from an activated catalytic nucleic acid of claim 1, and a running buffer, to initiate a lateral flow process, wherein the catalytic nucleic acid comprises a bridging region that is configured for release upon contacting the eosinophil peroxidase, and wherein the bridging region comprises a reagent zone test oligonucleotide binding domain configured for binding to a reagent zone test oligonucleotide by complementarity and a sensor zone test oligonucleotide binding domain configured for binding to a sensor zone test oligonucleotide by complementarity,
wherein the running buffer comprises a quenching buffer, optionally the quenching buffer comprises a denaturant, optionally an anionic denaturant, optionally the quenching buffer further comprises pullulan,
b) a reagent zone adjacent to the sample pad, the reagent zone comprising a reagent zone test oligonucleotide coupled to a nanoparticle and a reagent zone control oligonucleotide coupled to a nanoparticle, wherein the reagent zone control oligonucleotide is configured for binding to a sensor zone control oligonucleotide by complementarity,
c) a sensor zone adjacent to the reagent zone, the sensor zone comprising an immobilized sensor zone test oligonucleotide at a test line, and an immobilized sensor zone control oligonucleotide at a control line, and
d) optionally an absorbent pad,
wherein a signal produced at the test line and a signal produced at the control line indicate the presence of eosinophil peroxidase, and
wherein a signal is not produced at the test line and a signal is produced at the control line indicate the absence of eosinophil peroxidase.

13. The lateral flow device of claim 12, wherein the catalytic nucleic acid comprises the nucleic acid sequence of SEQ ID NO: 43, 71, or 73, the reagent zone test oligonucleotide comprises the nucleic acid sequence of SEQ ID NO: 20 or 72, the sensor zone test oligonucleotide comprises the nucleic acid sequence of SEQ ID NO: 21 or 73, the reagent zone control oligonucleotide comprises the nucleic acid sequence of SEQ ID NO: 22 or 74, and the sensor zone test oligonucleotide comprises the nucleic acid sequence of SEQ ID NO: 23 or 75.

14. The lateral flow device of claim 12, wherein the solid support comprises agarose beads, optionally the catalytic nucleic acid is immobilized to the agarose beads by biotin-streptavidin interaction.

15. (canceled)

16. The lateral flow device of claim 12, comprising paper, a polymer support layer and a hydrophobic material, and/or wherein the nanoparticle is a gold nanoparticle.

17. (canceled)

18. The lateral flow device of claim 12, wherein the sensor zone test oligonucleotide and the sensor zone control oligonucleotide are immobilized on a paper, optionally the paper is nitrocellulose paper.

19. (canceled)

20. A method for detecting the presence of eosinophil peroxidase in a test sample from a subject using the catalytic nucleic acid probe of claim 6, the method comprising:
a) applying the test sample to the catalytic nucleic acid probe;
b) detecting a fluorescence signal;
wherein detecting a fluorescence signal above a specified threshold value indicates presence of eosinophil peroxidase in the test sample.

21. The method of claim 20, further comprising diluting the test sample in a buffer before step a), optionally i) the test sample is a sputum sample, and/or ii) the buffer comprises about 2 mM dithiothreitol, and/or iii) the method detects airway eosinophilia in the subject.

22-24. (canceled)

25. A method for detecting the presence or absence of eosinophil peroxidase in a test sample from a subject using the biosensor of claim 8, the method comprising:
a) applying the test sample to the biosensor;
b) detecting a colorimetric signal in a test zone;
wherein detecting a colorimetric signal in a test zone indicates the presence of eosinophil peroxidase in the test sample.

26. The method of claim 25, further comprising diluting the test sample in buffer before step a).

27. The method of claim 25, wherein the test sample is a sputum sample, and/or wherein the buffer comprises about 2 mM dithiothreitol, and/or wherein the method detects airway eosinophilia in the subject.

28-29. (canceled)

30. A method of detecting the presence or absence of eosinophil peroxidase in a test sample from a subject using the catalytic nucleic acid of claim 1, comprising:
applying i) a mixture comprising the test sample, the catalytic nucleic acid immobilized to a solid support, and a running buffer, or ii) a mixture comprising the test sample, a bridging region released from an activated catalytic nucleic acid, and a running buffer, to the sample pad of a lateral flow device,
wherein the catalytic nucleic acid comprises a substrate comprising a ribonucleotide cleavage site,
wherein a quenching buffer is added to the running buffer to quench activity of the catalytic nucleic acid prior to applying the mixture to the sample pad, optionally the quenching buffer comprises a denaturant, optionally an anionic denaturant, optionally the quenching buffer further comprises pullulan,
a) wherein if the test sample comprises eosinophil peroxidase,
i) contacting the eosinophil peroxidase with the immobilized catalytic nucleic acid, and activates the catalytic nucleic acid which cleaves the substrate at the ribonucleotide cleavage site and releases a bridging region comprising the reagent zone test oligonucleotide binding domain and the sensor zone test oligonucleotide binding domain,
ii) allowing the running buffer and the released bridging region to laterally flow into the reagent zone, and a complex is formed between the bridging region and the reagent zone test oligonucleotide,
iii) allowing the running buffer, the complex, and the reagent zone control oligonucleotide to laterally flow to the sensor zone,
iv) allowing the complex to be captured by the sensor zone test oligonucleotide to produce a signal at a test line,
v) detecting the signal in iv) in the sensor zone at the test line, optionally the signal is a color change signal,
vi) allowing the reagent zone control oligonucleotide to be captured by the sensor zone control oligonucleotide to produce a signal at a control line, and
vii) detecting the signal in vi) in the sensor zone at a control line, optionally the signal is a color change signal,
whereby the detection of signals in both v) and vii) is indicative of the presence of eosinophil peroxidase and the correct functioning of the lateral flow device;
or
b) wherein if the test sample does not comprise eosinophil peroxidase, the immobilized catalytic nucleic acid is not activated and no bridging region is released,
i) allowing the running buffer to laterally flow into the reagent zone, and then the running buffer and the reagent zone control oligonucleotide to laterally flow to the sensor zone,
ii) detecting no signal in the sensor zone at the test line,
iii) allowing the reagent zone control oligonucleotide to be captured by the sensor zone control oligonucleotide to produce a signal, and
iv) detecting the signal in the sensor zone at a control line, optionally the signal is a color change signal,
whereby the detection of no signal in ii) and the detection of the signal in iv) are indicative of the absence of eosinophil peroxidase and the correct functioning of the lateral flow device.

31. The method of claim 30, wherein the test sample comprises a clinical sample, optionally the test sample comprises a sputum sample, optionally the method detects airway eosinophilia in the subject.

32-33. (canceled)

34. A kit for detecting eosinophil peroxidase in a test sample, wherein the kit comprises the catalytic nucleic acid probe of claim 6, and instructions for use of the kit.


SEQUENCE LISTING (81 sequences)

SEQ ID NO: 1 - DNA, 80 bp, synthetic construct
  source 1..80: mol_type = other DNA
  misc_feature 17..66: note = n is a, c, g, or t
atgccatcctaccaacnnnnnnnnnnnnnnnnnnnnnnnnnnnnnnnnnnnnnnnnnnnnnnnnnngagctctgaactcg

SEQ ID NO: 2 - DNA, 16 bp, synthetic construct
  source 1..16: mol_type = other DNA
atgccatcctaccaac

SEQ ID NO: 3 - DNA, 14 bp, synthetic construct
  source 1..14: mol_type = other DNA
cgagttcagagctc

SEQ ID NO: 4 - DNA, 20 bp, synthetic construct
  source 1..20: mol_type = other DNA; note = The 3' of the deoxyadenosine is attached through the triethylene glycol spacer to SEQ ID NO: 68
aaaaaaaaaaaaaaaaaaaa

SEQ ID NO: 5 - DNA, 14 bp, synthetic construct
  source 1..14: mol_type = other DNA
gagctctgaactcg

SEQ ID NO: 6 - DNA, 30 bp, synthetic construct
  source 1..30: mol_type = other DNA
  modified_base 13: mod_base = OTHER; note = dabcyl deoxythymidine
  modified_base 14: mod_base = OTHER; note = adenine ribonucleotide
  modified_base 15: mod_base = OTHER; note = fluorescein deoxythymidine
ctatgaactgactatgacctcactaccaag

SEQ ID NO: 7 - DNA, 31 bp, synthetic construct
  source 1..31: mol_type = other DNA
gttggtaggatggcatcttggtagtgaggtc

SEQ ID NO: 8 - DNA, 74 bp, synthetic construct
  source 1..74: mol_type = other DNA
aatgatacggcgaccaccgagatctacactctttccctacacgacgctcttccgatctatgccatcctaccaac

SEQ ID NO: 9 - DNA, 78 bp, synthetic construct
  source 1..78: mol_type = other DNA
caagcagaagacggcatacgagattgacaggtgactggagttcagacgtgtgctcttccgatctcgagttcagagctc

SEQ ID NO: 10 - DNA, 80 bp, synthetic construct
  source 1..80: mol_type = other DNA
atgccatcctaccaacggtcgatggatgagtaatacaggccggatgggtacagtcgtaatctattcgagctctgaactcg

SEQ ID NO: 11 - DNA, 64 bp, synthetic construct
  source 1..64: mol_type = other DNA
ggtcgatggatgagtaatacaggccggatgggtacagtcgtaatctattcgagctctgaactcg

SEQ ID NO: 12 - DNA, 66 bp, synthetic construct
  source 1..66: mol_type = other DNA
atgccatcctaccaacggtcgatggatgagtaatacaggccggatgggtacagtcgtaatctattc

SEQ ID NO: 13 - DNA, 50 bp, synthetic construct
  source 1..50: mol_type = other DNA
ggtcgatggatgagtaatacaggccggatgggtacagtcgtaatctattc

SEQ ID NO: 14 - DNA, 56 bp, synthetic construct
  source 1..56: mol_type = other DNA
atgccatcctaccaacggtcgatggatgagtaatacaggccggatgggtacagtcg

SEQ ID NO: 15 - DNA, 40 bp, synthetic construct
  source 1..40: mol_type = other DNA
atgccatcctaccaacggtcgatggatgagtaatacaggc

SEQ ID NO: 16 - DNA, 66 bp, synthetic construct
  source 1..66: mol_type = other DNA
  modified_base 66: mod_base = OTHER; note = biotinylated thymidine
atgccatcctaccaacggtcgatggatgagtaatacaggccggatgggtacagtcgtttttttttt

SEQ ID NO: 17 - RNA, 45 nt, synthetic construct
  source 1..45: mol_type = other RNA
ctaatgagtacctactgtcttttttttttacgtgcctggatgatc

SEQ ID NO: 18 - DNA, 26 bp, synthetic construct
  source 1..26: mol_type = other DNA
tagtcagttcataggatcatccagaa

SEQ ID NO: 19 - DNA, 141 bp, synthetic construct
  source 1..141: mol_type = other DNA
  modified_base 58: mod_base = OTHER; note = dabcyl deoxythymidine
  modified_base 59: mod_base = OTHER; note = adenine ribonucleotide
  modified_base 60: mod_base = OTHER; note = fluorescein deoxythymidine
  modified_base 141: mod_base = OTHER; note = biotinylated thymidine
ctaatgagtacctactgtcttttttttttacgtgcctggatgatcctatgaactgactatgacctcactaccaagatgccatcctaccaacggtcgatggatgagtaatacaggccggatgggtacagtcgtttttttttt

SEQ ID NO: 20 - DNA, 30 bp, synthetic construct
  source 1..30: mol_type = other DNA
  modified_base 30: mod_base = OTHER; note = thiol-bearing thymidine
agacagtaggtactcattagtttttttttt

SEQ ID NO: 21 - DNA, 34 bp, synthetic construct
  source 1..34: mol_type = other DNA
  modified_base 1: mod_base = OTHER; note = biotinylated thymidine
tttttttttttagtcagttcataggatcatccag

SEQ ID NO: 22 - DNA, 30 bp, synthetic construct
  source 1..30: mol_type = other DNA
  modified_base 30: mod_base = OTHER; note = thiol-bearing thymidine
gatgacactgactacccatctttttttttt

SEQ ID NO: 23 - DNA, 30 bp, synthetic construct
  source 1..30: mol_type = other DNA
  modified_base 30: mod_base = OTHER; note = biotinylated thymidine
gatgggtagtcagtgtcatctttttttttt

SEQ ID NO: 24 - DNA, 79 bp, synthetic construct
  source 1..79: mol_type = other DNA
atgccatcctaccaacatctaggagtgtcacgggtgatatttgccttaagggtgggagtgtataggagctctgaactcg

SEQ ID NO: 25 - DNA, 80 bp, synthetic construct
  source 1..80: mol_type = other DNA
atgccatcctaccaacggtcgatggttgagcaatacaggccggatgggtacagtcgtaatctattcgagctctgaactcg

SEQ ID NO: 26 - DNA, 80 bp, synthetic construct
  source 1..80: mol_type = other DNA
atgccatcctaccaacccggcggggttacgtcgatgggacaaacttcagggtgggaagatcgtctagagctctgaactcg

SEQ ID NO: 27 - DNA, 80 bp, synthetic construct
  source 1..80: mol_type = other DNA
atgccatcctaccaacggtcgatggttgagtaatacaggccggatgggtacagtcgtaatctattcgagctctgaactcg

SEQ ID NO: 28 - DNA, 80 bp, synthetic construct
  source 1..80: mol_type = other DNA
atgccatcctaccaacgtggtgcaagttggaatgggtggtaccaacttaagggtgggatgttcggcgagctctgaactcg

SEQ ID NO: 29 - DNA, 80 bp, synthetic construct
  source 1..80: mol_type = other DNA
atgccatcctaccaacggtcgatggttgagcaatacaggccggatgggtacagtcgtagtctattcgagctctgaactcg

SEQ ID NO: 30 - DNA, 80 bp, synthetic construct
  source 1..80: mol_type = other DNA
atgccatcctaccaacgataagggggttggagggttatgaactgtgggcggctgggggatcaatgtgagctctgaactcg

SEQ ID NO: 31 - DNA, 66 bp, synthetic construct
  source 1..66: mol_type = other DNA
atgccatcctaccaaccatctaggagtgtcacgggtgatatttgccttaagggtgggaatgtatag

SEQ ID NO: 32 - DNA, 80 bp, synthetic construct
  source 1..80: mol_type = other DNA
atgccatcctaccaacccggcagggttacgtcgatgggacaaacttcagggtgggaagatcgtctagagctctgaactcg

SEQ ID NO: 33 - DNA, 80 bp, synthetic construct
  source 1..80: mol_type = other DNA
atgccatcctaccaacaggttgatgagtcgtaggttgtgccgaagggacagacttcagggtgggaggagctctgaactcg

SEQ ID NO: 34 - DNA, 80 bp, synthetic construct
  source 1..80: mol_type = other DNA
atgccatcctaccaactccaagtaggtggtaccaacttaagggtgggaagatctaatgaagggctcgagctctgaactcg

SEQ ID NO: 35 - DNA, 80 bp, synthetic construct
  source 1..80: mol_type = other DNA
atgccatcctaccaacacggtgcaagttggaatgggtggtaccaacttaagggtgggatgttcggcgagctctgaactcg

SEQ ID NO: 36 - DNA, 80 bp, synthetic construct
  source 1..80: mol_type = other DNA
atgccatcctaccaaccatctaggagtgtctcgggagatatttgccttaagggtgggagtgtataggagctctgaactcg

SEQ ID NO: 37 - DNA, 80 bp, synthetic construct
  source 1..80: mol_type = other DNA
atgccatcctaccaacacgggaggaaataggggactaacttcagggtgggaagttaaaagacggaggagctctgaactcg

SEQ ID NO: 38 - DNA, 80 bp, synthetic construct
  source 1..80: mol_type = other DNA
atgccatcctaccaacggtcgatggttgagtaatacaggccggatgggtacagtcgtagtctattcgagctctgaactcg

SEQ ID NO: 39 - DNA, 80 bp, synthetic construct
  source 1..80: mol_type = other DNA
atgccatcctaccaactggacggttacttggagtgtgcggagcagatggacttcagggtggtcggcgagctctgaactcg

SEQ ID NO: 40 - DNA, 80 bp, synthetic construct
  source 1..80: mol_type = other DNA
atgccatcctaccaacatggtgcaagttggaatgggtggtaccaacttaagggtgggatgttcggcgagctctgaactcg

SEQ ID NO: 41 - DNA, 80 bp, synthetic construct
  source 1..80: mol_type = other DNA
atgccatcctaccaaccgacagagaggggtggcgggagggtcgaaaggtacagggtgggtgtgggggagctctgaactcg

SEQ ID NO: 42 - DNA, 80 bp, synthetic construct
  source 1..80: mol_type = other DNA
atgccatcctaccaaccatctaggagtgtgcacgggtgatatttgccttaaggtgggagtgcataggagctctgaactcg

SEQ ID NO: 43 - DNA, 141 bp, synthetic construct; identical to SEQ ID NO: 19
  source 1..141: mol_type = other DNA
  modified_base 58: mod_base = OTHER; note = dabcyl deoxythymidine
  modified_base 59: mod_base = OTHER; note = adenine ribonucleotide
  modified_base 60: mod_base = OTHER; note = fluorescein deoxythymidine
  modified_base 141: mod_base = OTHER; note = biotinylated thymidine

SEQ ID NO: 44 - DNA, 59 bp, synthetic construct
  source 1..59: mol_type = other DNA
  modified_base 1: mod_base = OTHER; note = biotinylated thymidine
ttttttttttatgccatcctaccaaccagggggacagtgcaaagggagctctgaactcg

SEQ ID NO: 45 - DNA, 56 bp, synthetic construct
  source 1..56: mol_type = other DNA
ctaatgagtacctactgtctttgtccccctggtttctggatgatcctatgaactga

SEQ ID NO: 46 - DNA, 32 bp, synthetic construct
  source 1..32: mol_type = other DNA
  modified_base 32: mod_base = OTHER; note = thiol-bearing thymidine
acctgggggagtattgcggaggaaggtttttt

SEQ ID NO: 47 - DNA, 32 bp, synthetic construct
  source 1..32: mol_type = other DNA
  modified_base 32: mod_base = OTHER; note = biotinylated thymidine
accttcctccgcaatactcccccaggtttttt

SEQ ID NO: 48 - DNA, 50 bp, synthetic construct
  source 1..50: mol_type = other DNA
catctaggagtgtcacgggtgatatttgccttaagggtgggagtgtatag

SEQ ID NO: 49 - DNA, 50 bp, synthetic construct
  source 1..50: mol_type = other DNA
ggtcgatggttgagcaatacaggccggatgggtacagtcgtaatctattc

SEQ ID NO: 50 - DNA, 50 bp, synthetic construct
  source 1..50: mol_type = other DNA
ccggcggggttacgtcgatgggacaaacttcagggtgggaagatcgtcta

SEQ ID NO: 51 - DNA, 50 bp, synthetic construct
  source 1..50: mol_type = other DNA
ggtcgatggttgagtaatacaggccggatgggtacagtcgtaatctattc

SEQ ID NO: 52 - DNA, 50 bp, synthetic construct
  source 1..50: mol_type = other DNA
ggtcgatggttgagcaatacaggccggatgggtacagtcgtagtctattc

SEQ ID NO: 53 - DNA, 50 bp, synthetic construct; identical to SEQ ID NO: 52
  source 1..50: mol_type = other DNA

SEQ ID NO: 54 - DNA, 50 bp, synthetic construct
  source 1..50: mol_type = other DNA
gataagggggttggagggttatgaactgtgggcggctgggggatcaatgt

SEQ ID NO: 55 - DNA, 50 bp, synthetic construct
  source 1..50: mol_type = other DNA
catctaggagtgtcacgggtgatatttgccttaagggtgggaatgtatag

SEQ ID NO: 56 - DNA, 50 bp, synthetic construct
  source 1..50: mol_type = other DNA
ccggcagggttacgtcgatgggacaaacttcagggtgggaagatcgtcta

SEQ ID NO: 57 - DNA, 50 bp, synthetic construct
  source 1..50: mol_type = other DNA
aggttgatgagtcgtaggttgtgccgaagggacagacttcagggtgggag

SEQ ID NO: 58 - DNA, 50 bp, synthetic construct
  source 1..50: mol_type = other DNA
tccaagtaggtggtaccaacttaagggtgggaagatctaatgaagggctc

SEQ ID NO: 59 - DNA, 50 bp, synthetic construct
  source 1..50: mol_type = other DNA
acggtgcaagttggaatgggtggtaccaacttaagggtgggatgttcggc

SEQ ID NO: 60 - DNA, 50 bp, synthetic construct
  source 1..50: mol_type = other DNA
catctaggagtgtctcgggagatatttgccttaagggtgggagtgtatag

SEQ ID NO: 61 - DNA, 50 bp, synthetic construct
  source 1..50: mol_type = other DNA
acgggaggaaataggggactaacttcagggtgggaagttaaaagacggag

SEQ ID NO: 62 - DNA, 50 bp, synthetic construct
  source 1..50: mol_type = other DNA
ggtcgatggttgagtaatacaggccggatgggtacagtcgtagtctattc

SEQ ID NO: 63 - DNA, 50 bp, synthetic construct
  source 1..50: mol_type = other DNA
tggacggttacttggagtgtgcggagcagatggacttcagggtggtcggc

SEQ ID NO: 64 - DNA, 50 bp, synthetic construct
  source 1..50: mol_type = other DNA
atggtgcaagttggaatgggtggtaccaacttaagggtgggatgttcggc

SEQ ID NO: 65 - DNA, 50 bp, synthetic construct
  source 1..50: mol_type = other DNA
cgacagagaggggtggcgggagggtcgaaaggtacagggtgggtgtgggg

SEQ ID NO: 66 - DNA, 50 bp, synthetic construct
  source 1..50: mol_type = other DNA
catctaggagtgtcacgggtgatatttgccttaagggtgggagtgcatag

SEQ ID NO: 67 - DNA, 50 bp, synthetic construct; identical to SEQ ID NO: 13
  source 1..50: mol_type = other DNA

SEQ ID NO: 68 - DNA, 14 bp, synthetic construct
  source 1..14: mol_type = other DNA; note = The 5' of the deoxycytidine is attached through the triethylene glycol spacer to SEQ ID NO: 4
cgagttcagagctc

SEQ ID NO: 69 - DNA, 30 bp, synthetic construct; identical to SEQ ID NO: 6
  source 1..30: mol_type = other DNA
  modified_base 14: mod_base = OTHER; note = adenine ribonucleotide

SEQ ID NO: 70 - DNA, 66 bp, synthetic construct; identical to SEQ ID NO: 16
  source 1..66: mol_type = other DNA

SEQ ID NO: 71 - DNA, 141 bp, synthetic construct; identical to SEQ ID NO: 19
  source 1..141: mol_type = other DNA
  modified_base 59: mod_base = OTHER; note = adenine ribonucleotide

SEQ ID NO: 72 - DNA, 30 bp, synthetic construct; identical to SEQ ID NO: 20
  source 1..30: mol_type = other DNA

SEQ ID NO: 73 - DNA, 34 bp, synthetic construct; identical to SEQ ID NO: 21
  source 1..34: mol_type = other DNA

SEQ ID NO: 74 - DNA, 30 bp, synthetic construct; identical to SEQ ID NO: 22
  source 1..30: mol_type = other DNA

SEQ ID NO: 75 - DNA, 30 bp, synthetic construct; identical to SEQ ID NO: 23
  source 1..30: mol_type = other DNA

SEQ ID NO: 76 - DNA, 158 bp, synthetic construct
  source 1..158: mol_type = other DNA
  modified_base 59: mod_base = OTHER; note = adenine ribonucleotide
ctaatgagtacctactgtcttttttttttacgtgcctggatgatcctatgaactgactatgacctcactaccaagatgccatcctaccaacgatgccatcctaccaacggtcgatggatgagtaatacaggccggatgggtacagtcgtttttttttt

SEQ ID NO: 77 - DNA, 59 bp, synthetic construct; identical to SEQ ID NO: 44
  source 1..59: mol_type = other DNA

SEQ ID NO: 78 - DNA, 32 bp, synthetic construct; identical to SEQ ID NO: 46
  source 1..32: mol_type = other DNA

SEQ ID NO: 79 - DNA, 32 bp, synthetic construct; identical to SEQ ID NO: 47
  source 1..32: mol_type = other DNA

SEQ ID NO: 80 - DNA, 110 bp, synthetic construct
  source 1..110: mol_type = other DNA
  modified_base 14: mod_base = OTHER; note = adenine ribonucleotide
ctatgaactgactatgacctcactaccaagatgccatcctaccaacggtcgatggatgagtaatacaggccggatgggtacagtcgtaatctattcgagctctgaactcg

SEQ ID NO: 81 - DNA, 86 bp, synthetic construct
  source 1..86: mol_type = other DNA
  modified_base 14: mod_base = OTHER; note = adenine ribonucleotide
ctatgaactgactrtgacctcactaccaagatgccatcctaccaacggtcgatggatgagtaatacaggccggatgggtacagtcg